(19) United States
(12) Patent Application Publication
(10) Pub. No.: US 2022/0048836 A1
(43) Pub. Date: Feb. 17, 2022

(54) MATERIALS FOR ORGANIC ELECTROLUMINESCENT DEVICES

(71) Applicant: Merck Patent GmbH, Darmstadt (DE)

(72) Inventors: Lara-Isabel RODRIGUEZ, Darmstadt (DE); Aaron LACKNER, Mannheim (DE); Christof PFLUMM, Darmstadt (DE); Amel MEKIC, Darmstadt (DE)

(21) Appl. No.: 17/276,074

(22) PCT Filed: Sep. 9, 2019
(86) PCT No.: PCT/EP2019/073997

§ 371 Date: Mar. 12, 2021

(30) Foreign Application Priority Data
Sep. 12, 2018 (EP) 18194083.4

(51) Int. Cl. C07C 15/20; H01L 51/00

(52) U.S. Cl. CPC C07C 15/20; H01L 51/5016; H01L 51/0055

(57) ABSTRACT

The present invention relates to organic electroluminescent devices comprising a sterically hindered fluorescent perylene emitter compound and a sensitizer compound and to sterically hindered fluorescent perylene emitter compounds.

The present invention relates to organic electroluminescent devices comprising a sterically hindered fluorescent perylene emitter compound and a sensitizer compound selected from compound that exhibit delayed fluorescence and phosphorescent compounds.

The structure of organic electroluminescent devices (OLEDs) in which organic semiconductors are used as functional materials is described, for example, in U.S. Pat. No. 4,539,507. Common emitting materials used in OLEDs are organometallic iridium and platinum complexes which exhibit phosphorescence rather than fluorescence (M. A. Baldo et al., Appl. Phys. Lett. 1999, 75, 4-6). For quantum-mechanical reasons, up to four times the energy efficiency and power efficiency is possible using organometallic compounds as phosphorescent emitters.

In spite of the good results which are achieved with organometallic iridium and platinum complexes as phosphorescent emitters, there is still a need for improvement of OLEDs performances, especially in terms of efficiency, color purity, achieving deep blue colors.

An alternative development to the phosphorescent emitters is the use of emitters which exhibit thermally activated delayed fluorescence (TADF) (e.g. H. Uoyama et al., Nature 2012, vol. 492, 234). These are organic materials in which the energy gap between the lowest triplet state T₁ and the first excited singlet state S₁ is sufficiently small that the S₁ state is thermally accessible from the T₁ state. For quantum-statistical reasons, on electronic excitation in the OLED, 75% of the excited states are in the triplet state and 25% in the singlet state. Since purely organic molecules cannot usually emit efficiently from the triplet state, 75% of the excited states cannot be utilized for emission, which means that it is possible in principle to convert only 25% of the excitation energy to light. If, however, the energy gap between the lowest triplet state and the lowest excited singlet state is sufficiently small, the first excited singlet state of the molecule is accessible from the triplet state by thermal excitation and can be populated thermally. Since this singlet state is an emissive state from which fluorescence is possible, this state can be used to generate light. Thus, in principle, the conversion of up to 100% of the electrical energy to light is possible when purely organic materials are used as emitter. The prior art describes an external quantum efficiency of more than 19%, which is within the same order of magnitude as for phosphorescent OLEDs. It is thus possible with purely organic materials of this kind to achieve very good efficiencies and at the same time to avoid the use of scarce metals such as iridium or platinum.

On the other side, a prerequisite for the presence of a TADF compound is a small gap between the T₁ and S₁ levels, and therefore, the choice of TADF compounds is limited. Furthermore, it is rather difficult to provide TADF compounds having every desired emission color, because the emission spectra are rather broad (usually with a full-width at half maximum, FWHM >80 nm). Additionally, the decay time of the excited states in these compounds is very long (usually >1 μs), which leads to long living excited state with high energy leading to increased degradation in the devices.

Recently, organic electroluminescent devices having, in the emitting layer, a TADF compound as a sensitizer and a fluorescent compound having high steric shielding with respect to its environment as an emitter have been described (for example in WO2015/135624). This device construction makes it possible to provide organic electroluminescent devices which emit in all emission colors, so that it is possible to use the base structures of known fluorescent emitters which nevertheless exhibit the high efficiency of electroluminescent devices with TADF. This is also known as hyperfluorescence.

As an alternative, the prior art describes organic electroluminescent devices comprising, in the emitting layer, a phosphorescent organometallic complex as a sensitizer, which shows mixing of S1 and T1 states due to the large spin-orbit coupling, and a fluorescent compound as an emitter, so that the emission decay time can significantly be shortened. This is also known as hyperphosphorescence.

Hyperfluorescence and hyperphosphorescence are very promising techniques to improve OLEDs properties, especially in terms of deep blue emission. However, further improvements are still necessary with respect to the performance data of OLEDs, in particular with a view to broad commercial use, for example in display devices or as light sources. Of particular importance in this connection are the lifetime, the efficiency and the operating voltage of the OLEDs and as well as the colour values achieved. In particular, in case of blue-emitting OLEDs, there is potential for improvement with respect to the lifetime and the efficiency of the devices.

An important starting point for achieving the said improvements is the choice of the sterically hindered fluorescent emitter compound employed in the electronic device.

In WO 2015/135624, sterically hindered fluorescent emitters based on rubrene are described. However, there is still a need for further sterically hindered fluorescent emitters, especially sterically hindered blue-fluorescent emitters, which lead to OLEDs having very good properties in terms of efficiency and color emission. More particularly, there is a need for deep blue-fluorescent emitters combining very high efficiency, very good life time and suitable color coordinates as well as high color purity.

The present invention is thus based on the technical object of providing electronic devices comprising a sterically hindered blue fluorescent emitter compound in combination with a sensitizer compound. The present invention is also based on the technical object of providing suitable sterically hindered blue fluorescent emitters compounds based on perylene.

It has now been found that the devices, compounds and combination of compounds described below are particularly suitable in the technical field of OLEDs.

A first object of the invention thus relates to an electronic device comprising anode, cathode and at least one organic layer comprising a sterically hindered fluorescent perylene emitter compound, characterised in that the fluorescent perylene emitter compound is represented by the general following formula (I) and in that the organic layer or a layer adjacent to the organic layer on the anode or cathode side comprises a sensitizer compound selected from a compound that exhibits delayed fluorescence or a phosphorescent compound,

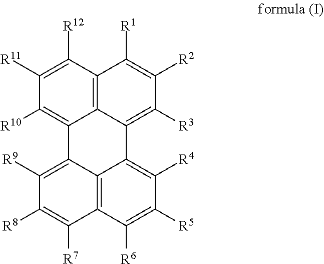

wherein
R¹ to R¹² are each selected, identically or differently, from H, a straight-chain alkyl or alkoxy group having 3 to 20 carbon atoms, a branched or cyclic alkyl or alkoxy group having 3 to 20 carbon atoms, an alkenyl or alkynyl group having 3 to 20 carbon atoms, an aralkyl group, preferably having 7 to 60 carbon atoms, where the above-mentioned groups may each be substituted by one or more radicals R²⁰ and where one or more CH₂ groups in the above-mentioned groups may be replaced by Si(R²⁰)₂, Ge(R²⁰)₂, Sn(R²⁰)₂, C═O, C═S, C═Se, C═NR²⁰, P(═O)(R²⁰), SO, SO₂, NR²⁰, —O—, —S—, —COO— or —CONR²⁰— and where one or more H atoms in the above-mentioned groups may be replaced by D, F, Cl, Br, I, CN or NO₂, or an aromatic ring system having 5 to 60 aromatic ring atoms, which may in each case be substituted by one or more radicals R²⁰;
R²⁰ is on each occurrence, identically or differently, selected from H, D, F, or a straight-chain alkyl group having 1 to 40 carbon atoms, or a branched or cyclic alkyl group having 3 to 40 carbon atoms, or an alkenyl or alkynyl group having 2 to 40 carbon atoms, or an aralkyl group having 7 to 40 carbon atoms, where the above-mentioned groups may each be substituted by one or more radicals R²¹ or an aromatic ring system having 5 to 40 aromatic ring atoms, which may in each case be substituted by one or more radicals R²¹, where two or more radicals R²⁰ may be joined to form an aromatic ring system or a (poly)cyclic alkyl group, which may in each case be substituted by one or more radicals R²¹;
R²¹ is on each occurrence, identically or differently, selected from H, D, F, or a straight-chain alkyl group having 1 to 20 carbon atoms, or a branched or cyclic alkyl group having 3 to 20 carbon atoms, or an alkenyl or alkynyl group having 2 to 20 carbon atoms, or an aromatic ring system having 5 to 30 aromatic ring atoms, where two or more radicals R²¹ may be joined to form an aromatic ring system or a (poly)cyclic alkyl group;
with the proviso that at least two, preferably three, more preferably four, of radicals R¹ to R¹², which are not located at the same benzene ring of the perylene basic skeleton, are other than H.

The following definitions of chemical groups apply for the purposes of the present application:

An aryl group in the sense of this invention contains 6 to 60 aromatic ring atoms, preferably 6 to 40 aromatic ring atoms, more preferably 6 to 20 aromatic ring atoms; a heteroaryl group in the sense of this invention contains 5 to 60 aromatic ring atoms, preferably 5 to 40 aromatic ring atoms, more preferably 5 to 20 aromatic ring atoms, at least one of which is a heteroatom. The heteroatoms are preferably selected from N, O and S. This represents the basic definition. If other preferences are indicated in the description of the present invention, for example with respect to the number of aromatic ring atoms or the heteroatoms present, these apply.

An aryl group or heteroaryl group here is taken to mean either a simple aromatic ring, i.e. benzene, or a simple heteroaromatic ring, for example pyridine, pyrimidine or thiophene, or a condensed (annellated) aromatic or heteroaromatic polycycle, for example naphthalene, phenanthrene, quinoline or carbazole. A condensed (annellated) aromatic or heteroaromatic polycycle in the sense of the present application consists of two or more simple aromatic or heteroaromatic rings condensed with one another.

An aryl or heteroaryl group, which may in each case be substituted by the above-mentioned radicals and which may be linked to the aromatic or hetero-aromatic ring system via any desired positions, is taken to mean, in particular, groups derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, iso-quinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimi-dazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzo-pyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aryloxy group in accordance with the definition of the present invention is taken to mean an aryl group, as defined above, which is bonded via an oxygen atom. An analogous definition applies to heteroaryloxy groups.

An aralkyl group in accordance with the definition of the present invention is taken to mean an alkyl group, where at least one hydrogen atom is replaced by an aryl group.

An aromatic ring system in the sense of this invention contains 6 to 60 C atoms in the ring system, preferably 6 to 40 C atoms, more preferably 6 to 20 C atoms. A heteroaromatic ring system in the sense of this invention contains 5 to 60 aromatic ring atoms, preferably 5 to 40 aromatic ring atoms, more preferably 5 to 20 aromatic ring atoms, at least one of which is a heteroatom. The heteroatoms are preferably selected from N, O and/or S. An aromatic or hetero-aromatic ring system in the sense of this invention is intended to be taken to mean a system which does not necessarily contain only aryl or heteroaryl groups, but instead in which, in addition, a plurality of aryl or heteroaryl groups may be connected by a non-aromatic unit (preferably less than 10% of the atoms other than H), such as, for example, an sp³-hybridised C, Si, N or O atom, an sp²-hybridised C or N atom or an sp-hybridised C atom. Thus, for example, systems such as 9,9′-spirobifluorene, 9,9′-diarylfluorene, triarylamine, diaryl ether, stilbene, etc., are also intended to be taken to be aromatic ring systems in the sense of this invention, as are systems in which two or more aryl groups are connected, for example, by a linear or cyclic alkyl, alkenyl or alkynyl group or by a silyl group. Furthermore, systems in which two or more aryl or heteroaryl groups are linked to one another via single bonds are also taken to be aromatic or heteroaromatic ring systems in the sense of this invention, such as, for example, systems such as biphenyl, terphenyl or diphenyltriazine.

An aromatic or heteroaromatic ring system having 5-60 aromatic ring atoms, which may in each case also be substituted by radicals as defined above and which may be linked to the aromatic or heteroaromatic group via any desired positions, is taken to mean, in particular, groups derived from benzene, naphthalene, anthracene, benzanthracene, phenanthrene, benzophenanthrene, pyrene, chrysene, perylene, fluoranthene, naphthacene, pentacene, benzopyrene, biphenyl, biphenylene, terphenyl, terphenylene, quaterphenyl, fluorene, spirobifluorene, dihydrophenanthrene, dihydropyrene, tetrahydropyrene, cis- or trans-indenofluorene, truxene, isotruxene, spiro-truxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quino-line, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzo-pyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or combinations of these groups.

For the purposes of the present invention, a straight-chain alkyl group having 1 to 40 C atoms or a branched or cyclic alkyl group having 3 to 40 C atoms or an alkenyl or alkynyl group having 2 to 40 C atoms, in which, in addition, individual H atoms or CH₂ groups may be substituted by the groups mentioned above under the definition of the radicals, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclo-pentenyl, hexenyl, cyclohexenyl, heptenyl, cycloheptenyl, octenyl, cyclooctenyl, ethynyl, propynyl, butynyl, pentynyl, hexynyl or octynyl. An alkoxy or thioalkyl group having 1 to 40 C atoms is preferably taken to mean methoxy, trifluoro-methoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethyl-hexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The formulation that two or more radicals may form a ring with one another is, for the purposes of the present application, intended to be taken to mean, inter alia, that the two radicals are linked to one another by a chemical bond. This is illustrated by the following schemes:

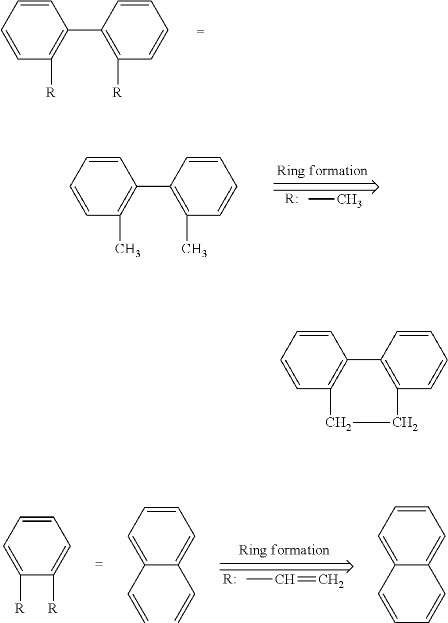

Furthermore, however, the above-mentioned formulation is also intended to be taken to mean that, in the case where one of the two radicals represents hydrogen, the second radical is bonded at the position to which the hydrogen atom was bonded, with formation of a ring. This is illustrated by the following scheme:

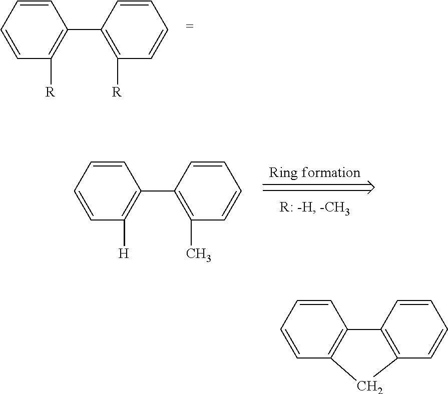

A sensitizer in the sense of the present invention is taken to mean a compound (donor), from which an energy transfer to another compound (acceptor) takes place.

According to the present invention, the electronic device comprise a sensitizer compound selected from compounds that exhibits delayed fluorescence or phosphorescent compounds.

Compounds exhibiting delayed fluorescence are preferably compounds which exhibit thermally activated delayed fluorescence. These compounds are abbreviated in the description which follows to “TADF compounds”.

As mentioned above, TADF compounds are compounds in which the energy gap between the lowest triplet state T₁ and the first excited singlet state S₁ is sufficiently small that the S₁ state is thermally accessible from the T₁ state.

Preferably, TADF compounds have a gap between the lowest triplet state T₁ and the first excited singlet state S₁ of 0.30 eV. More preferably, the gap between S₁ and T₁ is ≤0.20 eV, even more preferably ≤0.15 eV, especially more preferably ≤0.10 eV and even more especially preferably ≤0.08 eV. The energy of the lowest excited singlet state (S₁) and the lowest triplet state (T₁) are determined by quantum-chemical calculation.

A phosphorescent compound suitable as a sensitizer according to the invention can be any phosphorescent compound as long as the inter-system crossing rates are fast enough. One skilled in the art will have no difficulty in selecting from a variety of suitable compounds known to him the appropriate compounds for the present purpose. More particularly, a phosphorescent compound in the context of the present invention is a compound which is capable of emitting light at room temperature under optical or electrochemical excitation in an environment such as in an organic electroluminescent device, the emission being produced from a spin-forbidden transition, for example, a transition from an excited triplet state or a mixed singlet/triplet state.

Suitable phosphorescent compounds (=triplet emitters) are in particular compounds which emit light with suitable excitation, preferably in the visible range, and also at least one atom of atomic number greater than 20, preferably greater than 38 and less than 84, particularly preferably greater than 56 and smaller than 80, in particular a metal with this atomic number.

Preferably, the sensitizer is a phosphorescent compound selected from the group of the organometallic complexes, particularly from the group of the transition metal complexes.

Very preferably, the sensitizer is a phosphorescent compound, selected from organometallic complexes containing copper, molybdenum, tungsten, rhenium, ruthenium, osmium, rhodium, iridium, palladium, platinum, silver, gold or europium, particularly organometallic complexes containing copper, iridium or platinum, and very particularly organometallic complexes containing Iridium and platinum. For the purposes of the present invention, all luminescent compounds which contain the abovementioned metals are regarded as phosphorescent compounds.

Particularly preferred are the phosphorescent organometallic complexes, which are described, for example, in WO2015/091716. Also particularly preferred are the phosphorescent organometallic complexes, which are described in WO2000/70655, WO2001/41512, WO2002/02714, WO2002/15645, EP1191612, WO2005/033244, WO2005/019373, US2005/0258742, WO2006/056418, WO2007/115970, WO2007/115981, WO2008/000727, WO2009/050281, WO2009/050290, WO2011/051404, WO2011/073149, WO2012/121936, US2012/0305894, WO2012/170571, WO2012/170461, WO2012/170463, WO2006/121811, WO2007/095118, WO2008/156879, WO2008/156879, WO2010/068876, WO2011/106344, WO2012/172482, EP3126371, WO2015/014835, WO2015/014944, WO2016/020516, US20160072081, WO2010/086089, WO2011/044988, WO2014/008982, WO2014/023377, WO2014/094961, WO2010/069442, WO2012/163471, WO2013/020631, US20150243912, WO2008/000726, WO2010/015307, WO2010/054731, WO2010/054728, WO2010/099852, WO2011/032626, WO2011/157339, WO2012/007086, WO2015/036074, WO2015/104045, WO2015/117718, WO2016/015815, which are preferably iridium and platinum complexes.

Particularly preferred are also the phosphorescent organometallic complexes having polypodal ligands as described, for example, in WO2004/081017, WO2005/042550, US2005/0170206, WO2009/146770, WO2010/102709, WO2011/066898, WO2016124304, WO2017/032439, WO2018/019688, EP3184534 and WO2018/011186.

Particularly preferred are also the phosphorescent binuclear organometallic complexes as described, for example, in WO2011/045337, US20150171350, WO2016/079169, WO2018/019687, WO2018/041769, WO2018/054798, WO2018/069196, WO2018/069197, WO2018/069273.

Particularly preferred are also the copper complexes as described, for example, in WO2010/031485, US2013150581, WO2013/017675, WO2013/007707, WO2013/001086, WO2012/156378, WO2013/072508, EP2543672.

In general, all phosphorescent complexes, which are used according to the prior art for phosphorescent OLEDs and which are known to the person skilled in the art in the field of organic electroluminescence, are suitable. The person skilled in the art can use further phosphorescent complexes without any inventive step.

In a preferred embodiment of the invention, the emitting layer is produced by vapor deposition and the phosphorescent compound is present in a doping concentration of 5 to 99.9% by volume in the emitting layer, preferably from 5 to 60% by volume, very preferably from 10 to 50% by volume, most preferably from 20 to 40% by volume.

In another preferred embodiment of the invention, the emitting layer is produced via a solution process and the phosphorescent compound is present in a doping concentration of 5 to 99.9% by weight in the emitting layer, preferably from 5 to 60% by weight, particularly preferably from 10 to 50% by weight, most preferably 20 to 40% by weight.

Explicit examples of phosphorescent sensitizers are Ir(ppy)₃ and its derivatives as well as the structures listed below:

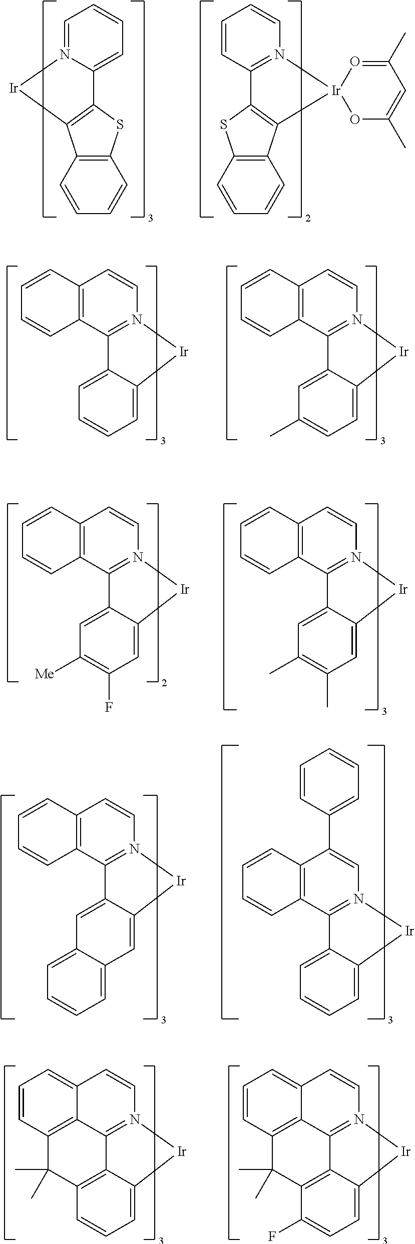

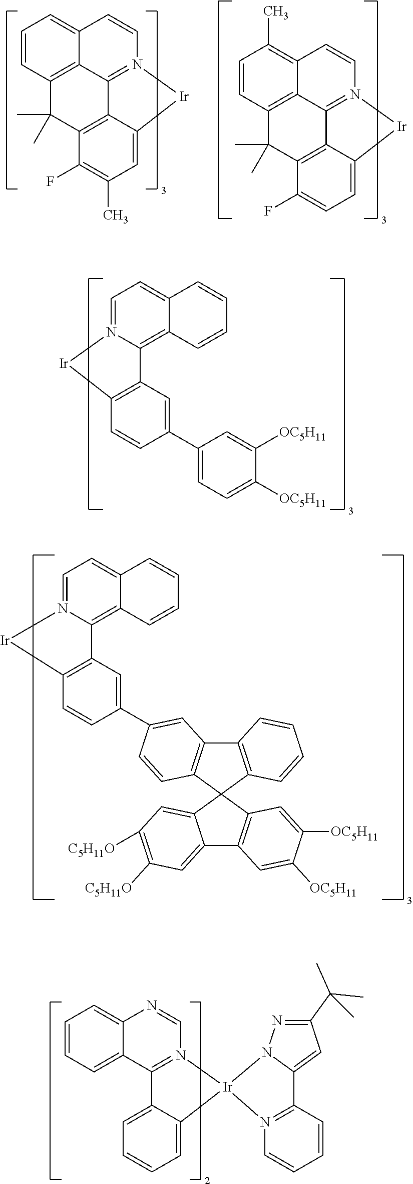

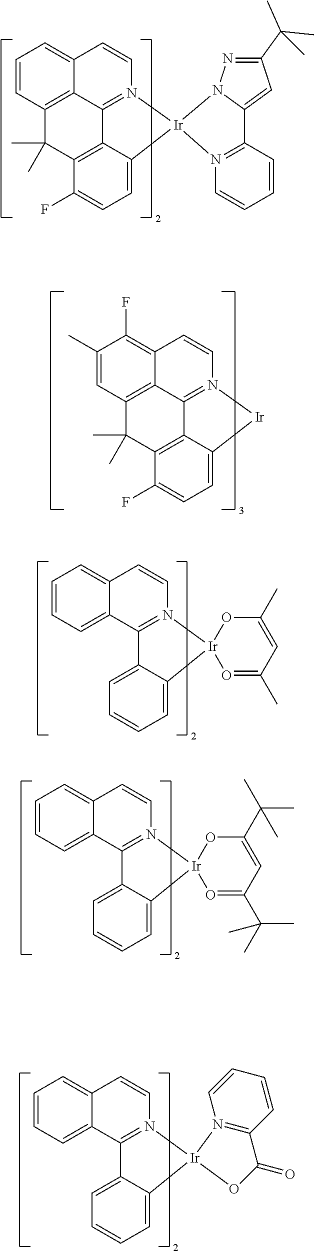

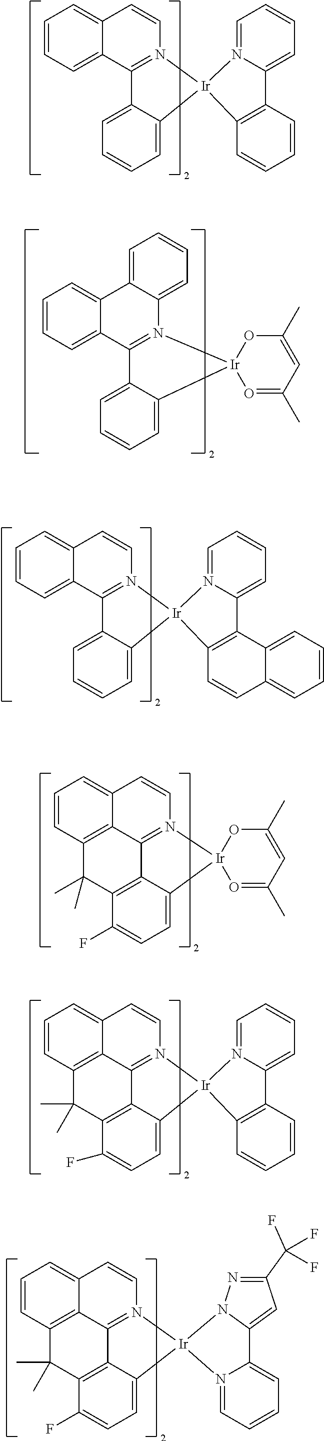

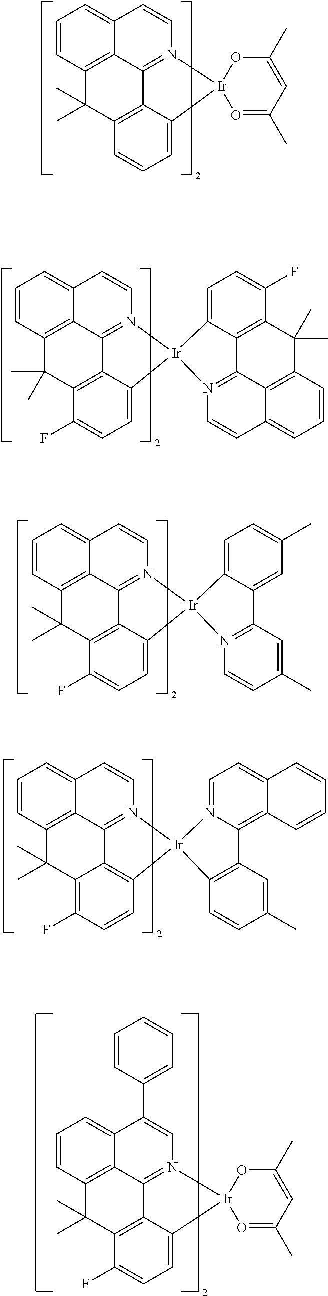

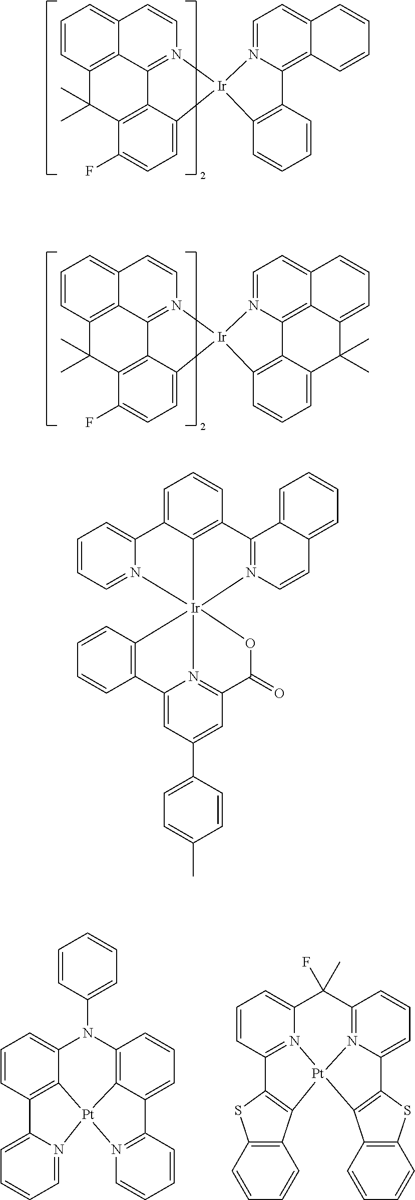

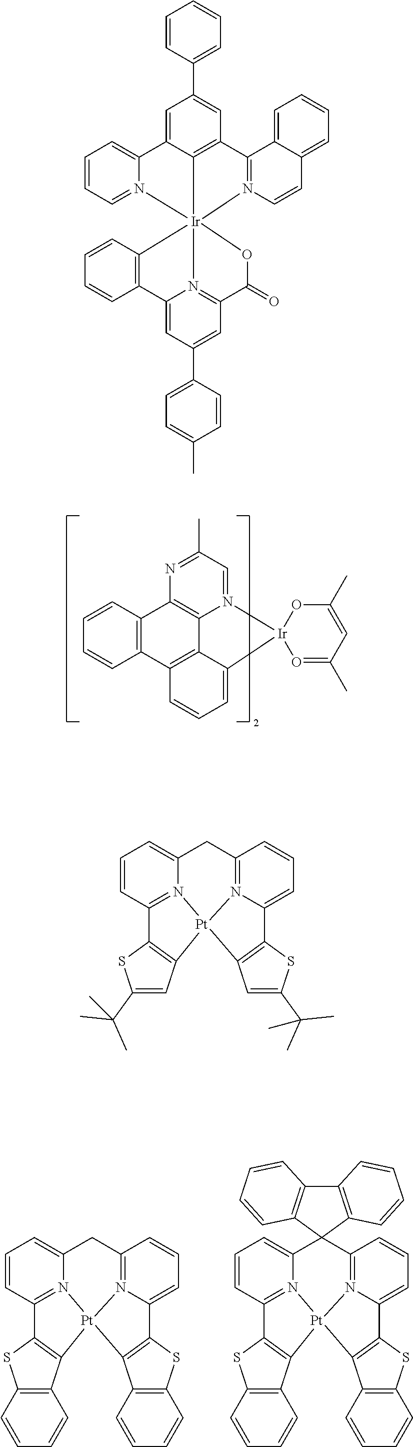

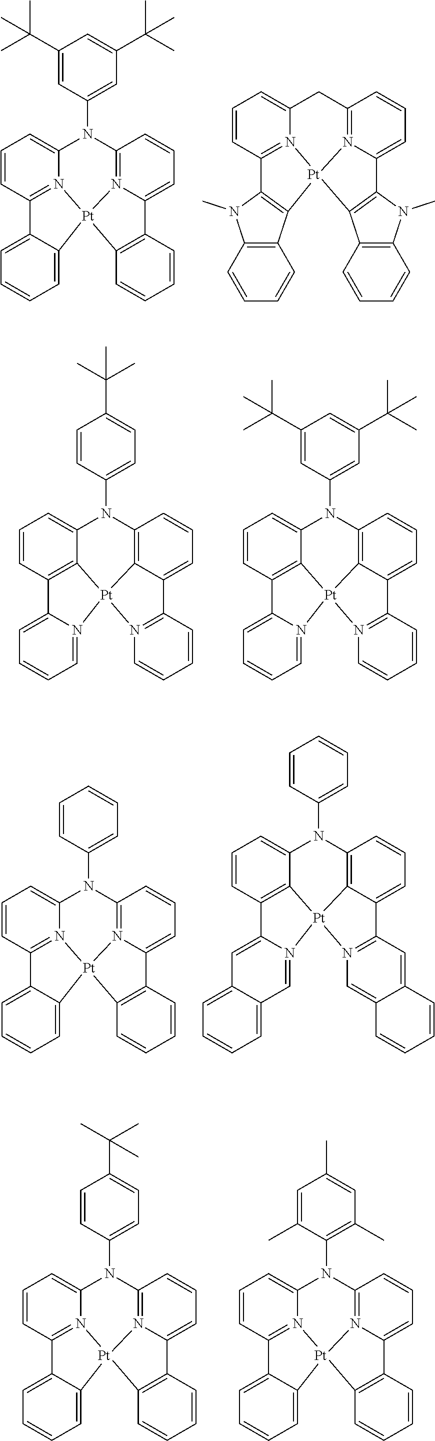

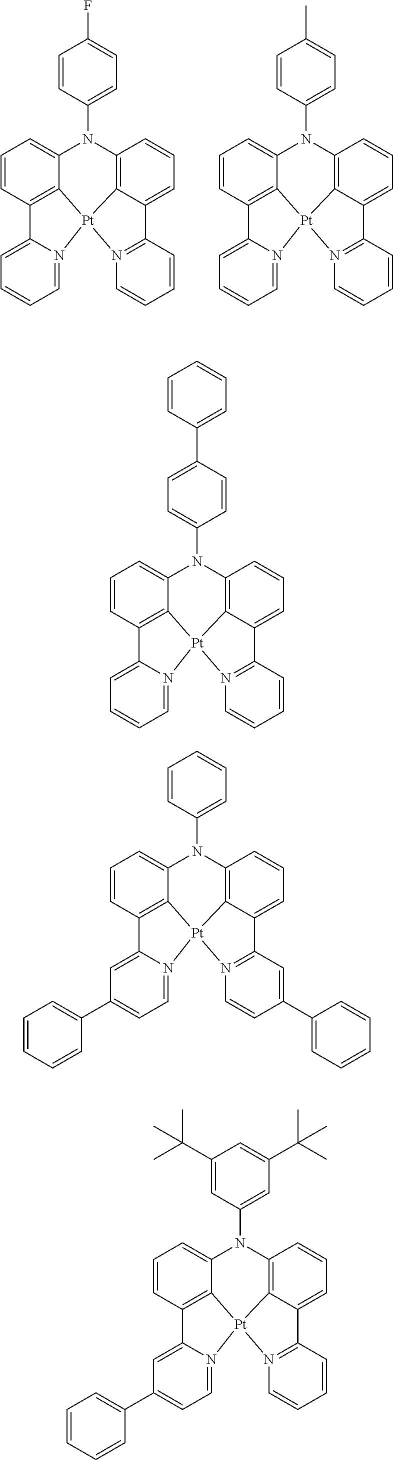

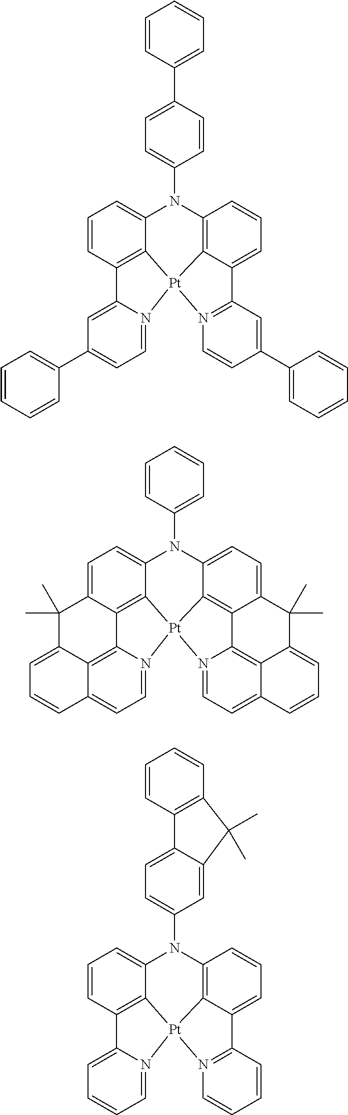

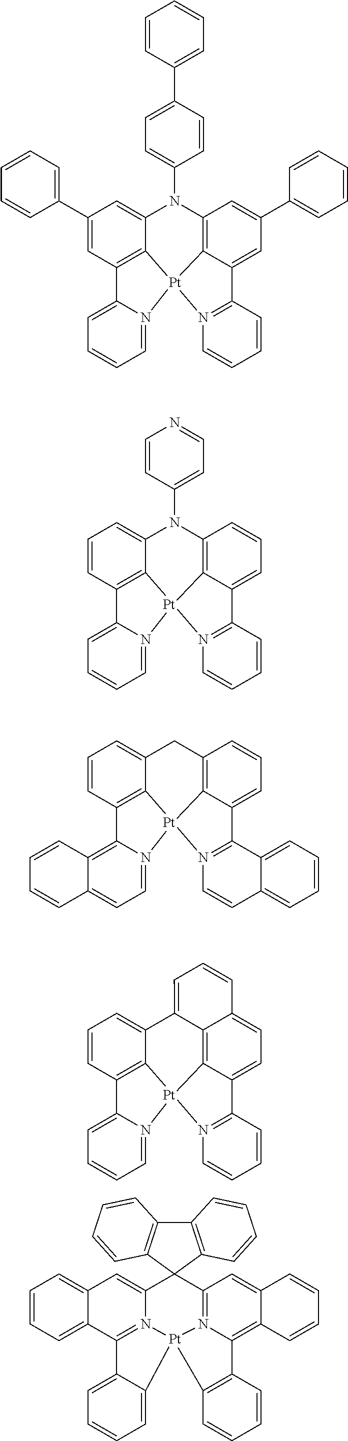

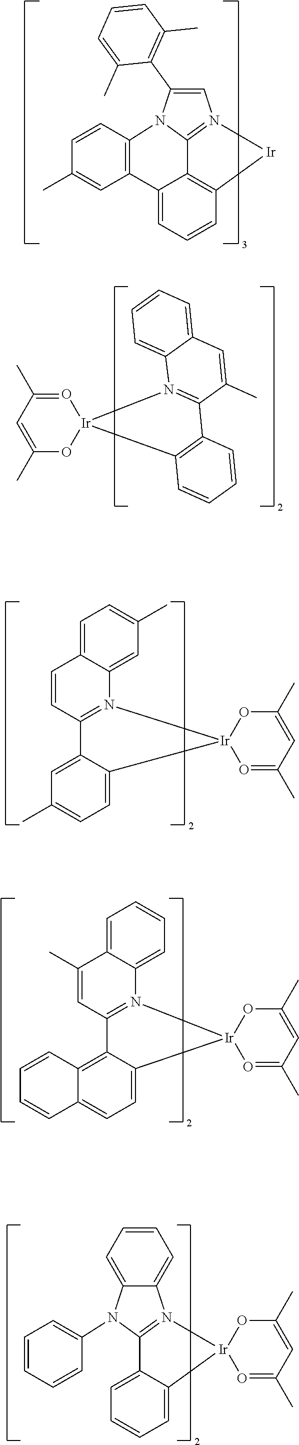

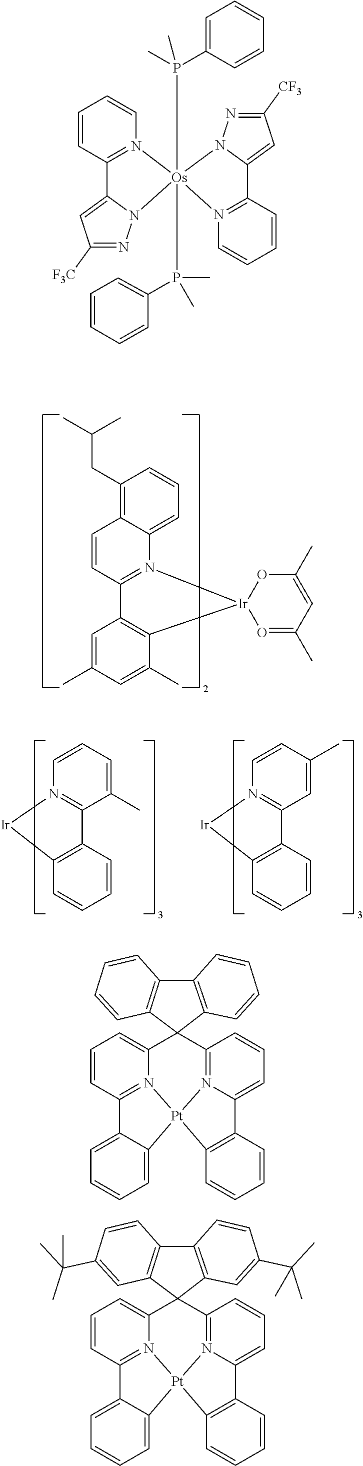

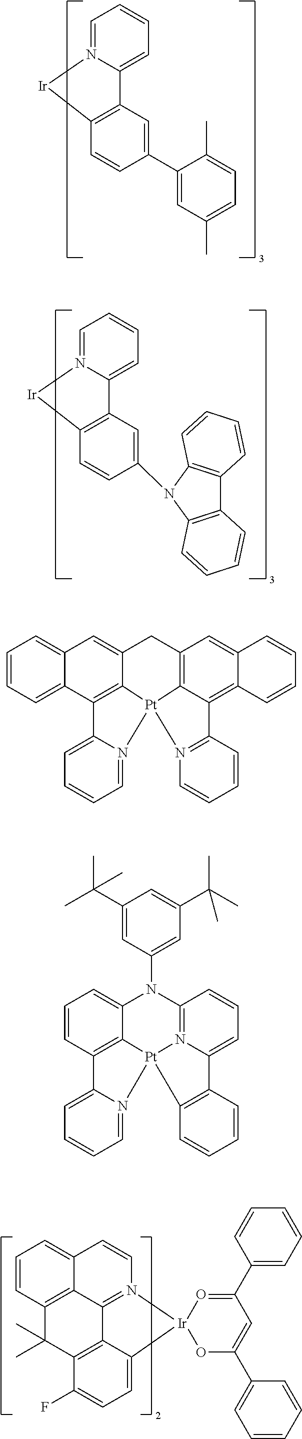

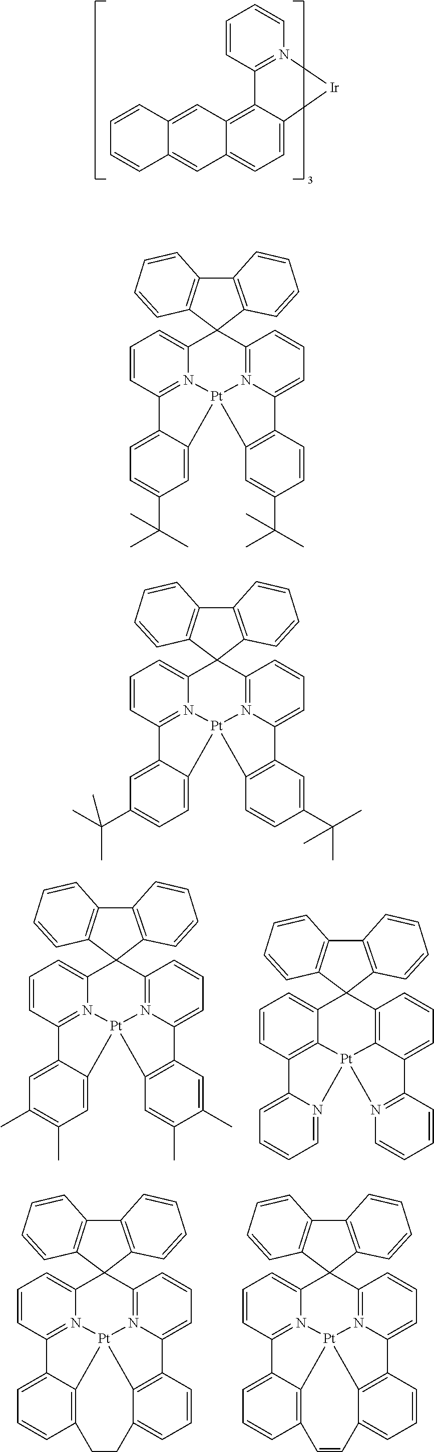

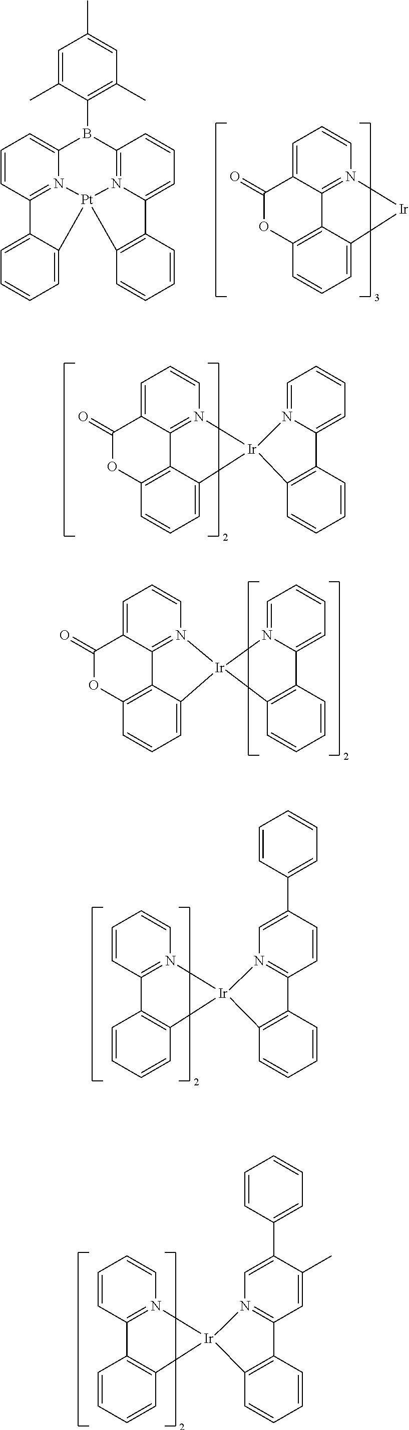

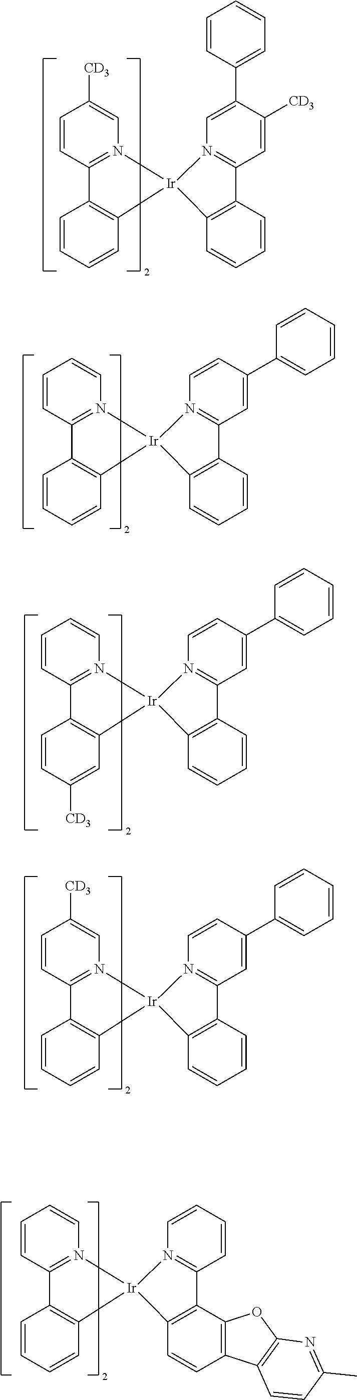

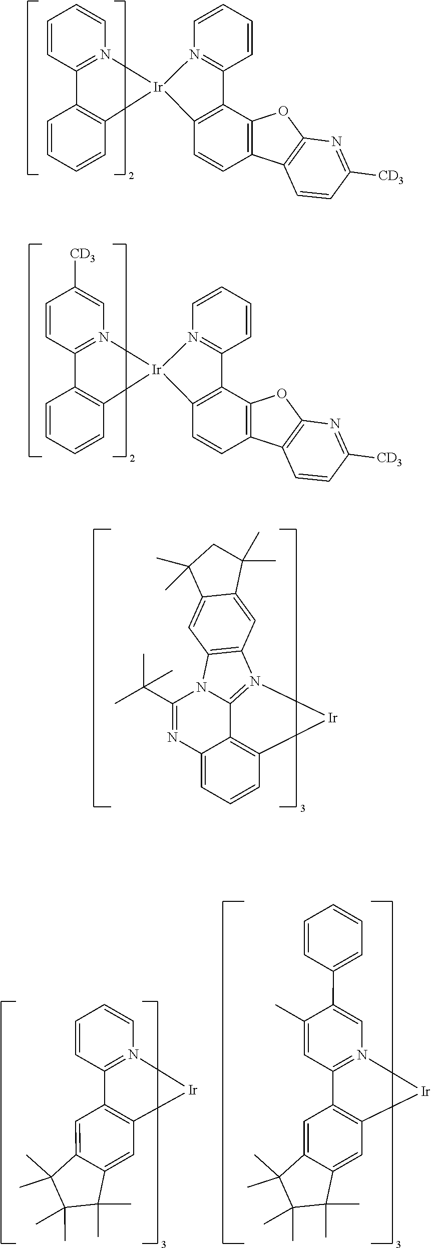

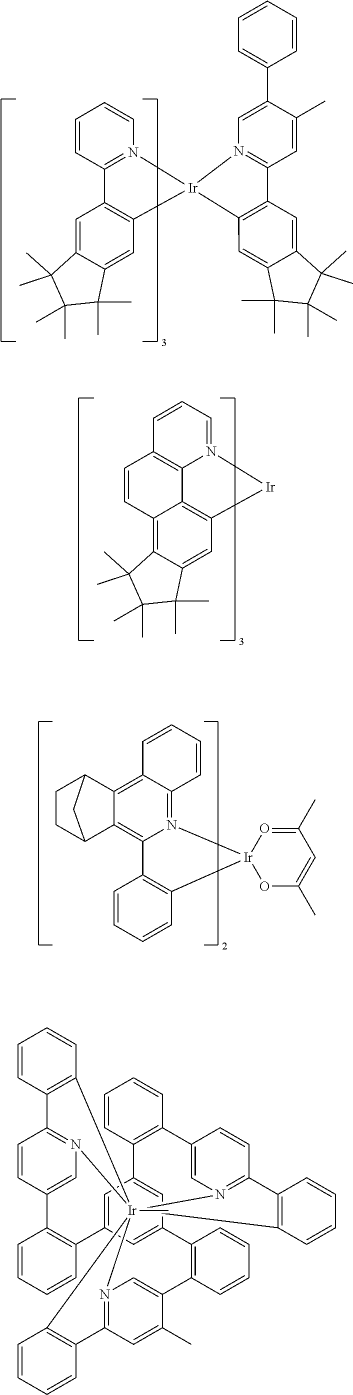

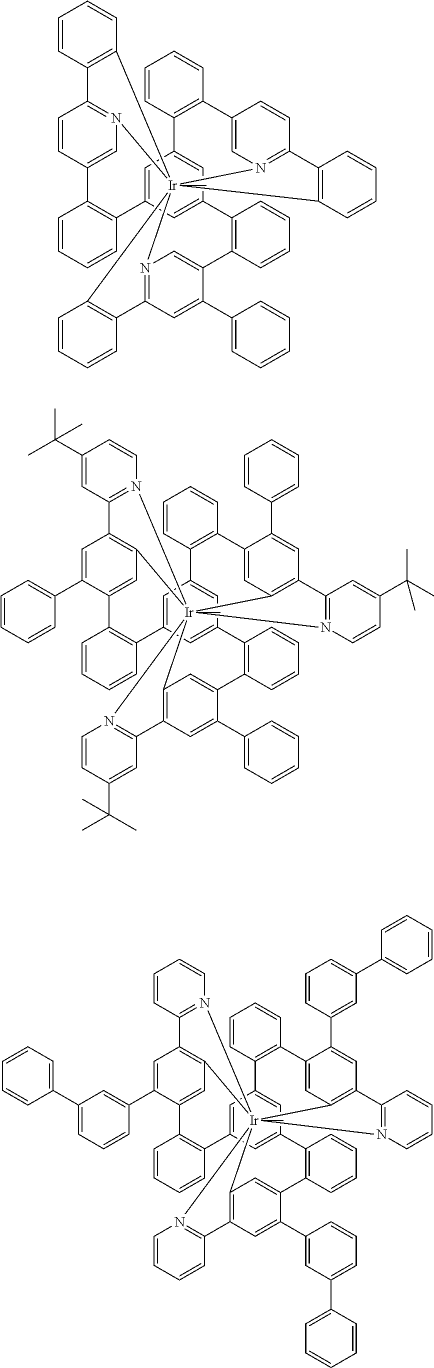

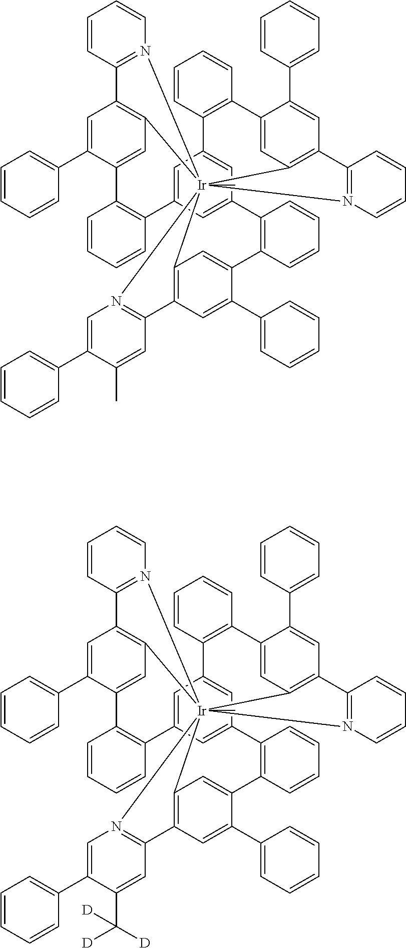

Further explicit examples of phosphorescent sensitizers are iridium and platinum complexes containing carbene ligands and the structures listed below, wherein homoleptic and heteroleptic complexes and meridonal and facial isomers may be suitable:

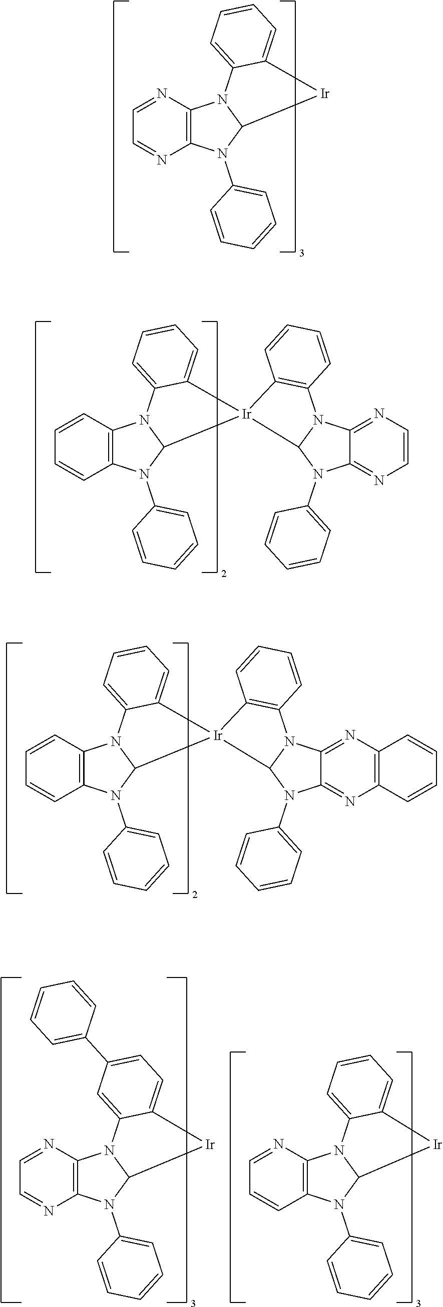

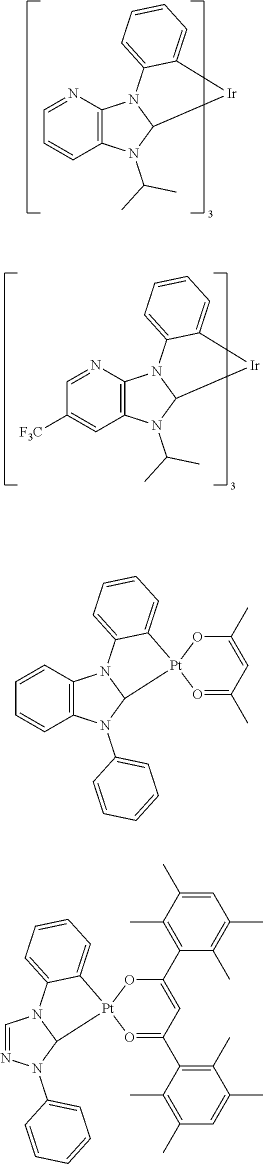

Further explicit examples of phosphorescent sensitizers are also copper complexes and the structures listed below:

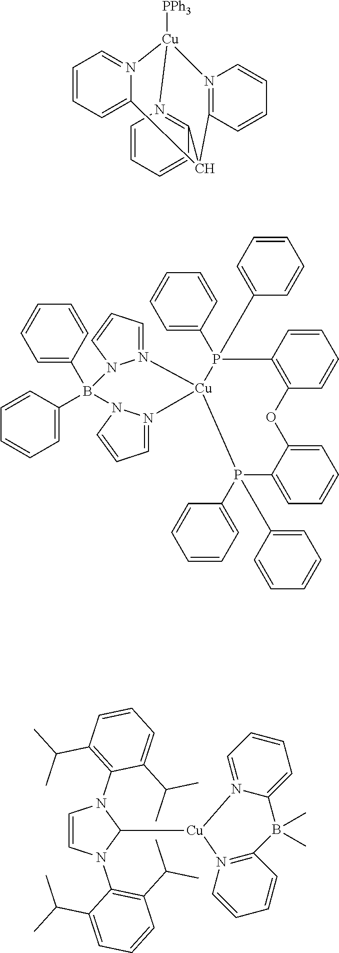

In accordance with the invention, the electronic device comprises a sterically hindered fluorescent perylene emitter compound of formula (I) as described above.

The steric shielding of the perylene emitter is accomplished by electronically inert, sterically demanding substituents among R¹ to R¹² in formula (I), which surround the electronically active perylene core of the fluorescent compound and thus shield it substantially from contact with adjacent molecules in the layer.

Suitable sterically demanding substituents are, for example, alkyl groups, especially having 3 to 20 carbon atoms, preferably having 4 to 10 carbon atoms, in which hydrogen atoms may also be replaced by F, alkoxy groups, especially having 3 to 20 carbon atoms, preferably having 4 to 10 carbon atoms, aralkyl groups, especially having 7 to 30 carbon atoms, and aromatic ring systems, especially having 6 to 30 carbon atoms, where it is also possible for the aryl groups in the aralkyl groups and aromatic ring systems to be substituted by one or more alkyl groups having 1 to 10 carbon atoms. It is also possible here for a plurality of adjacent substituents to form a ring system with one another.

When the substituent is an aralkyl group or an aromatic ring system, it is preferable when these do not have any fused aryl groups having more than 10 carbon atoms in which aryl groups are fused directly to one another via a common edge. More preferably, it does not have any fused aryl groups at all in which aryl groups are fused directly to one another via a common edge. Thus, it is preferable when the aromatic ring system, for example, does not have any anthracene or pyrene groups, and particularly preferable when the aromatic ring system does not have any naphthalene groups either. By contrast, it may have, for example, biphenyl or terphenyl groups, since these do not have any fused aryl groups. In addition, it may also have, for example, fluorene or spirobifluorene groups, since no aryl groups are fused directly to one another via a common edge in these groups.

When the sterically demanding substituent is an alkyl group, this alkyl group preferably has 4 to 10 carbon atoms. Preference is given to a secondary, tertiary or cyclic alkyl group in which the secondary or tertiary carbon atom is either bonded to the fluorescent base skeleton directly or bonded to the fluorescent base skeleton via a CH₂ group. More preferably, this alkyl group is selected from the structures of the following formulae (RS-1) to (RS-33):

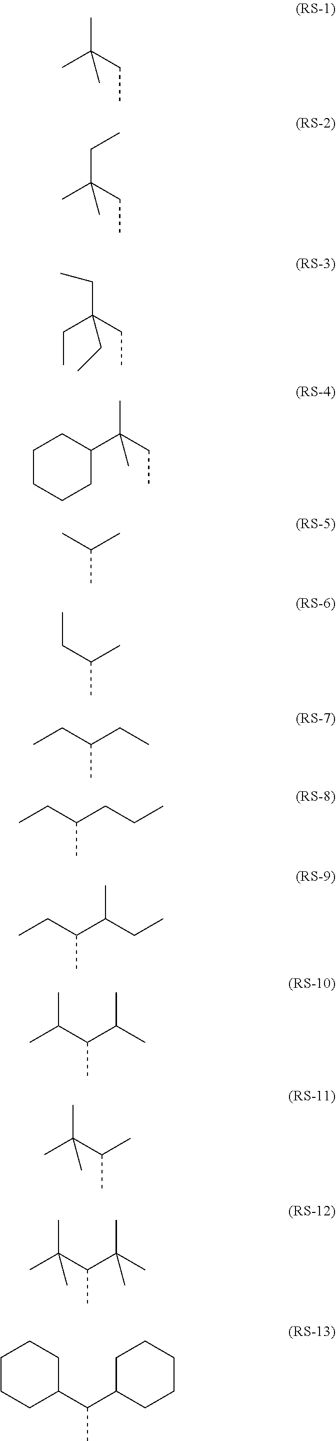

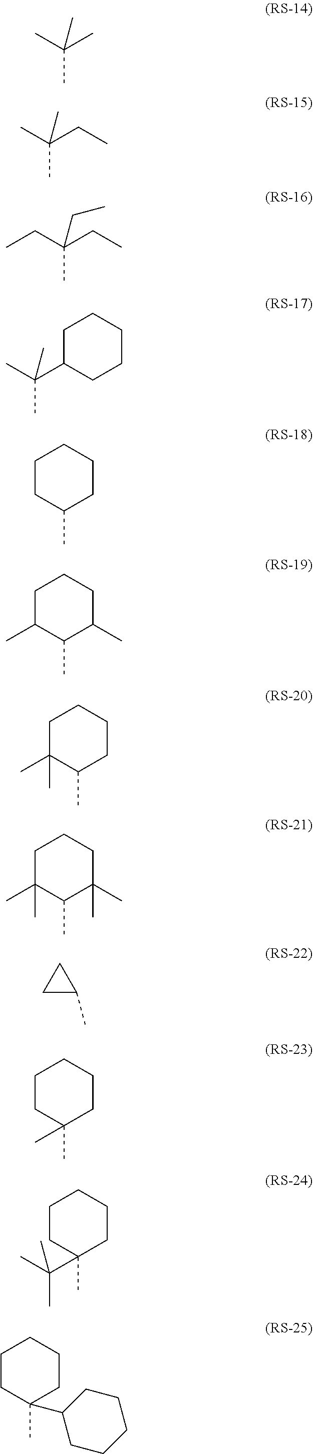

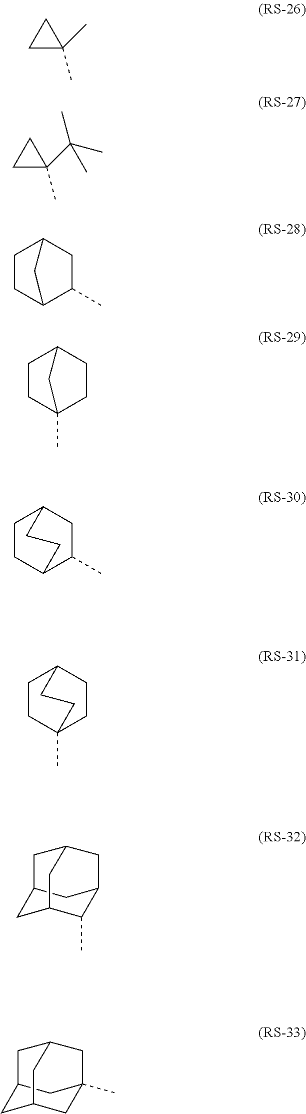

where the dotted bond indicates the linkage of these groups to the perylene base skeleton.

When the sterically demanding substituent is an alkoxy group, this alkoxy group preferably has 3 to 10 carbon atoms and is preferably branched or cyclic. Preferably, this alkoxy group is selected from the structures of the following formulae (RS-34) to (RS-47):

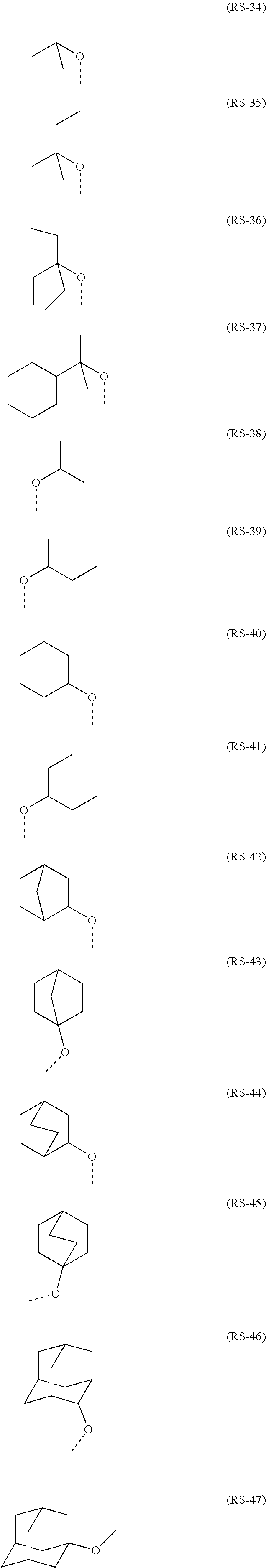

where the dotted bond indicates the linkage of these groups to the perylene base skeleton.

When the sterically demanding substituent is an aralkyl group, this aralkyl group is preferably selected from the structures of the following formulae (RS-48) to (RS-61):

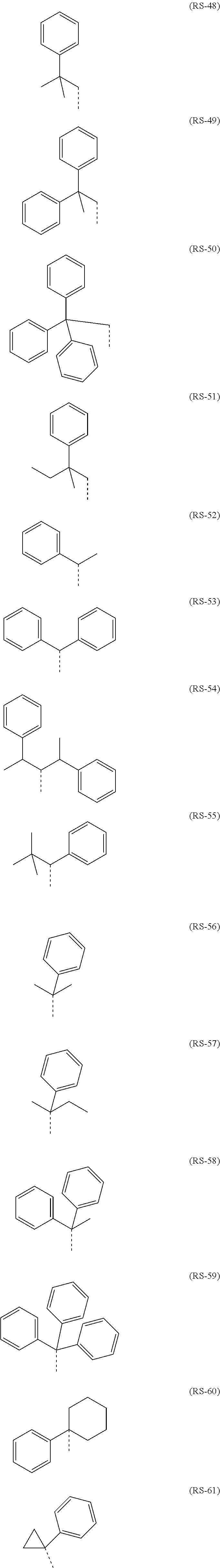

where the dotted bond indicates the linkage of these groups to the perylene base skeleton and the phenyl groups may each be substituted by one or more R^{a} radicals, where:
R^{a} is the same or different at each instance and is selected from the group consisting of H, D, F, a straight-chain alkyl group having 1 to 40 carbon atoms or a branched or cyclic alkyl group having 3 to 40 carbon atoms, each of which may be substituted by one or more R^{b} radicals, an aromatic ring system having 5 to 60 aromatic ring atoms, each of which may be substituted by one or more R^{b} radicals, or an aralkyl group which has 5 to 60 aromatic ring atoms and may be substituted by one or more R^{b} radicals, where it is optionally possible for two or more adjacent R^{a} substituents to form a ring system which may be substituted by one or more R^{b} radicals;
R^{b} is selected from the group consisting of H, D, F, an aliphatic hydrocarbyl radical having 1 to 20 carbon atoms, an aromatic ring system having 5 to 30 aromatic ring atoms, where two or more adjacent R^{b} substituents together may form a ring system.

When the sterically demanding substituent is an aromatic ring system, this aromatic ring system preferably has 6 to 30 aromatic ring atoms, more preferably 6 to 24 aromatic ring atoms. In addition, this aromatic ring system contains preferably only phenyl groups. In this case, the aromatic ring system is preferably selected from the structures of the following formulae (RS-62) to (RS-76):

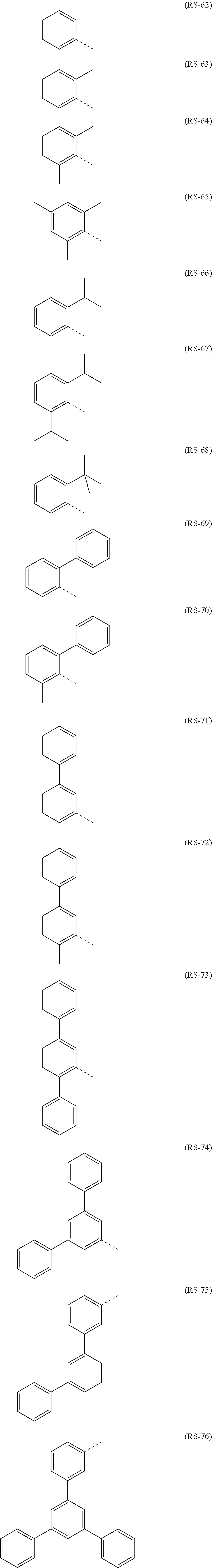

where the dotted bond indicates the linkage of these groups to the perylene base skeleton and the phenyl groups may each be substituted by one or more R^{a} radicals as defined above.

Preferably, the electronic device comprises a sterically hindered fluorescent perylene emitter of formula (I), selected from compounds of formula (II):

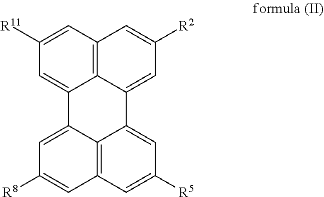

wherein
R², R⁵, R⁸, R¹¹ are each selected, identically or differently, from a straight-chain alkyl or alkoxy group having 3 to 20 carbon atoms, a branched or cyclic alkyl or alkoxy group having 3 to 20 carbon atoms, an alkenyl or alkynyl group having 3 to 20 carbon atoms, an aralkyl group, preferably having 7 to 60 carbon atoms, where the above-mentioned groups may each be substituted by one or more radicals R²⁰ and where one or more CH₂ groups in the above-mentioned groups may be replaced by Si(R²⁰)₂, Ge(R²⁰)₂, Sn(R²⁰)₂, C═O, C═S, C═Se, C═NR²⁰, P(═O)(R²⁰), SO, SO₂, NR²⁰, —O—, —S—, —COO— or —CONR²⁰— and where one or more H atoms in the above-mentioned groups may be replaced by D, F, Cl, Br, I, CN or NO₂, or an aromatic ring system having 5 to 60 aromatic ring atoms, which may in each case be substituted by one or more radicals R²⁰;
R²⁰ is on each occurrence, identically or differently, selected from H, D, F, or a straight-chain alkyl group having 1 to 40 carbon atoms, or a branched or cyclic alkyl group having 3 to 40 carbon atoms, or an alkenyl or alkynyl group having 2 to 40 carbon atoms, or an aralkyl group having 7 to 40 carbon atoms, where the above-mentioned groups may each be substituted by one or more radicals R²¹, or an aromatic ring system having 5 to 40 aromatic ring atoms, which may in each case be substituted by one or more radicals R²¹, where two or more radicals R²⁰ may be joined to form an aromatic ring system or a (poly)cyclic alkyl group, which may in each case be substituted by one or more radicals R²¹;
R²¹ is on each occurrence, identically or differently, selected from H, D, F, or a straight-chain alkyl group having 1 to 20 carbon atoms, or a branched or cyclic alkyl group having 3 to 20 carbon atoms, or an alkenyl or alkynyl group having 2 to 20 carbon atoms, or an aromatic ring system having 5 to 40 aromatic ring atoms, where two or more radicals R²¹ may be joined to form an aromatic ring system or a (poly)cyclic alkyl group.

More preferably, the electronic device comprises a sterically hindered fluorescent perylene emitter compound of formula (I) or (II), where:
R², R⁵, R⁸, R¹¹ are each selected, identically or differently, from a straight-chain, branched or cyclic alkyl group having 4 to 10 carbon atoms, a straight-chain, branched or cyclic alkoxy group having 3 to 10 carbon atoms, an aralkyl group having 7 to 30 carbon atoms, where the above-mentioned groups may each be substituted by one or more radicals R²⁰ and where one or more H atoms in the above-mentioned groups may be replaced by D, F, Cl or CN, or an aromatic ring system having 6 to 30 aromatic ring atoms, which may in each case be substituted by one or more radicals R²⁰;
R²⁰ is on each occurrence, identically or differently, selected from D, F, or a straight-chain alkyl group having 1 to 20 carbon atoms or a branched or cyclic alkyl group having 3 to 20 carbon atoms or an alkenyl or alkynyl group having 2 to 20 carbon atoms, where the above-mentioned groups may each be substituted by one or more radicals R²¹, or an aromatic ring system having 5 to 30 aromatic ring atoms, which may in each case be substituted by one or more radicals R²¹, where two or more radicals R²⁰ may be joined to form an aromatic ring system or a (poly)cyclic alkyl group, which may in each case be substituted by one or more radicals R²¹;
R²¹ is on each occurrence, identically or differently, selected from H, D, F, or a straight-chain alkyl group having 1 to 10 carbon atoms, or a branched or cyclic alkyl group having 3 to 10 carbon atoms, or an alkenyl or alkynyl group having 2 to 10 carbon atoms, or an aromatic ring system having 5 to 30 aromatic ring atoms, where two or more radicals R²¹ may be joined to form an aromatic ring system or a (poly)cyclic alkyl group.

Even more preferably, the electronic device comprises a sterically hindered fluorescent perylene emitter compound of formula (I), selected from compounds of formula (II), where:
R², R⁵, R⁸, R¹¹ are each selected, identically or differently, from branched or cyclic alkyl groups represented by the general following formula (R-a)

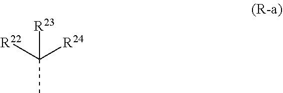

wherein
R²², R²³, R²⁴ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R²⁵, and where two of radicals R²², R²³, R²⁴ or all radicals R²², R²³, R²⁴ may be joined to form a (poly)cyclic alkyl group, which may be substituted by one or more radicals R²⁵;
R²⁵ is at each occurrence, identically or differently, selected from a straight-chain alkyl group having 1 to 10 carbon atoms, or a branched or cyclic alkyl group having 3 to 10 carbon atoms;
with the proviso that at each occurrence at least one of radicals R²², R²³ and R²⁴ is other than H, with the proviso that at each occurrence all of radicals R²², R²³ and R²⁴ together have at least 4 carbon atoms and with the proviso that at each occurrence, if two of radicals R²², R²³, R²⁴ are H, the remaining radical is not a straight-chain;
or form branched or cyclic alkoxy groups represented by the general following formula (R-b)

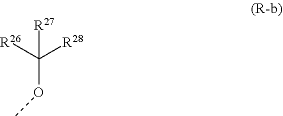

wherein
R²⁶, R²⁷, R²⁸ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R²⁵ as defined above, and where two of radicals R²⁶, R²⁷, R²⁸ or all radicals R²⁶, R²⁷, R²⁸ may be joined to form a (poly)cyclic alkyl group, which may be substituted by one or more radicals R²⁵ as defined above;
with the proviso that at each occurrence only one of radicals R²⁶, R²⁷ and R²⁸ may be H;
or from aralkyl groups represented by the general following formula (R-c)

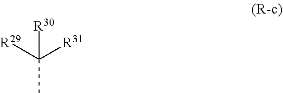

wherein
R²⁹, R³⁰, R³¹ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 30 aromatic ring atoms, which may in each case be substituted by one or more radicals R³², and where two or all of radicals R²⁹, R³⁰, R³¹ may be joined to form a (poly)cyclic alkyl group or an aromatic ring system, each of which may be substituted by one or more radicals R³²;
R³² is at each occurrence, identically or differently, selected from a straight-chain alkyl group having 1 to 10 carbon atoms, or a branched or cyclic alkyl group having 3 to 10 carbon atoms, or an aromatic ring system having 6 to 24 aromatic ring atoms;
with the proviso that at each occurrence at least one of radicals R²⁹, R³⁰ and R³¹ is other than H and that at each occurrence at least one of radicals R²⁹, R³⁰ and R³¹ is or contains an aromatic ring system having at least 6 aromatic ring atoms;
or from aromatic ring systems represented by the general following formula (R-d)

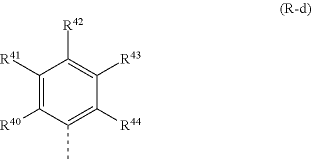

wherein
R⁴⁰ to R⁴⁴ is at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³² as defined above, or an aromatic ring system having 6 to 30 aromatic ring atoms, which may in each case be substituted by one or more radicals R³² as defined above, and where two or more of radicals R⁴⁰ to R⁴⁴ may be joined to form a (poly)cyclic alkyl group or an aromatic ring system, each of which may be substituted by one or more radicals R³² as defined above.

Particularly preferably, the electronic device comprises a sterically hindered fluorescent perylene emitter compound of formula (I) or (II), where the groups R², R⁵, R⁸, R¹¹ are identical.

In accordance with a preferred embodiment, the electronic device comprises a sterically hindered fluorescent perylene emitter compound of formula (I), selected from compounds of formula (III) or (IV)

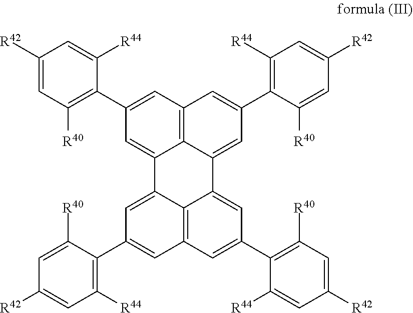

wherein
R⁴⁰, R⁴², R⁴⁴ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 30 aromatic ring atoms, which may in each case be substituted by one or more radicals R³²;
   with the proviso that at least one of R⁴⁰, R⁴², R⁴⁴ is other than H; or

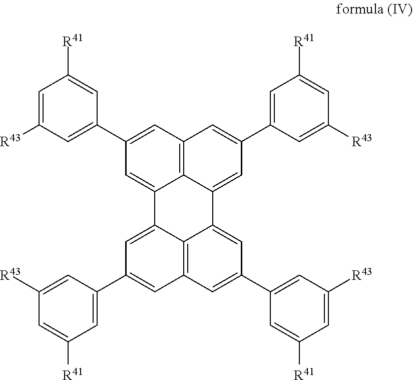

wherein
R⁴¹, R⁴³ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 30 aromatic ring atoms, which may in each case be substituted by one or more radicals R³²;
   with the proviso that at least one of R⁴¹, R⁴³ is other than H.

Preferably, the groups R⁴², R⁴⁰ and R⁴⁴ in the compounds of formula (III) are defined as follows:
R⁴² is at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³²;
R⁴⁰, R⁴⁴ are at each occurrence, identically or differently, selected from an aromatic ring system having 6 to 30 aromatic ring atoms, which may in each case be substituted by one or more radicals R³²; where R³² is as defined as above.

In accordance with a preferred embodiment, the groups R⁴², R⁴⁰ and R⁴⁴ are at each occurrence, identically or differently, selected from an aromatic ring system having 6 to 30 aromatic ring atoms, which may in each case be substituted by one or more radicals R³².

In accordance with another preferred embodiment, the group R⁴² is at each occurrence, identically or differently, selected from H, a straight-chain alkyl group having 1 to 10 carbon atoms, or a branched or cyclic alkyl group having 3 to 10 carbon atoms, or an aromatic ring system having 6 to 30 aromatic ring atoms, which may in each case be substituted by one or more radicals R³², and the R⁴⁰, R⁴⁴ are at each occurrence identically selected from a straight-chain alkyl group having 1 to 10 carbon atoms, or a branched or cyclic alkyl group having 3 to 10 carbon atoms, which may in each case be substituted by one or more radicals R³².

In accordance with a very preferred embodiment, the electronic device comprises a sterically hindered fluorescent perylene emitter compound of formula (I), selected from a compound of one of the formulae (IIIa), (IIIb) or (IIIc)

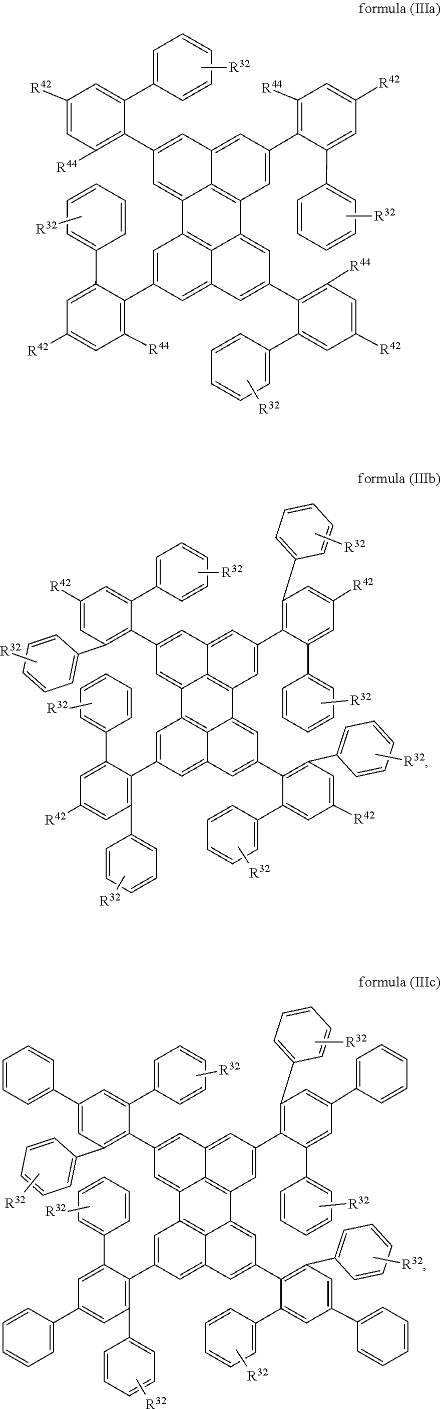

wherein in each of formulae (IIIa), (IIIb) and (IIIc) the phenyl groups indicated with —R³² are unsubstituted or substituted with one or more radicals R³²;
R⁴² and R⁴⁴ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³²; where R³² is as defined as above.

In one embodiment of the invention, the electronic device comprises an organic layer comprising a mixture of the sterically shielded fluorescent perylene emitter compound and of the sensitizer compound.

In a further embodiment of the invention, the electroluminescent device comprises, adjoining the organic layer comprising the sterically shielded fluorescent perylene emitter compound, a layer comprising the sensitizer compound on the anode side.

In a further embodiment of the invention, the electroluminescent device comprises, adjoining the organic comprising the sterically shielded fluorescent perylene emitter compound, a layer comprising the sensitizer compound on the cathode side.

Preferably, the organic layer comprises the sterically shielded fluorescent perylene emitter and the sensitizer compound, and the organic layer is more preferably an emitting layer.

Because of the difference in production of the organic electroluminescent device, the dopant concentration of the shielded perylene compound in the case of production of the emitting layer by vapor deposition is reported in % by volume, and in the case of production of the emitting layer from solution in % by weight.

In a preferred embodiment of the invention, in the case of production of the emitting layer by vapor deposition, the shielded perylene compound is present in a dopant concentration of 0.1% to 25% by volume in the emitting layer, preferably of 1% to 20% by volume, more preferably of 2% to 12% by volume, even more preferably 3% to 10% by volume.

In a preferred embodiment of the invention, in the case of production of the emitting layer from solution, the shielded perylene compound is present in a dopant concentration of 0.1% to 25% by weight in the emitting layer, preferably of 1% to 20% by weight, more preferably of 2% to 12% by weight, even more preferably 3% to 10% by weight.

It is possible here that, especially in the case of a low dopant concentration of the shielded perylene compound, the OLED exhibits mixed emission composed of the fluorescent compound and residual emission of the sensitizer compound. This can also be utilized in a controlled manner to generate mixed colors.

In accordance with a preferred embodiment, the electronic device comprises an organic layer comprising the sterically hindered fluorescent emitter compound, the sensitizer compound and at least one organic functional material selected from the group consisting of HTM, HIM, HBM, p-dopant, ETM, EIM, EBM, n-dopant, fluorescent emitter, phosphorescent emitter, delayed fluorescent material, matrix material, host material, wide band gap material, quantum material (preferably quantum dot), said organic layer preferably being the emitting layer. Preferably, the at least one organic functional material is selected from matrix materials. This further compound is referred to hereinafter as matrix compound or matrix material. This may be a further sensitizer compound in the context of the definition detailed above. In general, the matrix compound, however, is not a sensitizer compound.

In a preferred embodiment of the invention, the matrix compound makes no significant contribution, if any, to the emission of the mixture.

It is preferable that the lowest triplet energy of the matrix compound is not more than 0.1 eV lower than the triplet energy of the sensitizer compound.

Especially preferably, T₁ (matrix)≥T₁(sensitizer).

More preferably: T₁(matrix)−T₁(sensitizer)≥0.1 eV;

most preferably: T₁(matrix)−T₁(sensitizer)≥0.2 eV.

T₁(matrix) here is the lowest triplet energy of the matrix compound and T₁(sensitizer) is the lowest triplet energy of the sensitizer compound. The triplet energy of the matrix compound T₁(matrix) is determined here from the edge of the photoluminescence spectrum measured at 4 K of the neat film. T₁(sensitizer) is determined from the edge of the photoluminescence spectrum measured at room temperature in toluene solution.”

Examples of suitable matrix compounds which can be used in the emitting layer of the invention are ketones, phosphine oxides, sulfoxides and sulfones, for example according to WO 2004/013080, WO 2004/093207, WO 2006/005627 or WO 2010/006680, triarylamines, carbazole derivatives, e.g. CBP (N,N-biscarbazolylbiphenyl), m-CBP or the carbazole derivatives disclosed in WO 2005/039246, US 2005/0069729, JP 2004/288381, EP 1205527, WO 2008/086851 or US 2009/0134784, dibenzofuran derivatives, indolocarbazole derivatives, for example according to WO 2007/063754 or WO 2008/056746, indenocarbazole derivatives, for example according to WO 2010/136109 or WO 2011/000455, azacarbazoles, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, diazasilole derivatives, for example according to WO 2010/054729, diazaphosphole derivatives, for example according to WO 2010/054730, triazine derivatives, for example according to WO 2010/015306, WO 2007/063754 or WO 2008/056746, pyrimidine derivatives, quinoxaline derivatives, Zn complexes, Al complexes or Be complexes, for example according to EP 652273 or WO 2009/062578, or bridged carbazole derivatives, for example according to US 2009/0136779, WO 2010/050778, WO 2011/042107 or WO 2011/088877, or spirodibenzopyranamines (for example in accordance with WO 2013/083216). Suitable matrix materials are also those described in WO 2015/135624. These are incorporated into the present invention by reference. It is also possible to use mixtures of two or more of these matrix materials.

Preferably, the matrix compound has a glass transition temperature TG of greater than 70° C., more preferably greater than 90° C., most preferably greater than 110° C.

The matrix compounds are preferably charge-transporting, i.e. electron-transporting or hole-transporting, or bipolar compounds. Matrix compounds used may additionally also be compounds which are neither hole- nor electron-transporting in the context of the present application.

An electron-transporting compound in the context of the present invention is a compound having a LUMO≤−2.50 eV. Preferably, the LUMO is ≤−2.60 eV, more preferably ≤−2.65 eV, most preferably ≤−2.70 eV. The LUMO is the lowest unoccupied molecular orbital. The value of the LUMO of the compound is determined by quantum-chemical calculation, as described in general terms in the examples section at the back.

A hole-transporting compound in the context of the present invention is a compound having a HOMO≥−5.5 eV. The HOMO is preferably ≥−5.4 eV, more preferably ≥−5.3 eV. The HOMO is the highest occupied molecular orbital. The value of the HOMO of the compound is determined by quantum-chemical calculation, as described in general terms in the examples section at the back.

A bipolar compound in the context of the present invention is a compound which is both hole- and electron-transporting.

Suitable electron-conducting matrix compounds are selected from the substance classes of the triazines, the pyrimidines, the lactams, the metal complexes, especially the Be, Zn and Al complexes, the aromatic ketones, the aromatic phosphine oxides, the azaphospholes, the azaboroles substituted by at least one electron-conducting substituent, and the quinoxalines.

In a preferred embodiment of the invention, the electron-conducting compound is a purely organic compound, i.e. a compound containing no metals.

There follows a detailed description of the electronic device.

The electronic device according to the invention is preferably selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic dye-sensitised solar cells, organic optical detectors, organic photoreceptors, organic field-quench devices (O-FQDs), light-emitting electrochemical cells (LECs), organic laser diodes (O-lasers) and “organic plasmon emitting devices” (D. M. Koller et al., Nature Photonics 2008, 1-4), preferably organic electroluminescent devices (OLEDs).

The organic electroluminescent device comprises a cathode, an anode and at least one organic layer, preferably one emitting layer. Apart from these layers, it may also comprise further layers, for example in each case one or more hole-injection layers, hole-transport layers, hole-blocking layers, electron-transport layers, electron-injection layers, exciton-blocking layers, electron-blocking layers and/or charge-generation layers. It is likewise possible for interlayers, which have, for example, an exciton-blocking function, to be introduced between two emitting layers. However, it should be pointed out that each of these layers does not necessarily have to be present. The organic electroluminescent device here may comprise one emitting layer or a plurality of emitting layers. If a plurality of emission layers are present, these preferably have in total a plurality of emission maxima between 380 nm and 750 nm, resulting overall in white emission, i.e. various emitting compounds which are able to fluoresce or phosphoresce are used in the emitting layers. Particular preference is given to systems having three emitting layers, where the three layers exhibit blue, green and orange or red emission (for the basic structure see, for example, WO 2005/011013). These can be fluorescent or phosphorescent emission layers or hybrid systems, in which fluorescent and phosphorescent emission layers are combined with one another.

In the further layers of the inventive organic electroluminescent device, especially in the hole injection and transport layers and in the electron injection and transport layers, it is possible to use any materials as typically used according to the prior art. The hole transport layers may also be p-doped or the electron transport layers may also be n-doped. A p-doped layer is understood to mean a layer in which free holes are generated and which has increased conductivity as a result. A comprehensive discussion of doped transport layers in OLEDs can be found in Chem. Rev. 2007, 107, 1233. More preferably, the p-dopant is capable of oxidizing the hole transport material in the hole transport layer, i.e. has a sufficiently high redox potential, especially a higher redox potential than the hole transport material. Suitable dopants are in principle any compounds which are electron acceptor compounds and which can increase the conductivity of the organic layer by oxidizing the host. The person skilled in the art, in the context of his common knowledge in the art, is able to identify suitable compounds without any great effort. Especially suitable dopants are the compounds disclosed in WO 2011/073149, EP 1968131, EP 2276085, EP 2213662, EP 1722602, EP 2045848, DE 102007031220, U.S. Pat. Nos. 8,044,390, 8,057,712, WO 2009/003455, WO 2010/094378, WO 2011/120709 and US 2010/0096600.

The person skilled in the art will therefore be able, without exercising inventive skill, to use all the materials known for organic electroluminescent devices in combination with the emitting layer of the invention.

Preferred cathodes are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally, suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag, in which case combinations of the metals such as Ca/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. On the other hand, metal/metal oxide electrons (e.g. Al/Ni/NiOₓ, Al/PtOₓ) may also be preferred. In this case, at least one of the electrodes has to be transparent or semitransparent in order to enable the emission of light. A preferred structure uses a transparent anode. Preferred anode materials here are conductive mixed metal oxides. Particular preference is given to indium tin oxide (ITO) or indium zinc oxide (IZO). Preference is further given to conductive doped organic materials, especially conductive doped polymers.

The device is correspondingly (according to the application) structured, contact-connected and finally hermetically sealed, since the lifetime of such devices is severely shortened in the presence of water and/or air.

Additionally, preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. It is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example, M. S. Arnold et al., Appl. Phys. Lett. 2008, 92, 053301).

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution. Since the fluorescent compound having high steric shielding typically has good solubility in a multitude of standard organic solvents by virtue of the shielding groups, the production of the emitting layer from solution is preferred.

These methods are known in general terms to those skilled in the art and can be applied by those skilled in the art without exercising inventive skill to organic electroluminescent devices comprising the compounds of the invention.

The present invention therefore further provides a process for producing an inventive organic electroluminescent device, characterized in that at least one layer is applied by a sublimation method and/or in that at least one layer is applied by an OVPD (organic vapor phase deposition) method or with the aid of a carrier gas sublimation and/or in that at least one layer is applied from solution, by spin-coating or by a printing method.

A second object of the invention relates to compounds of the formula (III) or (IV),

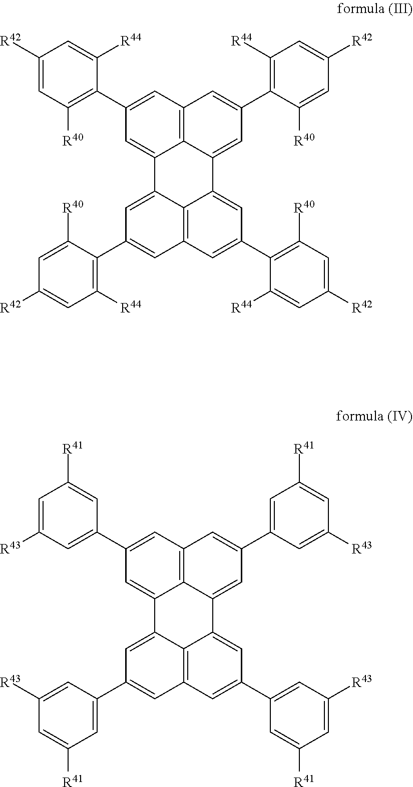

wherein
R⁴⁰, R⁴¹, R⁴², R⁴³ and R⁴⁴ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 24 aromatic ring atoms, which may in each case be substituted by one or more radicals R³²; and where
R³² is at each occurrence, identically or differently, selected from a straight-chain alkyl group having 1 to 10 carbon atoms, or a branched or cyclic alkyl group having 3 to 10 carbon atoms, or an aromatic ring system having 6 to 24 aromatic ring atoms.

In accordance with a preferred embodiment, in the compounds of formulae (III), the radicals R⁴⁰, R⁴², R⁴⁴ are defined as follows:
R⁴² is at each occurrence, identically or differently, selected from H, a straight-chain alkyl group having 1 to 10 carbon atoms, or a branched alkyl group having 3 to 10 carbon atoms;
R⁴⁰, R⁴⁴ are at each occurrence, identically or differently, selected from an aromatic ring system having 6 to 24 aromatic ring atoms, which may in each case be substituted by one or more radicals R³²; and
R³² is at each occurrence, identically or differently, selected from a straight-chain alkyl group having 1 to 6 carbon atoms, or a branched alkyl group having 3 to 6 carbon atoms.

In accordance with another preferred embodiment, in the compounds of formula (III), the radicals R⁴⁰, R⁴², R⁴⁴ are defined as follows:
R⁴⁰, R⁴², R⁴⁴ are at each occurrence, identically or differently, selected from an aromatic ring system having 6 to 24 aromatic ring atoms, which may in each case be substituted by one or more radicals R³²; and
R³² is at each occurrence, identically or differently, selected from a straight-chain alkyl group having 1 to 6 carbon atoms, or a branched alkyl group having 3 to 6 carbon atoms.

In accordance with a very preferred embodiment, the compounds of formula (III) are selected from the compounds of formulae (IIId), (IIIe) and (IIIf),

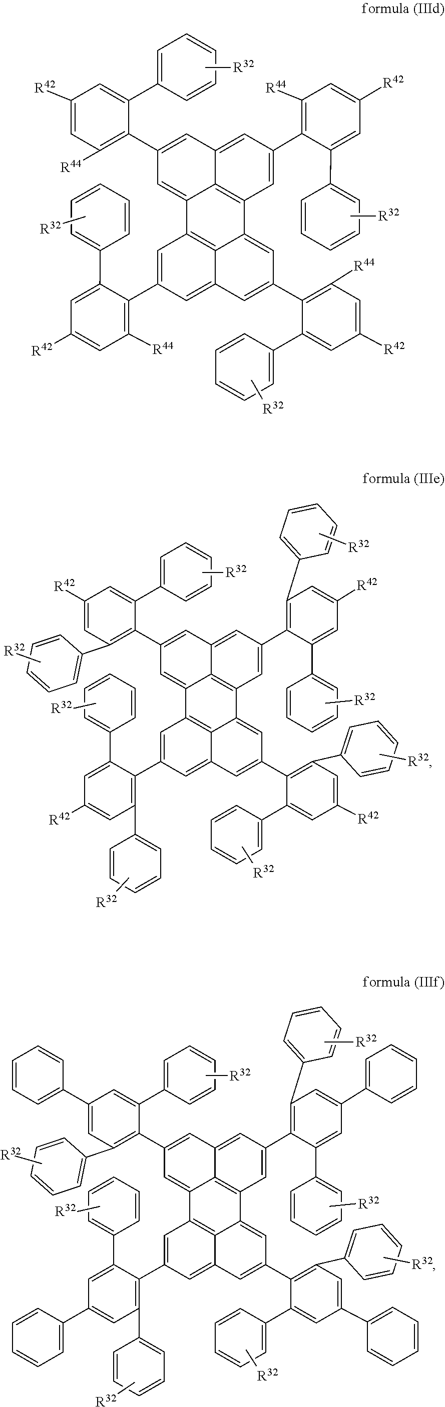

wherein
in each of formulae (IIId), (IIIe) and (IIIf) the phenyl groups indicated with —R³² are unsubstituted or substituted with one or more radicals R³²;
R⁴² and R⁴⁴ are at each occurrence, identically or differently, selected from H, a straight-chain alkyl group having 1 to 10 carbon atoms, or a branched alkyl group having 3 to 10 carbon atoms, where the above-mentioned groups may each be substituted by one or more radicals R³²; and
R³² is at each occurrence, identically or differently, selected from a straight-chain alkyl group having 1 to 6 carbon atoms, or a branched alkyl group having 3 to 6 carbon atoms.

In accordance with another preferred embodiment, in the compounds of formula (III), the radicals R⁴⁰, R⁴², R⁴⁴ are defined as follows:
R⁴² is at each occurrence, identically or differently, selected from H, a straight-chain alkyl group having 1 to 10 carbon atoms, or a branched alkyl group having 3 to 10 carbon atoms, or an aromatic ring system having 6 to 24 aromatic ring atoms, which may in each case be substituted by one or more radicals R³²;
R⁴⁰, R⁴⁴ are at each occurrence, identically or differently, selected from a straight-chain alkyl group having 1 to 10 carbon atoms, or a branched alkyl group having 3 to 10 carbon atoms, which may in each case be substituted by one or more radicals R³²; and
R³² is at each occurrence, identically or differently, selected from a straight-chain alkyl group having 1 to 6 carbon atoms, or a branched alkyl group having 3 to 6 carbon atoms.

In accordance with another very preferred embodiment, in the compounds of formula (III), the radicals R⁴⁰, R⁴², R⁴⁴ are defined as follows:
R⁴² is at each occurrence identically selected from H, a straight-chain alkyl group having 1 to 10 carbon atoms, or a branched alkyl group having 3 to 10 carbon atoms,
R⁴⁰, R⁴⁴ are at each occurrence identically selected from a straight-chain alkyl group having 1 to 10 carbon atoms, or a branched alkyl group having 3 to 10 carbon atoms.

The following compounds are examples of compounds of formulae (III) and (IV):

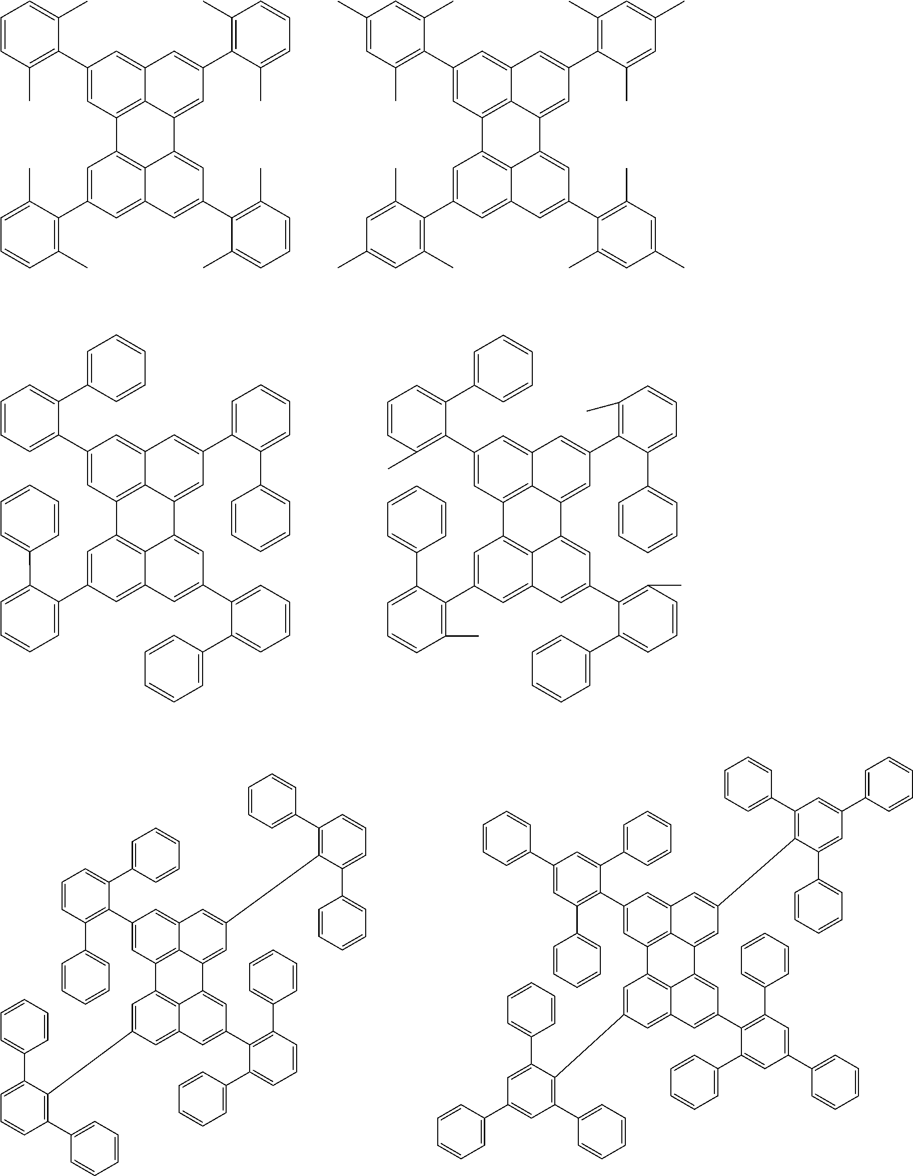

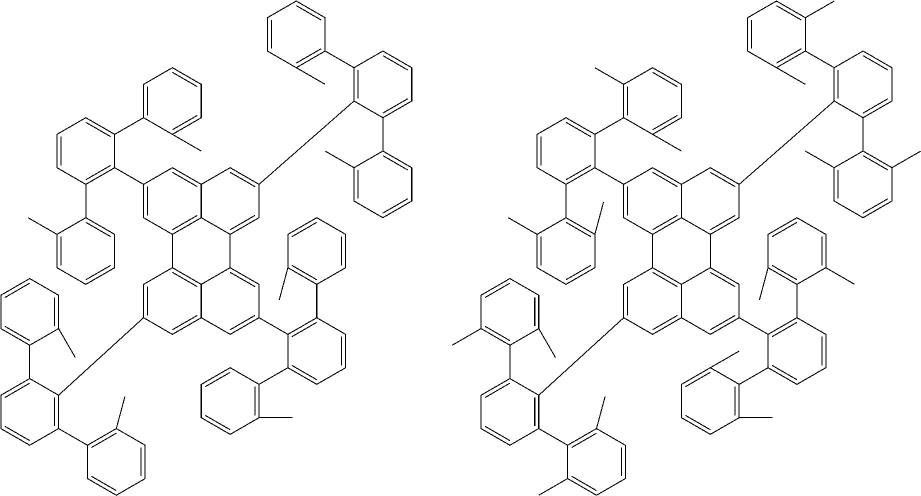

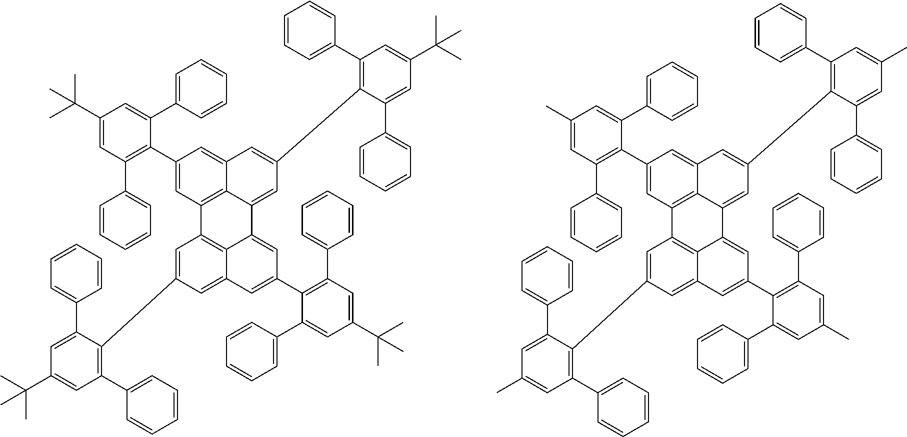

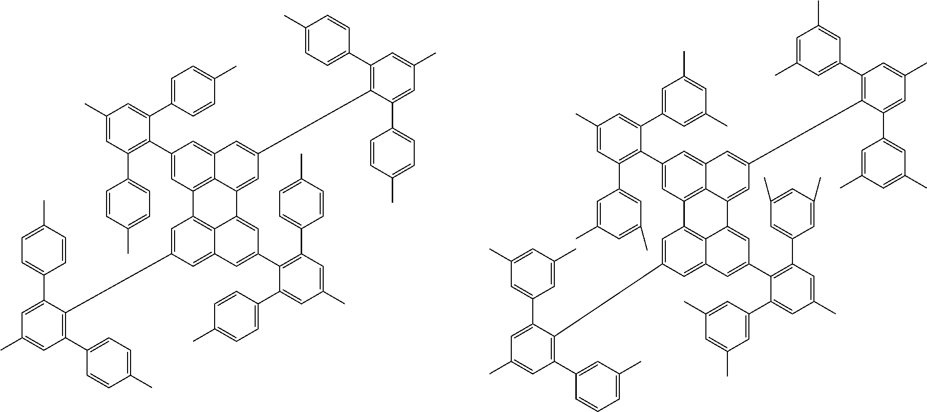

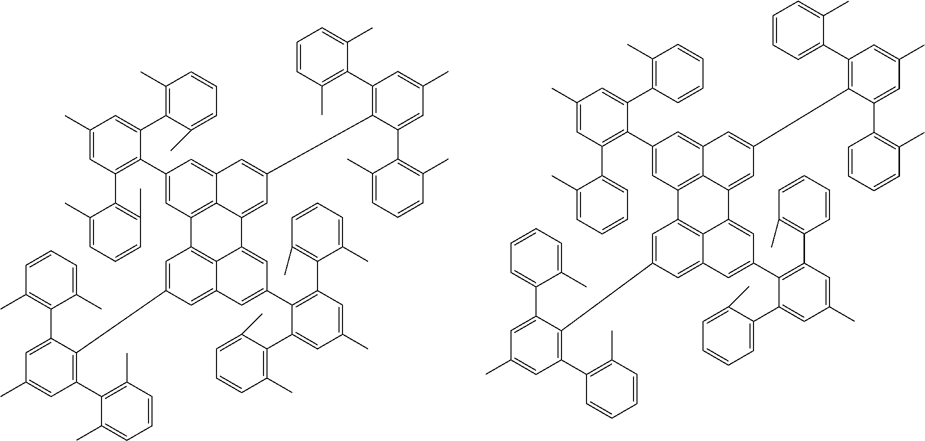

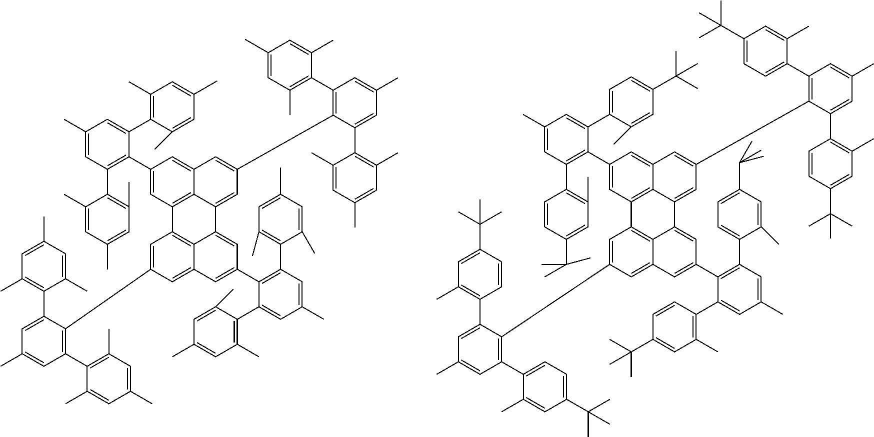

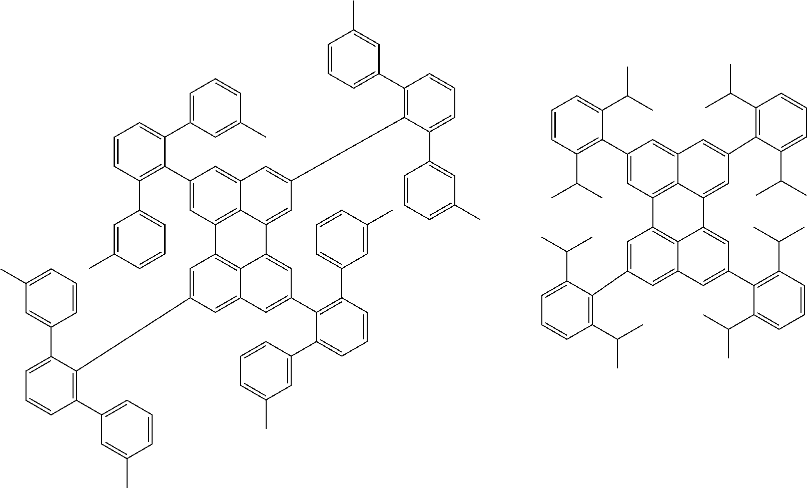

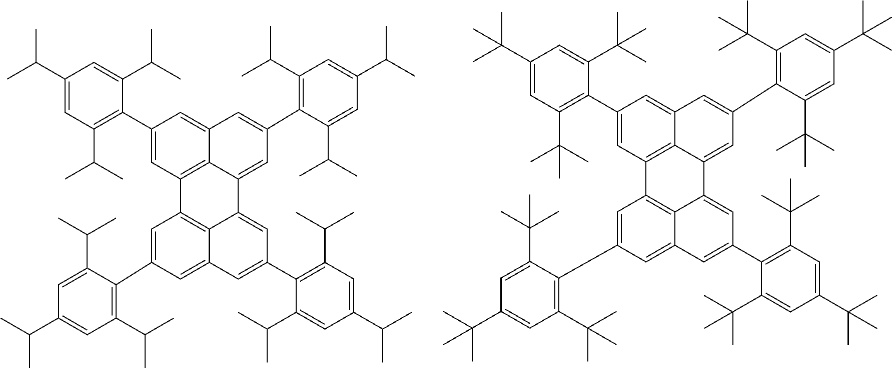

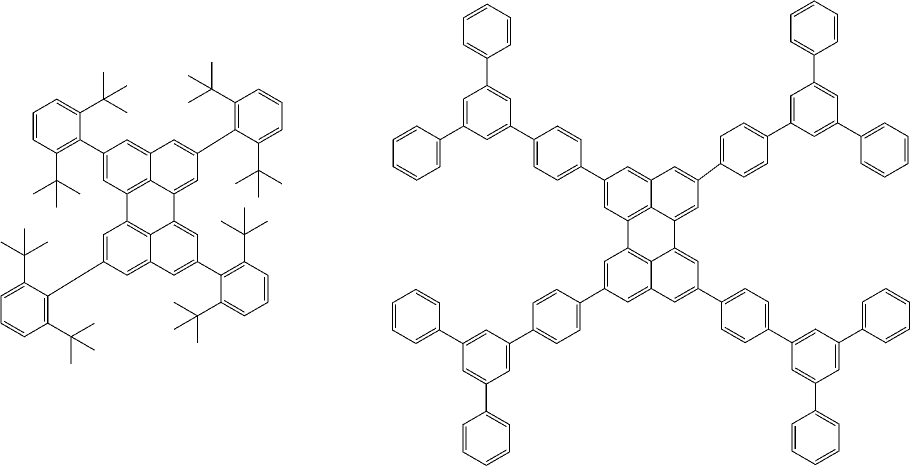

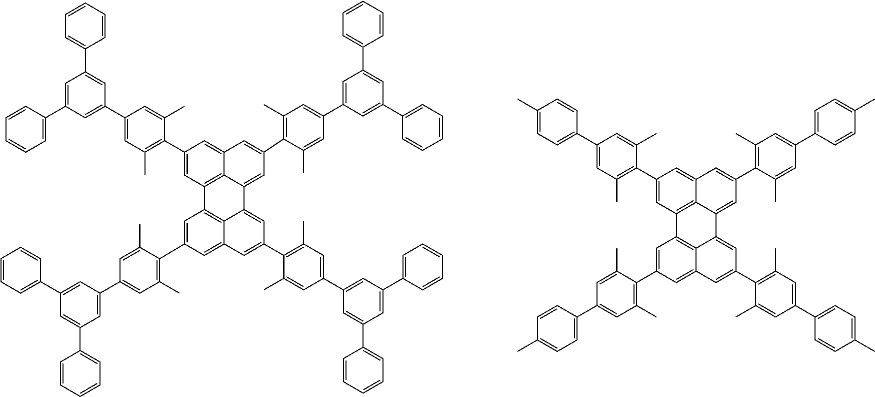

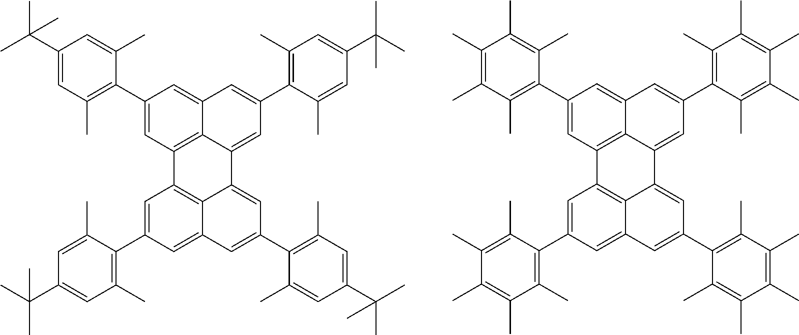

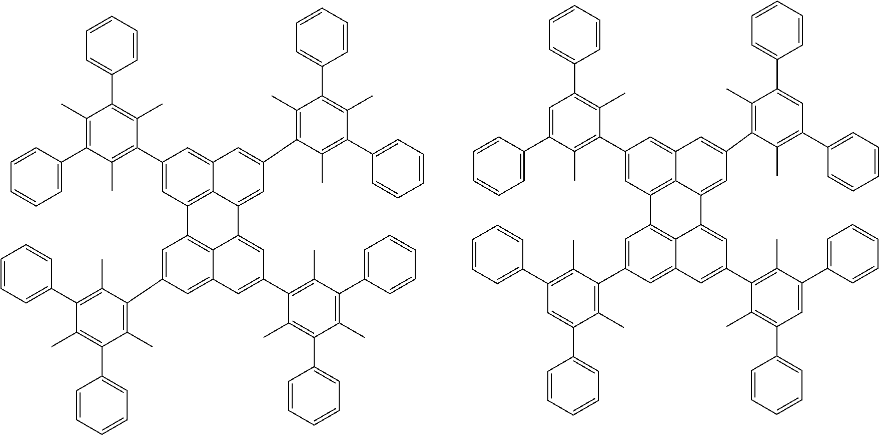

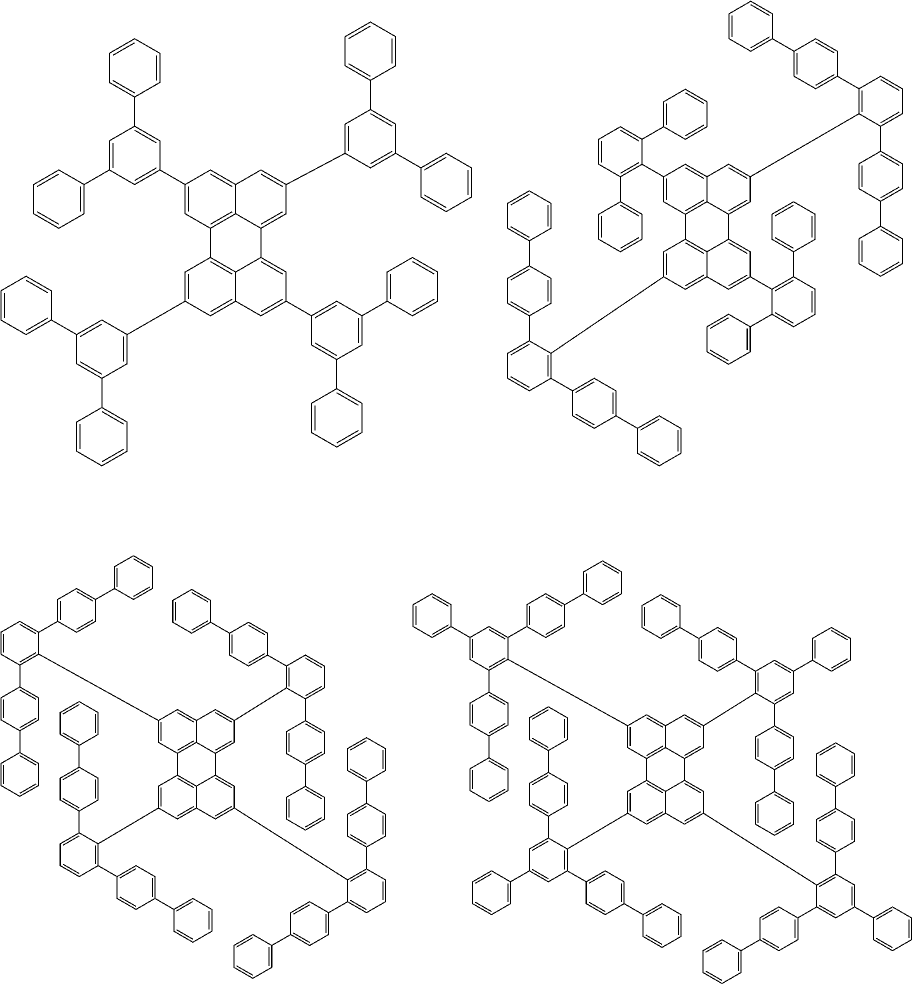

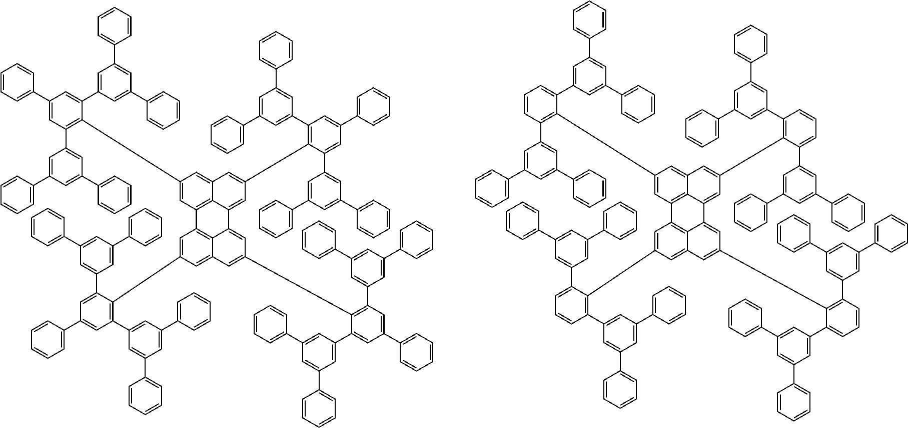

The compounds of formula (III) according to the invention can be prepared by synthesis steps known to the person skilled in the art, such as, for example, bromination, Suzuki coupling, Ullmann coupling, Hartwig-Buchwald coupling, 35 etc. An example of a suitable synthesis process is depicted in general terms in Scheme 1 below.

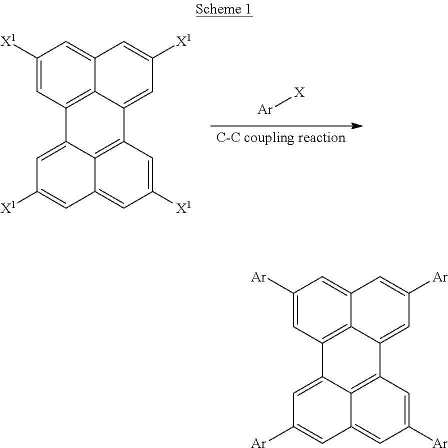

In Scheme 1, the symbols the X and X¹ represent a leaving group, preferably selected from a halogen (like Cl, Br, I), a boronic acid, a boronic ester or a triflate. The group Ar represents a substituted or unsubstituted aromatic ring system having 6 to 24 aromatic ring atoms, which may be substituted or unsubstituted.

The present invention therefore relates to a process for the synthesis of the compounds of the formula (III), comprising the following step a):
a) an organometallic coupling under Suzuki conditions between the 1-C, 5-C, 8-C and 11-C atoms of the perylene basic skeleton and a starting material Ar—X is carried out, where Ar is a substituted or unsubstituted aromatic group having 6 to 24 aromatic ring atoms and X is any desired suitable leaving group, preferably selected from a halide, a boronic acid, a boronic ester, a tosylate or a triflate.

The compounds of formulae (III) and (IV) may be combined with at least one organic functional material. Therefore, the present invention furthermore relates to a composition comprising a compound of formula (III) or (IV) and at least one organic or inorganic functional material selected from the group consisting of HTM, HIM, HBM, p-dopant, ETM, EIM, EBM, n-dopant, fluorescent emitter, phosphorescent emitter, delayed fluorescent material, matrix material, host material, wide band gap material, quantum material (preferably quantum dot).

For the processing of the compounds according to the invention from the liquid phase, for example by spin coating or by printing processes, formulations of the compounds according to the invention are necessary. These formulations can be, for example, solutions, dispersions or emulsions. It may be preferred to use mixtures of two or more solvents for this purpose. Suitable and preferred solvents are, for example, toluene, anisole, o-, m- or p-xylene, methyl benzoate, mesitylene, tetralin, veratrol, THF, methyl-THF, THP, chloro-benzene, dioxane, phenoxytoluene, in particular 3-phenoxytoluene, (−)-fenchone, 1,2,3,5-tetramethylbenzene, 1,2,4,5-tetramethylbenzene, 1-methyl-naphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclo-hexanol, cyclohexanone, cyclohexylbenzene, decalin, dodecylbenzene, ethyl benzoate, indane, methyl benzoate, NMP, p-cymene, phenetole, 1,4-di-isopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol-dimethyl ether, tetraethylene glycol dimethyl ether, 2-isopropylnaphthalene, pentylbenzene, hexylbenzene, heptylbenzene, octylbenzene, 1,1-bis(3,4-di-methylphenyl)ethane or mixtures of these solvents.

The present invention therefore furthermore relates to a formulation comprising a compound of formula (III) or (IV) and at least one further compound. The further compound may be, for example, a solvent, in particular one of the above-mentioned solvents or a mixture of these solvents. However, the further compound may also be at least one further organic or inorganic compound which is likewise employed in the electronic device, in particular one organic or inorganic functional material selected from the group consisting of HTM, HIM, HBM, p-dopant, ETM, EIM, EBM, n-dopant, fluorescent emitter, phosphorescent emitter, delayed fluorescent material, matrix material, host material, wide band gap material, quantum material (preferably quantum dot).

Suitable organic or inorganic functional materials, which can be used in a composition or formulation comprising a compound of formula (III) or (IV=) are indicated below in connection with the organic electroluminescent device. This further compound may also be polymeric.

The compounds of formulae (III) and (IV) and mixtures comprising these compounds are suitable for use in an electronic device. An electronic device here is taken to mean a device which comprises at least one layer which comprises at least one organic compound. However, the component here may also comprise inorganic materials or also layers built up entirely from inorganic materials.

The present invention therefore furthermore relates to the use of the com-pounds of formulae (III) and (IV) or mixtures comprising these compounds in an electronic device, in particular in an organic electroluminescent device.

The electronic device is preferably selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic dye-sensitised solar cells, organic optical detectors, organic photoreceptors, organic field-quench devices (O-FQDs), light-emitting electrochemical cells (LECs), organic laser diodes (O-lasers) and “organic plasmon emitting devices” (D. M. Koller et al., Nature Photonics 2008, 1-4), preferably organic electroluminescent devices (OLEDs, PLEDs), in particular phosphorescent OLEDs.

The organic electroluminescent device comprises a cathode, an anode and at least one emitting layer. Apart from these layers, it may also comprise further layers as described above.

The compounds of formulae (III) and (IV) according to the invention in accordance with the embodiments indicated above can be employed in various layers, depending on the precise structure and on the substitution. Preference is given to an organic electroluminescent device comprising a compound of the formula (III)), (IV) or in accordance with the preferred embodiments, as fluorescent emitters, emitters showing TADF (Thermally Activated Delayed Fluorescence), matrix material for fluorescent emitters. Particularly preferred is an organic electroluminescent device comprising a compound of the formula (III), (IV) or in accordance with the preferred embodiments as fluorescent emitters, more particularly blue-emitting fluorescent compound.

The compounds of formulae (III) and (IV) can also be employed in an electron-transport layer and/or in an electron-blocking or exciton-blocking layer and/or in a hole-transport layer, depending on the precise substitution. The preferred embodiments indicated above also apply to the use of the materials in organic electronic devices.

The compound according to the invention is particularly suitable for use as fluorescent blue-emitting compound. The electronic device concerned may comprise a single emitting layer comprising the compound of formula (III) or (IV) or it may comprise two or more emitting layers. The further emitting layers here may comprise one or more compounds of formula (III) or (IV), or alternatively other compounds.

If the compound of formula (III) or (IV) is employed as a fluorescent emitting compound in an emitting layer, it is preferably employed in combination with a sensitizer selected from compounds that exhibit delayed fluorescence or a phosphorescent compound. Suitable sensitizers corresponding to compounds exhibiting delayed fluorescence or phosphorescent compounds are described in more detailed above. If the compound of formula (III) or (IV) is employed as a fluorescent emitting compound in an emitting layer in combination with a sensitizer as described above, a further compound selected from matrix materials as described above may be present in the emitting layer comprising the compound of formula (III) or (IV).

The proportion of the emitting compound in the mixture of the emitting layer is between 0.1 and 50.0%, preferably between 0.5 and 20.0%, particularly preferably between 1.0 and 10.0%. Correspondingly, the proportion of the matrix material or matrix materials is between 50.0 and 99.9%, preferably between 80.0 and 99.5%, particularly preferably between 90.0 and 99.0%.

The specifications of the proportions in % are, for the purposes of the present application, taken to mean % by vol. if the compounds are applied from the gas phase and % by weight if the compounds are applied from solution.

Besides the matrix materials described above, known matrix materials for use in combination with fluorescent emitting compounds are selected from the classes of the oligoarylenes (for example 2,2′,7,7′-tetraphenylspirobifluorene in accordance with EP 676461 or dinaphthylanthracene), in particular the oligo-arylenes containing condensed aromatic groups, the oligoarylenevinylenes (for example DPVBi or spiro-DPVBi in accordance with EP 676461), the polypodal metal complexes (for example in accordance with WO 2004/081017), the hole-conducting compounds (for example in accordance with WO 2004/058911), the electron-conducting compounds, in particular ketones, phosphine oxides, sulfoxides, etc. (for example in accordance with WO 2005/084081 and WO 2005/084082), the atropisomers (for example in accordance with WO 2006/048268), the boronic acid derivatives (for example in accordance with WO 2006/117052) or the benzanthracenes (for example in accordance with WO 2008/145239). Particularly preferred matrix materials are selected from the classes of the oligoarylenes, comprising naphthalene, anthracene, benz-anthracene and/or pyrene or atropisomers of these compounds, the oligo-arylenevinylenes, the ketones, the phosphine oxides and the sulfoxides. Very particularly preferred matrix materials are selected from the classes of the oligoarylenes, comprising anthracene, benzanthracene, benzophenanthrene and/or pyrene or atropisomers of these compounds. An oligoarylene in the sense of this invention is intended to be taken to mean a compound in which at least three aryl or arylene groups are bonded to one another.

Particularly suitable matrix materials for use in combination with the com-pounds of the formula (III) or (IV) in the emitting layer, besides the matrix materials described above, are depicted in the following table:

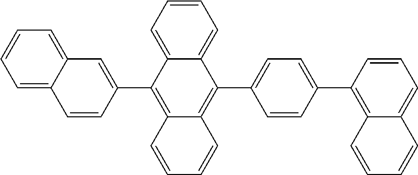 [SMILES: c1ccc2cc(-c3c4ccccc4c(-c4ccc(-c5cccc6ccccc56)cc4)c4ccccc34)ccc2c1]

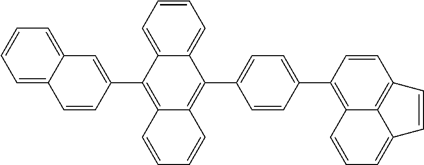 [SMILES: C1=Cc2ccc(-c3ccc(-c4c5ccccc5c(-c5ccc6ccccc6c5)c5ccccc45)cc3)c3cccc1c23]

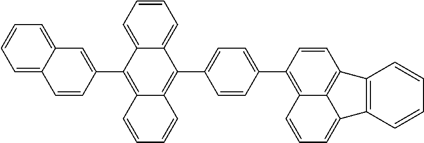 [SMILES: c1ccc2c(c1)-c1cccc3c(-c4ccc(-c5c6ccccc6c(-c6ccc7ccccc7c6)c6ccccc56)cc4)ccc-2c13]

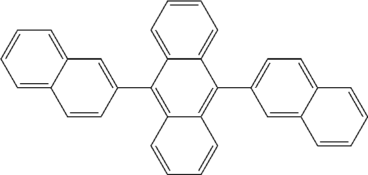 [SMILES: c1ccc2cc(-c3c4ccccc4c(-c4ccc5ccccc5c4)c4ccccc34)ccc2c1]

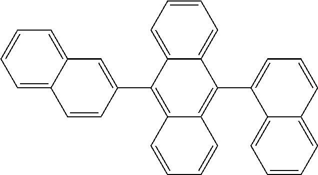 [SMILES: c1ccc2cc(-c3c4ccccc4c(-c4cccc5ccccc45)c4ccccc34)ccc2c1]

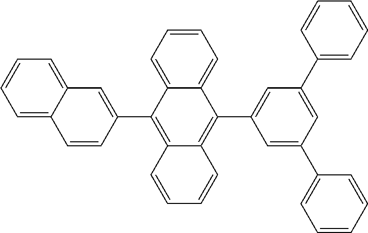 [SMILES: c1ccc(-c2cc(-c3ccccc3)cc(-c3c4ccccc4c(-c4ccc5ccccc5c4)c4ccccc34)c2)cc1]

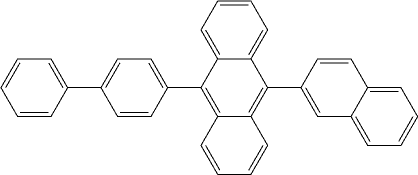 [SMILES: c1ccc(-c2ccc(-c3c4ccccc4c(-c4ccc5ccccc5c4)c4ccccc34)cc2)cc1]

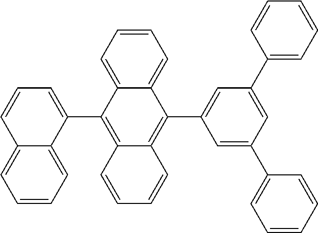 [SMILES: c1ccc(-c2cc(-c3ccccc3)cc(-c3c4ccccc4c(-c4cccc5ccccc45)c4ccccc34)c2)cc1]

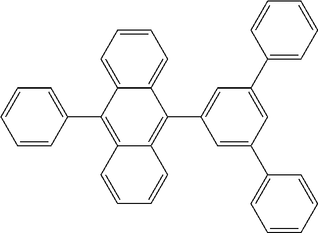 [SMILES: c1ccc(-c2cc(-c3ccccc3)cc(-c3c4ccccc4c(-c4ccccc4)c4ccccc34)c2)cc1]

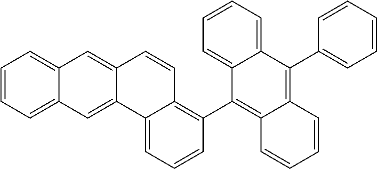 [SMILES: c1ccc(-c2c3ccccc3c(-c3cccc4c3ccc3cc5ccccc5cc34)c3ccccc23)cc1]

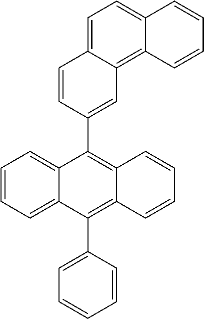 [SMILES: c1ccc(-c2c3ccccc3c(-c3ccc4ccc5ccccc5c4c3)c3ccccc23)cc1]

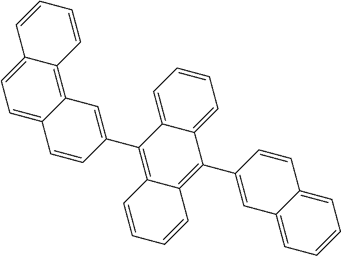 [SMILES: c1ccc2cc(-c3c4ccccc4c(-c4ccc5ccc6ccccc6c5c4)c4ccccc34)ccc2c1]

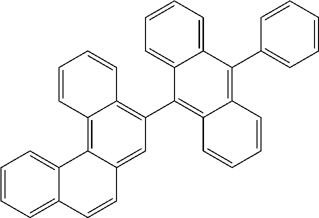 [SMILES: c1ccc(-c2c3ccccc3c(-c3cc4ccc5ccccc5c4c4ccccc34)c3ccccc23)cc1]

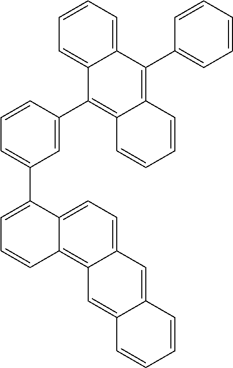 [SMILES: c1ccc(-c2c3ccccc3c(-c3cccc(-c4cccc5c4ccc4cc6ccccc6cc45)c3)c3ccccc23)cc1]

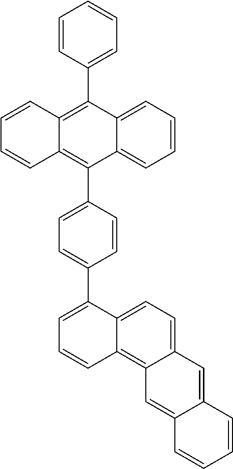 [SMILES: c1ccc(-c2c3ccccc3c(-c3ccc(-c4cccc5c4ccc4cc6ccccc6cc45)cc3)c3ccccc23)cc1]

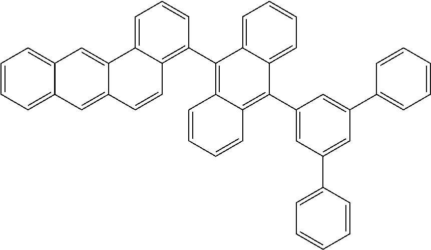 [SMILES: c1ccc(-c2cc(-c3ccccc3)cc(-c3c4ccccc4c(-c4cccc5c4ccc4cc6ccccc6cc45)c4ccccc34)c2)cc1]

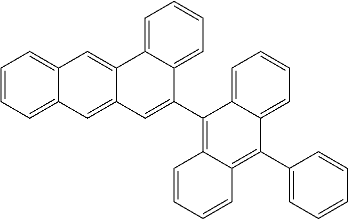 [SMILES: c1ccc(-c2c3ccccc3c(-c3cc4cc5ccccc5cc4c4ccccc34)c3ccccc23)cc1]

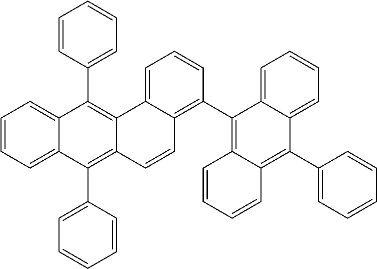 [SMILES: c1ccc(-c2c3ccccc3c(-c3cccc4c3ccc3c(-c5ccccc5)c5ccccc5c(-c5ccccc5)c34)c3ccccc23)cc1]

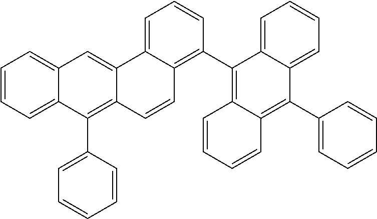 [SMILES: c1ccc(-c2c3ccccc3c(-c3cccc4c3ccc3c(-c5ccccc5)c5ccccc5cc34)c3ccccc23)cc1]

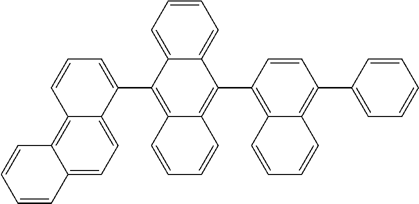 [SMILES: c1ccc(-c2ccc(-c3c4ccccc4c(-c4cccc5c4ccc4ccccc45)c4ccccc34)c3ccccc23)cc1]

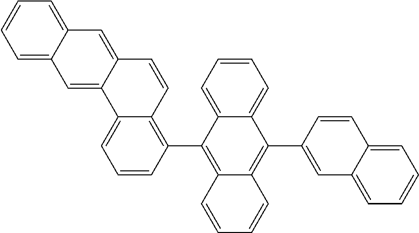 [SMILES: c1ccc2cc(-c3c4ccccc4c(-c4cccc5c4ccc4cc6ccccc6cc45)c4ccccc34)ccc2c1]

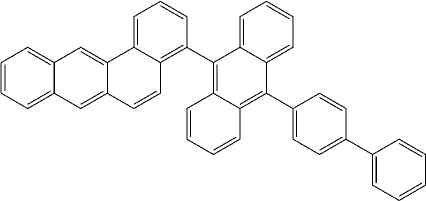 [SMILES: c1ccc(-c2ccc(-c3c4ccccc4c(-c4cccc5c4ccc4cc6ccccc6cc45)c4ccccc34)cc2)cc1]

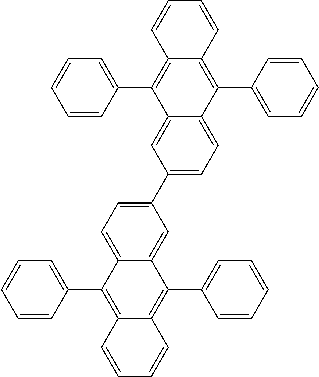 [SMILES: c1ccc(-c2c3ccccc3c(-c3ccccc3)c3cc(-c4ccc5c(-c6ccccc6)c6ccccc6c(-c6ccccc6)c5c4)ccc23)cc1]

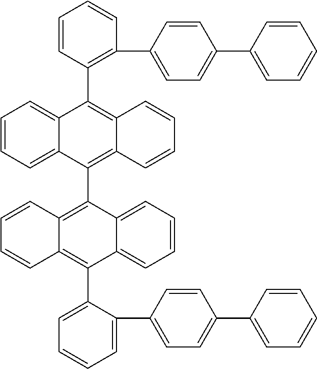 [SMILES: c1ccc(-c2ccc(-c3ccccc3-c3c4ccccc4c(-c4c5ccccc5c(-c5ccccc5-c5ccc(-c6ccccc6)cc5)c5ccccc45)c4ccccc34)cc2)cc1]

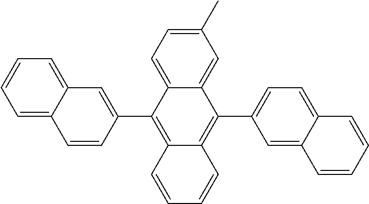 [SMILES: Cc1ccc2c(-c3ccc4ccccc4c3)c3ccccc3c(-c3ccc4ccccc4c3)c2c1]

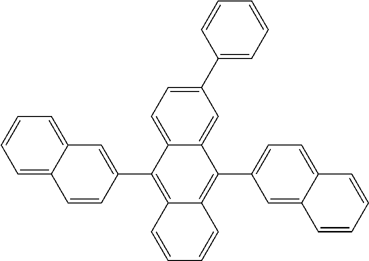 [SMILES: c1ccc(-c2ccc3c(-c4ccc5ccccc5c4)c4ccccc4c(-c4ccc5ccccc5c4)c3c2)cc1]

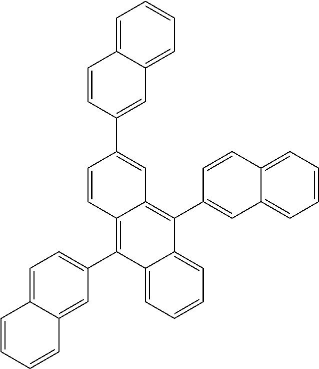 [SMILES: c1ccc2cc(-c3ccc4c(-c5ccc6ccccc6c5)c5ccccc5c(-c5ccc6ccccc6c5)c4c3)ccc2c1]

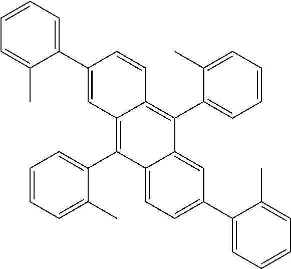 [SMILES: Cc1ccccc1-c1ccc2c(-c3ccccc3C)c3cc(-c4ccccc4C)ccc3c(-c3ccccc3C)c2c1]

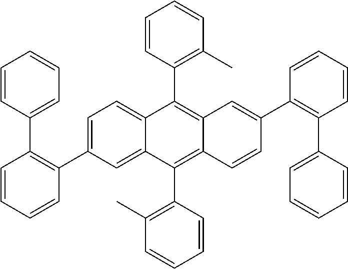 [SMILES: Cc1ccccc1-c1c2ccc(-c3ccccc3-c3ccccc3)cc2c(-c2ccccc2C)c2ccc(-c3ccccc3-c3ccccc3)cc12]

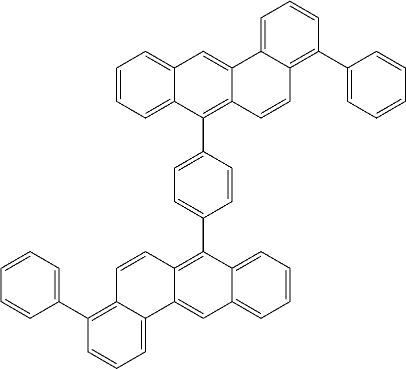 [SMILES: c1ccc(-c2cccc3c2ccc2c(-c4ccc(-c5c6ccccc6cc6c5ccc5c(-c7ccccc7)cccc56)cc4)c4ccccc4cc23)cc1]

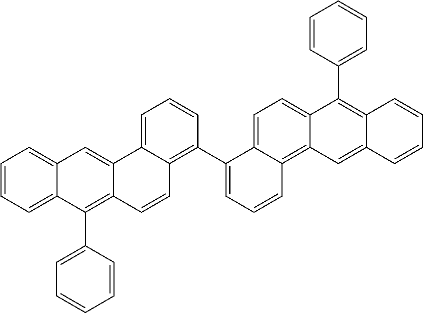 [SMILES: c1ccc(-c2c3ccccc3cc3c2ccc2c(-c4cccc5c4ccc4c(-c6ccccc6)c6ccccc6cc45)cccc23)cc1]

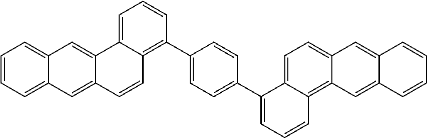 [SMILES: c1ccc2cc3c(ccc4c(-c5ccc(-c6cccc7c6ccc6cc8ccccc8cc67)cc5)cccc43)cc2c1]

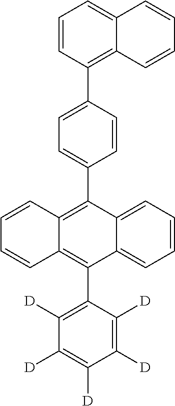 [SMILES: [2H]c1c([2H])c([2H])c(-c2c3ccccc3c(-c3ccc(-c4cccc5ccccc45)cc3)c3ccccc23)c([2H])c1[2H]]

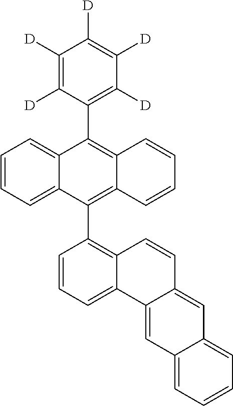 [SMILES: [2H]c1c([2H])c([2H])c(-c2c3ccccc3c(-c3cccc4c3ccc3cc5ccccc5cc34)c3ccccc23)c([2H])c1[2H]]

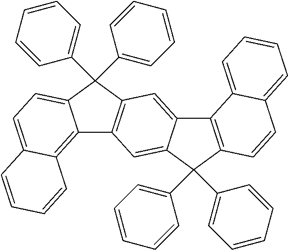 [SMILES: c1ccc(C2(c3ccccc3)c3cc4c(cc3-c3c2ccc2ccccc32)C(c2ccccc2)(c2ccccc2)c2ccc3ccccc3c2-4)cc1]

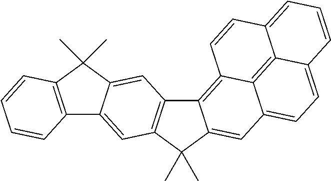 [SMILES: CC1(C)c2ccccc2-c2cc3c(cc21)-c1c(cc2ccc4cccc5ccc1c2c45)C3(C)C]

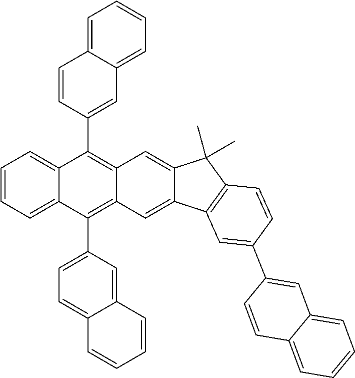 [SMILES: CC1(C)c2ccc(-c3ccc4ccccc4c3)cc2-c2cc3c(-c4ccc5ccccc5c4)c4ccccc4c(-c4ccc5ccccc5c4)c3cc21]

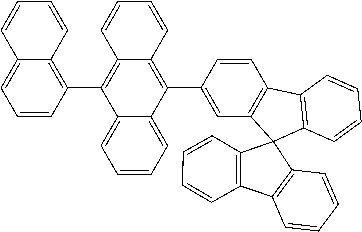 [SMILES: c1ccc2c(c1)-c1ccccc1C21c2ccccc2-c2ccc(-c3c4ccccc4c(-c4cccc5ccccc45)c4ccccc34)cc21]

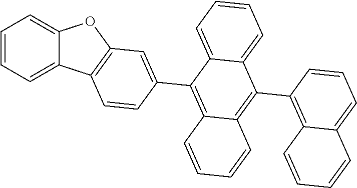 [SMILES: c1ccc2c(-c3c4ccccc4c(-c4ccc5c(c4)oc4ccccc45)c4ccccc34)cccc2c1]

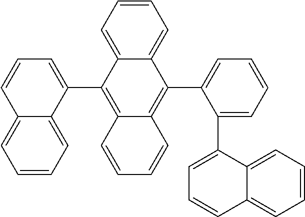 [SMILES: c1ccc(-c2c3ccccc3c(-c3cccc4ccccc34)c3ccccc23)c(-c2cccc3ccccc23)c1]

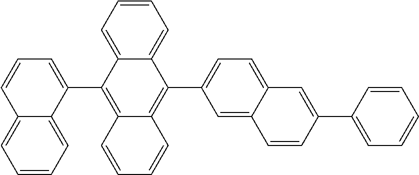 [SMILES: c1ccc(-c2ccc3cc(-c4c5ccccc5c(-c5cccc6ccccc56)c5ccccc45)ccc3c2)cc1]

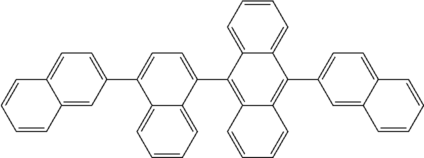 [SMILES: c1ccc2cc(-c3ccc(-c4c5ccccc5c(-c5ccc6ccccc6c5)c5ccccc45)c4ccccc34)ccc2c1]

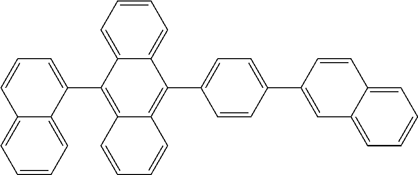 [SMILES: c1ccc2cc(-c3ccc(-c4c5ccccc5c(-c5cccc6ccccc56)c5ccccc45)cc3)ccc2c1]

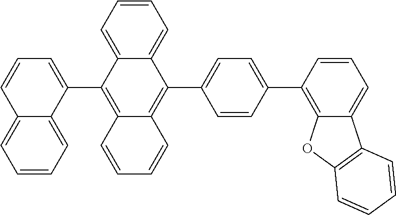 [SMILES: c1ccc2c(-c3c4ccccc4c(-c4ccc(-c5cccc6c5oc5ccccc56)cc4)c4ccccc34)cccc2c1]

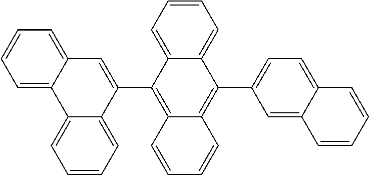 [SMILES: c1ccc2cc(-c3c4ccccc4c(-c4cc5ccccc5c5ccccc45)c4ccccc34)ccc2c1]

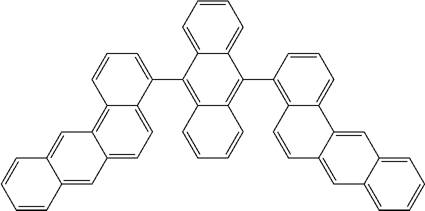 [SMILES: c1ccc2cc3c(ccc4c(-c5c6ccccc6c(-c6cccc7c6ccc6cc8ccccc8cc67)c6ccccc56)cccc43)cc2c1]

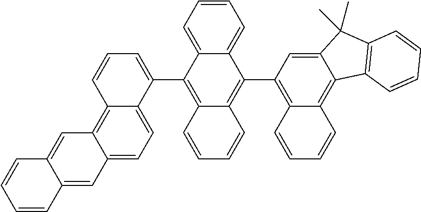 [SMILES: CC1(C)c2ccccc2-c2c1cc(-c1c3ccccc3c(-c3cccc4c3ccc3cc5ccccc5cc34)c3ccccc13)c1ccccc21]

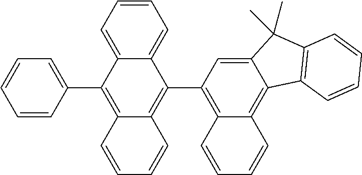 [SMILES: CC1(C)c2ccccc2-c2c1cc(-c1c3ccccc3c(-c3ccccc3)c3ccccc13)c1ccccc21]

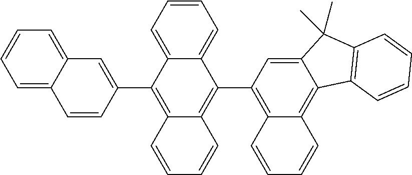 [SMILES: CC1(C)c2ccccc2-c2c1cc(-c1c3ccccc3c(-c3ccc4ccccc4c3)c3ccccc13)c1ccccc21]

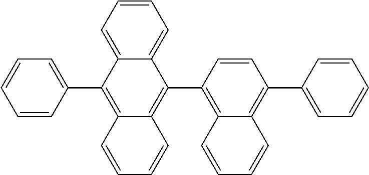 [SMILES: c1ccc(-c2ccc(-c3c4ccccc4c(-c4ccccc4)c4ccccc34)c3ccccc23)cc1]

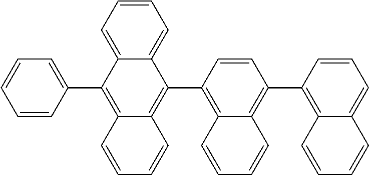 [SMILES: c1ccc(-c2c3ccccc3c(-c3ccc(-c4cccc5ccccc45)c4ccccc34)c3ccccc23)cc1]

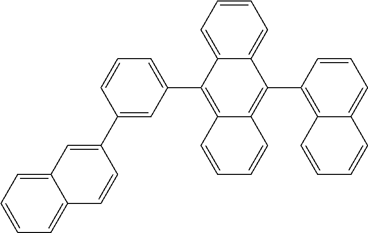 [SMILES: c1cc(-c2ccc3ccccc3c2)cc(-c2c3ccccc3c(-c3cccc4ccccc34)c3ccccc23)c1]

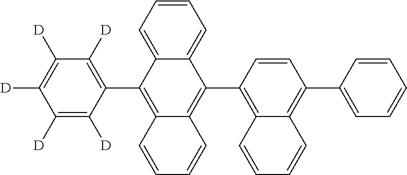 [SMILES: [2H]c1c([2H])c([2H])c(-c2c3ccccc3c(-c3ccc(-c4ccccc4)c4ccccc34)c3ccccc23)c([2H])c1[2H]]

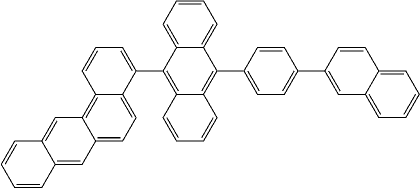 [SMILES: c1ccc2cc(-c3ccc(-c4c5ccccc5c(-c5cccc6c5ccc5cc7ccccc7cc56)c5ccccc45)cc3)ccc2c1]

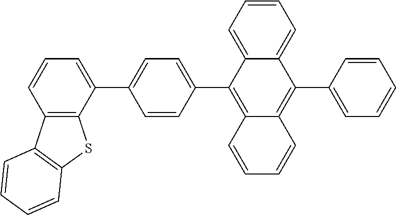 [SMILES: c1ccc(-c2c3ccccc3c(-c3ccc(-c4cccc5c4sc4ccccc45)cc3)c3ccccc23)cc1]

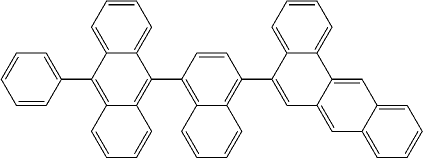 [SMILES: c1ccc(-c2c3ccccc3c(-c3ccc(-c4cc5cc6ccccc6cc5c5ccccc45)c4ccccc34)c3ccccc23)cc1]

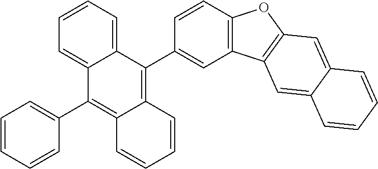 [SMILES: c1ccc(-c2c3ccccc3c(-c3ccc4oc5cc6ccccc6cc5c4c3)c3ccccc23)cc1]

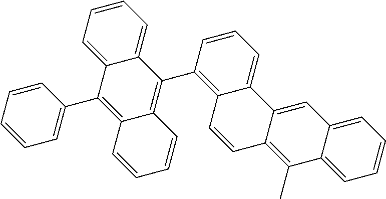 [SMILES: Cc1c2ccccc2cc2c1ccc1c(-c3c4ccccc4c(-c4ccccc4)c4ccccc34)cccc12]

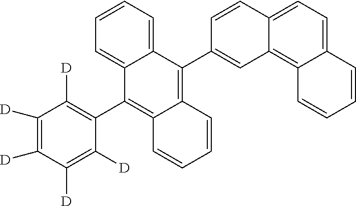 [SMILES: [2H]c1c([2H])c([2H])c(-c2c3ccccc3c(-c3ccc4ccc5ccccc5c4c3)c3ccccc23)c([2H])c1[2H]]

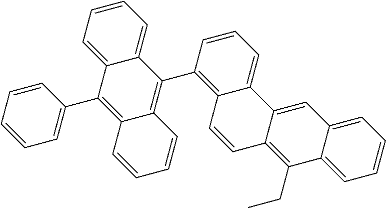 [SMILES: CCc1c2ccccc2cc2c1ccc1c(-c3c4ccccc4c(-c4ccccc4)c4ccccc34)cccc12]

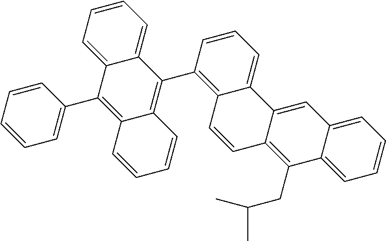 [SMILES: CC(C)Cc1c2ccccc2cc2c1ccc1c(-c3c4ccccc4c(-c4ccccc4)c4ccccc34)cccc12]

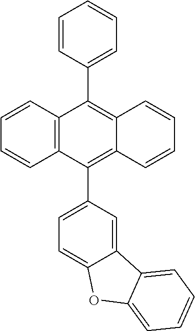 [SMILES: c1ccc(-c2c3ccccc3c(-c3ccc4oc5ccccc5c4c3)c3ccccc23)cc1]

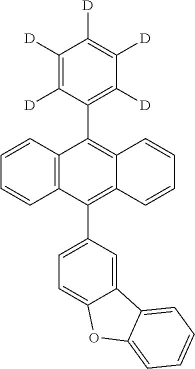 [SMILES: [2H]c1c([2H])c([2H])c(-c2c3ccccc3c(-c3ccc4oc5ccccc5c4c3)c3ccccc23)c([2H])c1[2H]]

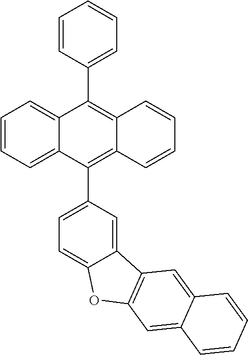 [SMILES: c1ccc(-c2c3ccccc3c(-c3ccc4oc5cc6ccccc6cc5c4c3)c3ccccc23)cc1]

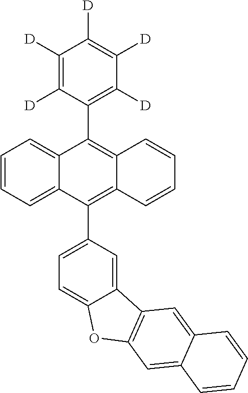 [SMILES: [2H]c1c([2H])c([2H])c(-c2c3ccccc3c(-c3ccc4oc5cc6ccccc6cc5c4c3)c3ccccc23)c([2H])c1[2H]]

If the compound of formula (III) or (IV) is employed as a fluorescent emitting compound in an emitting layer, it is preferably employed in combination with a sensitizer selected from compounds that exhibit delayed fluorescence or a phosphorescent compound. If the compound of formula (III) or (IV) is employed as a fluorescent emitting compound in an emitting layer, it may be employed in combination with one or more other fluorescent emitting compounds. Preferably, it may be employed in combination with one or more other sterically hindered fluorescent emitters as described in WO 2015/135624.

Other preferred fluorescent emitters, besides the compounds of formula (III) or (IV), are selected from the class of the arylamines. An arylamine in the sense of this invention is taken to mean a compound which contains three substituted or unsubstituted aromatic or heteroaromatic ring systems bonded directly to the nitrogen. At least one of these aromatic or heteroaromatic ring systems is preferably a condensed ring system, particularly preferably having at least 14 aromatic ring atoms. Preferred examples thereof are aromatic anthracenamines, aromatic anthracenediamines, aromatic pyrenamines, aromatic pyrenediamines, aromatic chrysenamines or aromatic chrysene-diamines. An aromatic anthracenamine is taken to mean a compound in which one diarylamino group is bonded directly to an anthracene group, preferably in the 9-position. An aromatic anthracenediamine is taken to mean a compound in which two diarylamino groups are bonded directly to an anthracene group, preferably in the 9,10-position. Aromatic pyrenamines, pyrenediamines, chrysenamines and chrysenediamines are defined analogously thereto, where the diarylamino groups are preferably bonded to the pyrene in the 1-position or in the 1,6-position. Further preferred emitters are indenofluorenamines or indenofluorenediamines, for example in accordance with WO 2006/108497 or WO 2006/122630, benzoindenofluorenamines or benzoindenofluorene-diamines, for example in accordance with WO 2008/006449, and dibenzo-indenofluorenamines or dibenzoindenofluorenediamines, for example in accordance with WO 2007/140847, and the indenofluorene derivatives containing condensed aryl groups which are disclosed in WO 2010/012328. Still further preferred emitters are benzanthracene derivatives as disclosed in WO 2015/158409, anthracene derivatives as disclosed in WO 2017/036573, fluorene dimers like in WO 2016/150544 or phenoxazine derivatives as disclosed in WO 2017/028940 and WO 2017/028941. Preference is likewise given to the pyrenarylamines disclosed in WO 2012/048780 and WO 2013/185871. Preference is likewise given to the benzoindenofluorenamines disclosed in WO 2014/037077, the benzofluorenamines disclosed in WO 2014/106522, the indenofluorenes disclosed in WO 2014/111269 or WO 2017/036574 and the sterically hindered fluorescent emitters as described in WO 2015/135624.

Examples of preferred fluorescent emitting compounds, besides the compounds of formula (III) and (IV), which can be used in combination with the compounds of formulae (III) and (IV) in an emitting layer or which can be used in another emitting layer of the same device are depicted in the following table:

| | |
|---|---|
| 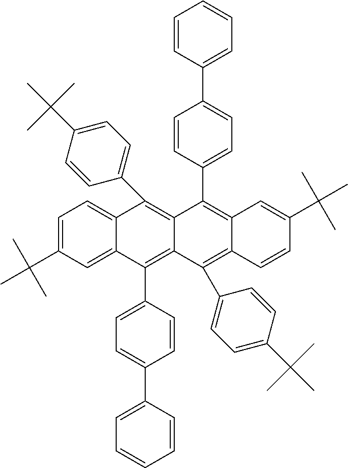 [SMILES: CC(C)(C)c1ccc(-c2c3ccc(C(C)(C)C)cc3c(-c3ccc(-c4ccccc4)cc3)c3c(-c4ccc(C(C)(C)C)cc4)c4ccc(C(C)(C)C)cc4c(-c4ccc(-c5ccccc5)cc4)c23)cc1] | 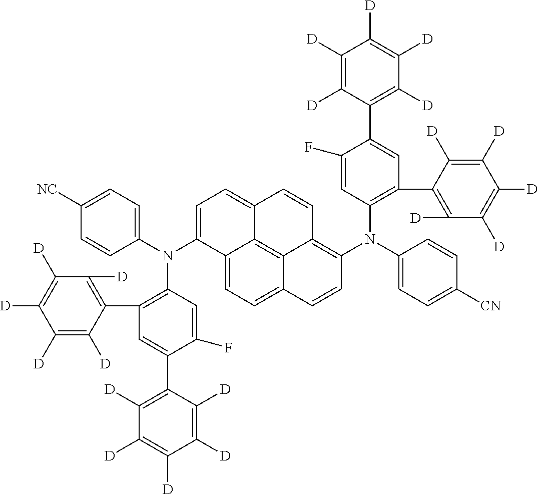 [SMILES: [2H]c1c([2H])c([2H])c(-c2cc(-c3c([2H])c([2H])c([2H])c([2H])c3[2H])c(N(c3ccc(C#N)cc3)c3ccc4ccc5c(N(c6ccc(C#N)cc6)c6cc(F)c(-c7c([2H])c([2H])c([2H])c([2H])c7[2H])cc6-c6c([2H])c([2H])c([2H])c([2H])c6[2H])ccc6ccc3c4c65)cc2F)c([2H])c1[2H]] |
| 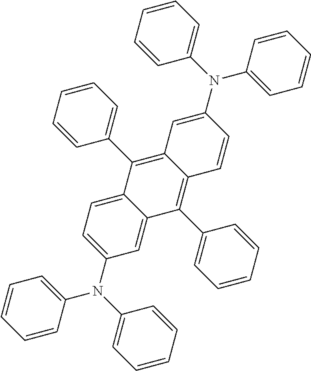 [SMILES: c1ccc(-c2c3ccc(N(c4ccccc4)c4ccccc4)cc3c(-c3ccccc3)c3ccc(N(c4ccccc4)c4ccccc4)cc23)cc1] | 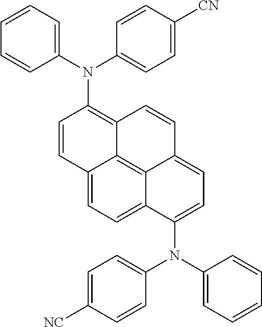 [SMILES: N#Cc1ccc(N(c2ccccc2)c2ccc3ccc4c(N(c5ccccc5)c5ccc(C#N)cc5)ccc5ccc2c3c54)cc1] |
| 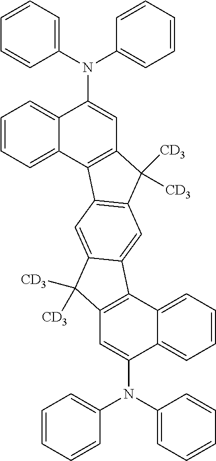 [SMILES: [2H]C([2H])([2H])C1(C([2H])([2H])[2H])c2cc3c(cc2-c2c1cc(N(c1ccccc1)c1ccccc1)c1ccccc21)C(C([2H])([2H])[2H])(C([2H])([2H])[2H])c1cc(N(c2ccccc2)c2ccccc2)c2ccccc2c1-3] | 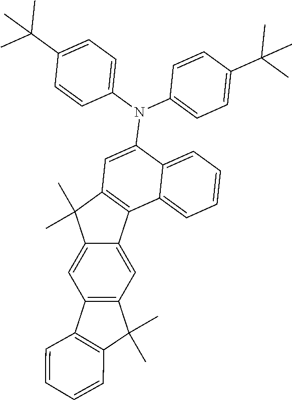 [SMILES: CC(C)(C)c1ccc(N(c2ccc(C(C)(C)C)cc2)c2cc3c(c4ccccc24)-c2cc4c(cc2C3(C)C)-c2ccccc2C4(C)C)cc1] |
| 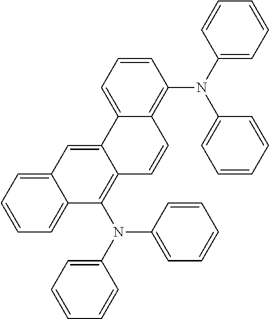 [SMILES: c1ccc(N(c2ccccc2)c2cccc3c2ccc2c(N(c4ccccc4)c4ccccc4)c4ccccc4cc23)cc1] | 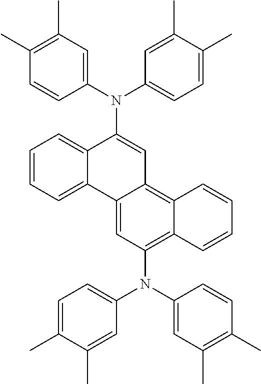 |
| 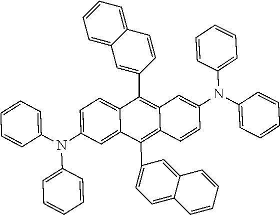 [SMILES: c1ccc(N(c2ccccc2)c2ccc3c(-c4ccc5ccccc5c4)c4cc(N(c5ccccc5)c5ccccc5)ccc4c(-c4ccc5ccccc5c4)c3c2)cc1] | 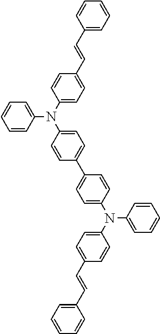 [SMILES: C(=C\c1ccc(N(c2ccccc2)c2ccc(-c3ccc(N(c4ccccc4)c4ccc(/C=C/c5ccccc5)cc4)cc3)cc2)cc1)/c1ccccc1] |
| 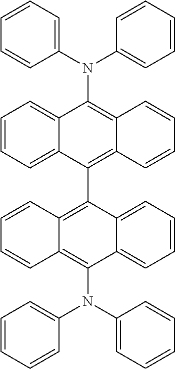 [SMILES: c1ccc(N(c2ccccc2)c2c3ccccc3c(-c3c4ccccc4c(N(c4ccccc4)c4ccccc4)c4ccccc34)c3ccccc23)cc1] | 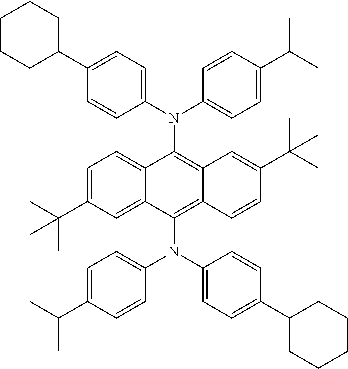 [SMILES: CC(C)c1ccc(N(c2ccc(C3CCCCC3)cc2)c2c3ccc(C(C)(C)C)cc3c(N(c3ccc(C(C)C)cc3)c3ccc(C4CCCCC4)cc3)c3ccc(C(C)(C)C)cc23)cc1] |
| 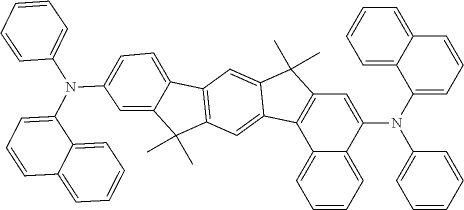 [SMILES: CC1(C)c2cc(N(c3ccccc3)c3cccc4ccccc34)ccc2-c2cc3c(cc21)-c1c(cc(N(c2ccccc2)c2cccc4ccccc24)c2ccccc12)C3(C)C] | 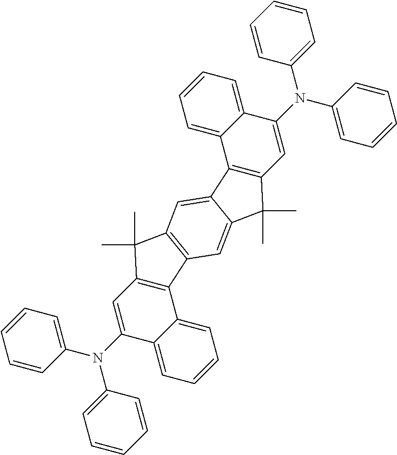 [SMILES: CC1(C)c2cc3c(cc2-c2c1cc(N(c1ccccc1)c1ccccc1)c1ccccc21)C(C)(C)c1cc(N(c2ccccc2)c2ccccc2)c2ccccc2c1-3] |
| 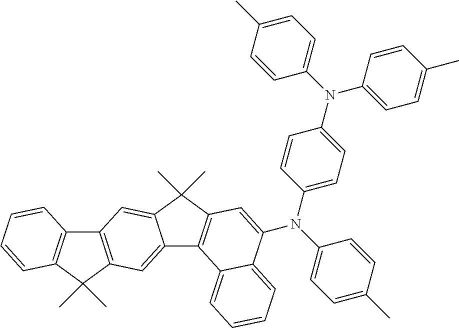 [SMILES: Cc1ccc(N(c2ccc(C)cc2)c2ccc(N(c3ccc(C)cc3)c3cc4c(c5ccccc35)-c3cc5c(cc3C4(C)C)-c3ccccc3C5(C)C)cc2)cc1] | 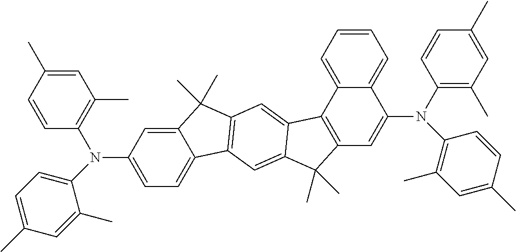 |
| 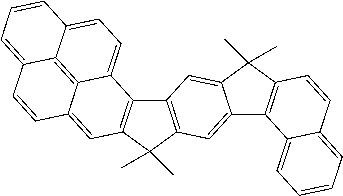 [SMILES: CC1(C)c2cc3c(cc2-c2c1ccc1ccccc21)C(C)(C)c1cc2ccc4cccc5ccc(c1-3)c2c45] | 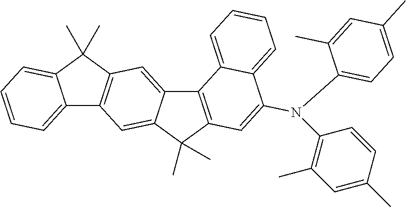 [SMILES: Cc1ccc(N(c2ccc(C)cc2C)c2cc3c(c4ccccc24)-c2cc4c(cc2C3(C)C)-c2ccccc2C4(C)C)c(C)c1] |
| 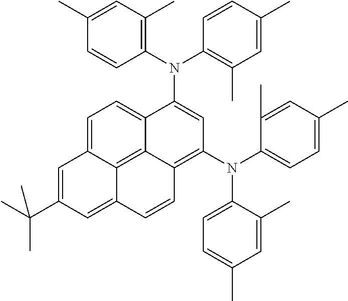 [SMILES: Cc1ccc(N(c2ccc(C)cc2C)c2cc(N(c3ccc(C)cc3C)c3ccc(C)cc3C)c3ccc4cc(C(C)(C)C)cc5ccc2c3c54)c(C)c1] | 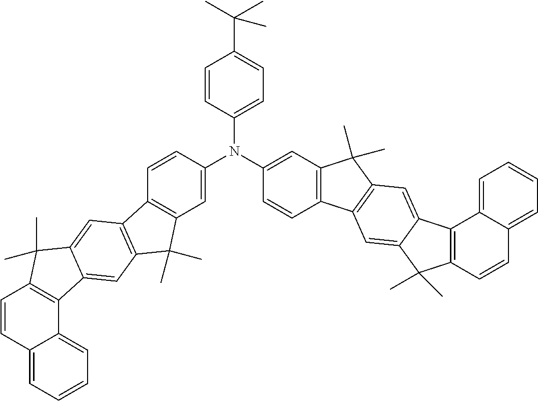 [SMILES: CC(C)(C)c1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1] |
| 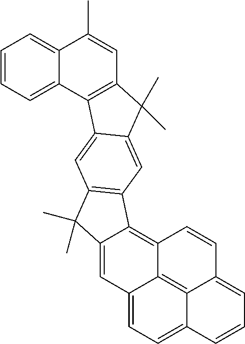 [SMILES: Cc1cc2c(c3ccccc13)-c1cc3c(cc1C2(C)C)-c1c(cc2ccc4cccc5ccc1c2c45)C3(C)C] | 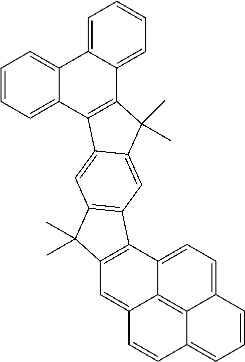 [SMILES: CC1(C)c2cc3c(cc2-c2c1cc1ccc4cccc5ccc2c1c45)C(C)(C)c1c-3c2ccccc2c2ccccc12] |
| 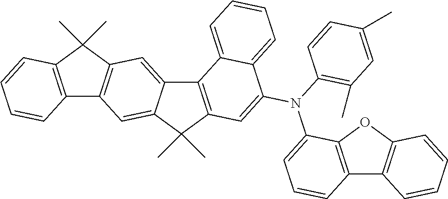 [SMILES: Cc1ccc(N(c2cc3c(c4ccccc24)-c2cc4c(cc2C3(C)C)-c2ccccc2C4(C)C)c2cccc3c2oc2ccccc23)c(C)c1] | 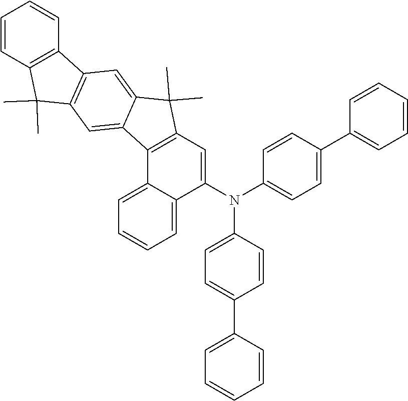 [SMILES: CC1(C)c2ccccc2-c2cc3c(cc21)-c1c(cc(N(c2ccc(-c4ccccc4)cc2)c2ccc(-c4ccccc4)cc2)c2ccccc12)C3(C)C] |
| 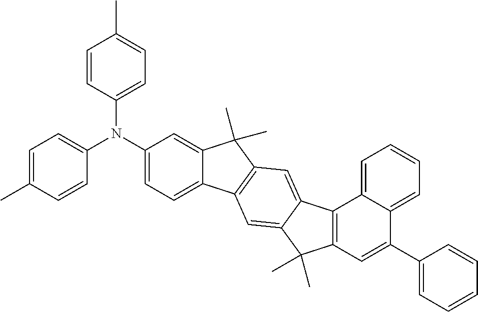 [SMILES: Cc1ccc(N(c2ccc(C)cc2)c2ccc3c(c2)C(C)(C)c2cc4c(cc2-3)C(C)(C)c2cc(-c3ccccc3)c3ccccc3c2-4)cc1] | 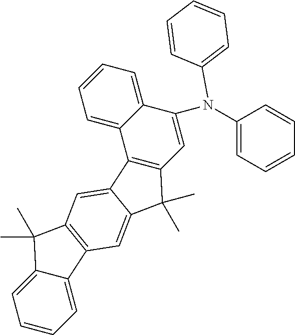 [SMILES: CC1(C)c2ccccc2-c2cc3c(cc21)-c1c(cc(N(c2ccccc2)c2ccccc2)c2ccccc12)C3(C)C] |
| 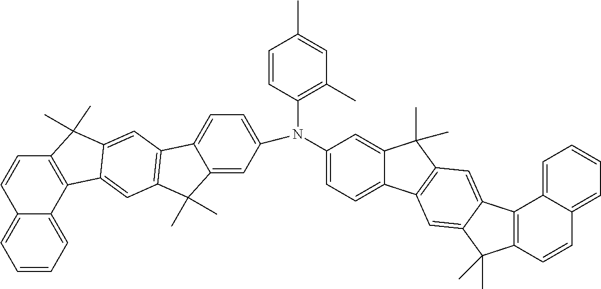 [SMILES: Cc1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)c(C)c1] | 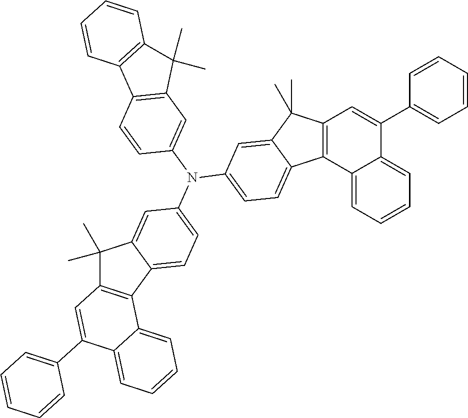 [SMILES: CC1(C)c2ccccc2-c2ccc(N(c3ccc4c(c3)C(C)(C)c3cc(-c5ccccc5)c5ccccc5c3-4)c3ccc4c(c3)C(C)(C)c3cc(-c5ccccc5)c5ccccc5c3-4)cc21] |
| 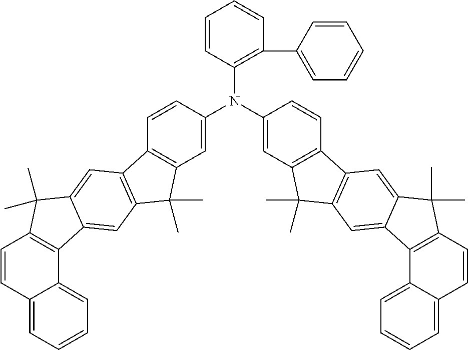 [SMILES: CC1(C)c2cc(N(c3ccc4c(c3)C(C)(C)c3cc5c(cc3-4)C(C)(C)c3ccc4ccccc4c3-5)c3ccccc3-c3ccccc3)ccc2-c2cc3c(cc21)-c1c(ccc2ccccc12)C3(C)C] | 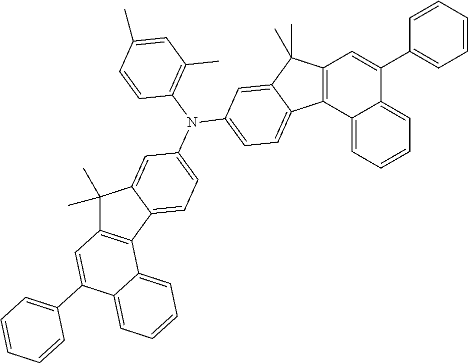 [SMILES: Cc1ccc(N(c2ccc3c(c2)C(C)(C)c2cc(-c4ccccc4)c4ccccc4c2-3)c2ccc3c(c2)C(C)(C)c2cc(-c4ccccc4)c4ccccc4c2-3)c(C)c1] |
| 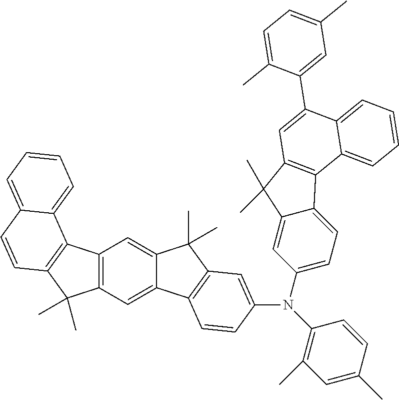 [SMILES: Cc1ccc(N(c2ccc3c(c2)C(C)(C)c2cc4c(cc2-3)C(C)(C)c2ccc3ccccc3c2-4)c2ccc3c(c2)C(C)(C)c2cc(-c4cc(C)ccc4C)c4ccccc4c2-3)c(C)c1] | 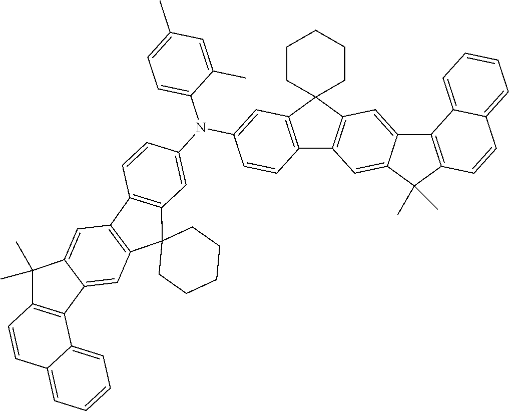 [SMILES: Cc1ccc(N(c2ccc3c(c2)C2(CCCCC2)c2cc4c(cc2-3)C(C)(C)c2ccc3ccccc3c2-4)c2ccc3c(c2)C2(CCCCC2)c2cc4c(cc2-3)C(C)(C)c2ccc3ccccc3c2-4)c(C)c1] |
| 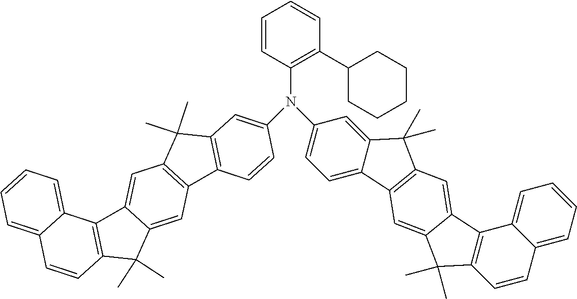 [SMILES: CC1(C)c2cc(N(c3ccc4c(c3)C(C)(C)c3cc5c(cc3-4)C(C)(C)c3ccc4ccccc4c3-5)c3ccccc3C3CCCCC3)ccc2-c2cc3c(cc21)-c1c(ccc2ccccc12)C3(C)C] | 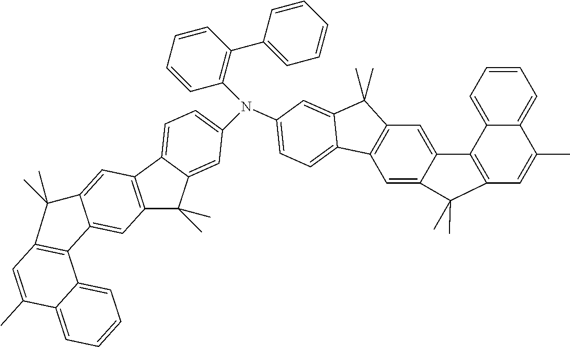 |
| 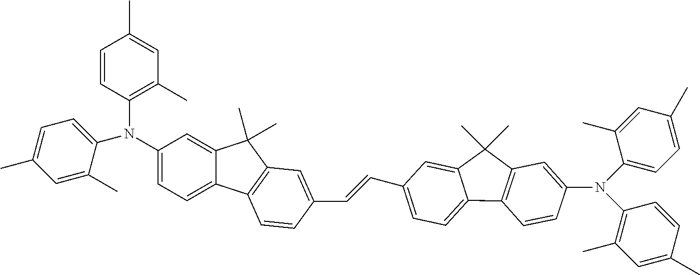 | 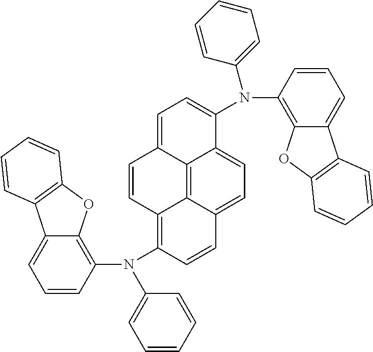 [SMILES: c1ccc(N(c2ccc3ccc4c(N(c5ccccc5)c5cccc6c5oc5ccccc56)ccc5ccc2c3c54)c2cccc3c2oc2ccccc23)cc1] |
| 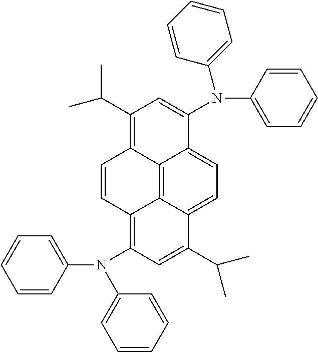 [SMILES: CC(C)c1cc(N(c2ccccc2)c2ccccc2)c2ccc3c(C(C)C)cc(N(c4ccccc4)c4ccccc4)c4ccc1c2c34] | 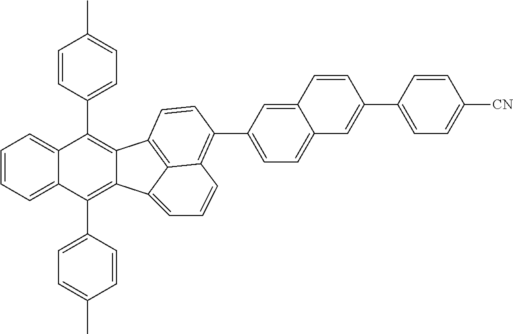 [SMILES: Cc1ccc(-c2c3c(c(-c4ccc(C)cc4)c4ccccc24)-c2ccc(-c4ccc5cc(-c6ccc(C#N)cc6)ccc5c4)c4cccc-3c24)cc1] |
| 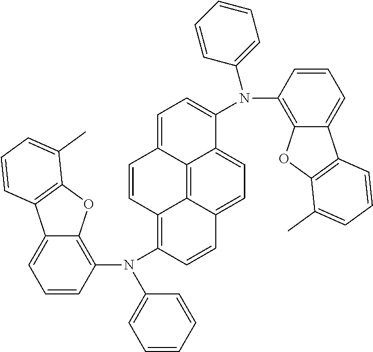 [SMILES: Cc1cccc2c1oc1c(N(c3ccccc3)c3ccc4ccc5c(N(c6ccccc6)c6cccc7c6oc6c(C)cccc67)ccc6ccc3c4c65)cccc12] | 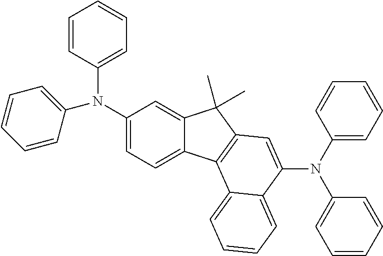 [SMILES: CC1(C)c2cc(N(c3ccccc3)c3ccccc3)ccc2-c2c1cc(N(c1ccccc1)c1ccccc1)c1ccccc21] |

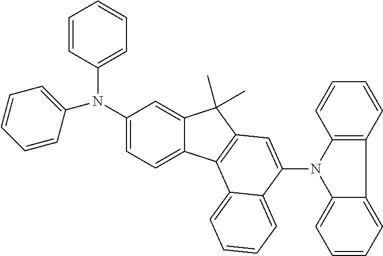 [SMILES: CC1(C)c2cc(N(c3ccccc3)c3ccccc3)ccc2-c2c1cc(-n1c3ccccc3c3ccccc31)c1ccccc21]

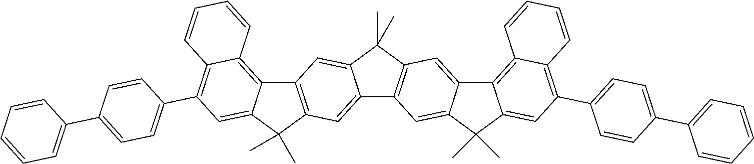 [SMILES: CC1(C)c2cc3c(cc2-c2cc4c(cc21)-c1c(cc(-c2ccc(-c5ccccc5)cc2)c2ccccc12)C4(C)C)C(C)(C)c1cc(-c2ccc(-c4ccccc4)cc2)c2ccccc2c1-3]

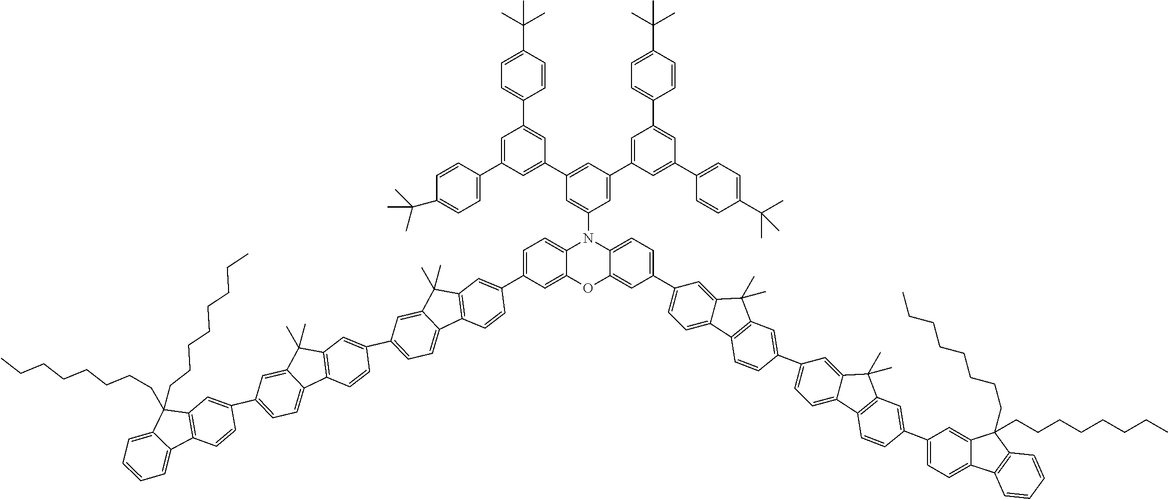 [SMILES: CCCCCCCCC1(CCCCCCCC)c2ccccc2-c2ccc(-c3ccc4c(c3)C(C)(C)c3cc(-c5ccc6c(c5)C(C)(C)c5cc(-c7ccc8c(c7)Oc7cc(-c9ccc%10c(c9)C(C)(C)c9cc(-c%11ccc%12c(c%11)C(C)(C)c%11cc(-c%13ccc%14c(c%13)C(CCCCCCCC)(CCCCCCCC)c%13ccccc%13-%14)ccc%11-%12)ccc9-%10)ccc7N8c7cc(-c8cc(-c9ccc(C(C)(C)C)cc9)cc(-c9ccc(C(C)(C)C)cc9)c8)cc(-c8cc(-c9ccc(C(C)(C)C)cc9)cc(-c9ccc(C(C)(C)C)cc9)c8)c7)ccc5-6)ccc3-4)cc21]

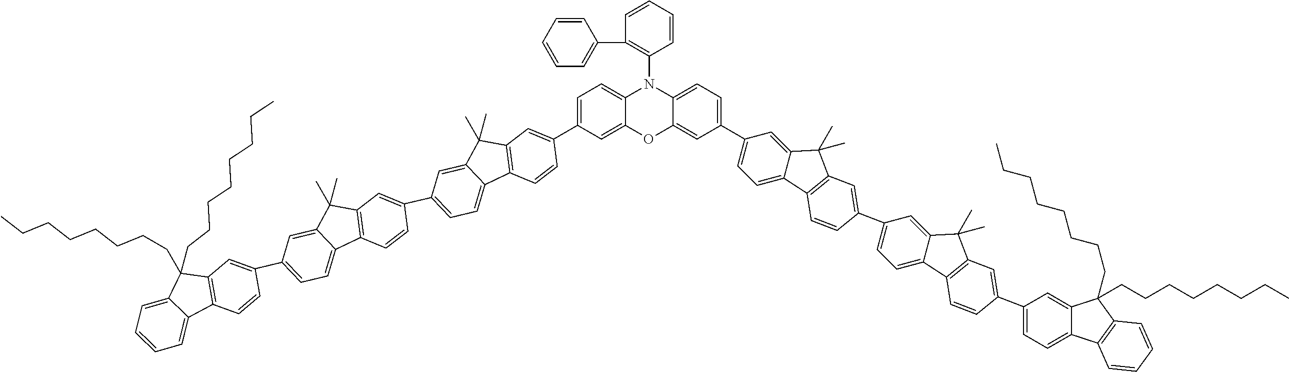 [SMILES: CCCCCCCCC1(CCCCCCCC)c2ccccc2-c2ccc(-c3ccc4c(c3)C(C)(C)c3cc(-c5ccc6c(c5)C(C)(C)c5cc(-c7ccc8c(c7)Oc7cc(-c9ccc%10c(c9)C(C)(C)c9cc(-c%11ccc%12c(c%11)C(C)(C)c%11cc(-c%13ccc%14c(c%13)C(CCCCCCCC)(CCCCCCCC)c%13ccccc%13-%14)ccc%11-%12)ccc9-%10)ccc7N8c7ccccc7-c7ccccc7)ccc5-6)ccc3-4)cc21]

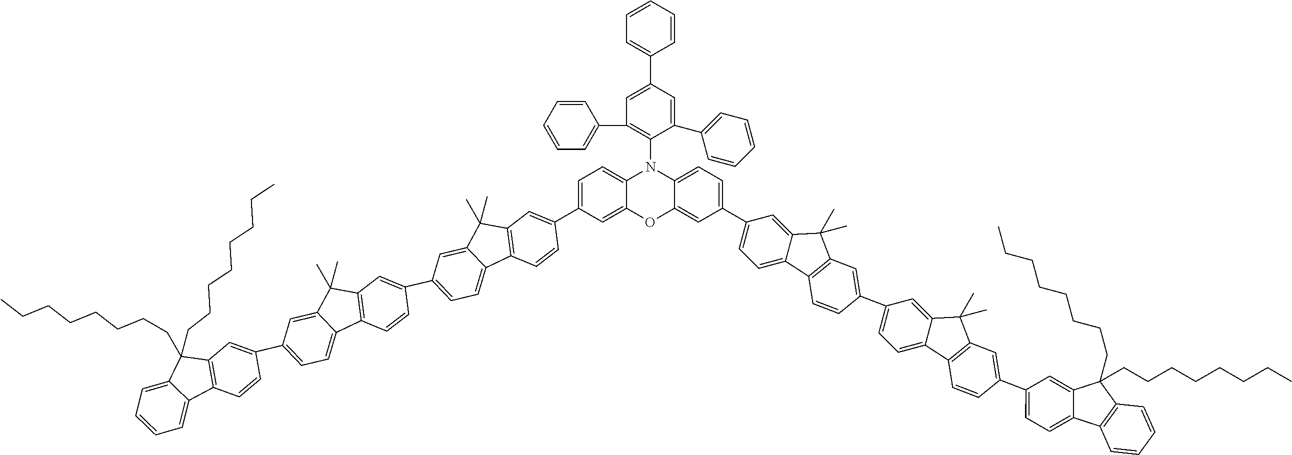 [SMILES: CCCCCCCCC1(CCCCCCCC)c2ccccc2-c2ccc(-c3ccc4c(c3)C(C)(C)c3cc(-c5ccc6c(c5)C(C)(C)c5cc(-c7ccc8c(c7)Oc7cc(-c9ccc%10c(c9)C(C)(C)c9cc(-c%11ccc%12c(c%11)C(C)(C)c%11cc(-c%13ccc%14c(c%13)C(CCCCCCCC)(CCCCCCCC)c%13ccccc%13-%14)ccc%11-%12)ccc9-%10)ccc7N8c7c(-c8ccccc8)cc(-c8ccccc8)cc7-c7ccccc7)ccc5-6)ccc3-4)cc21]

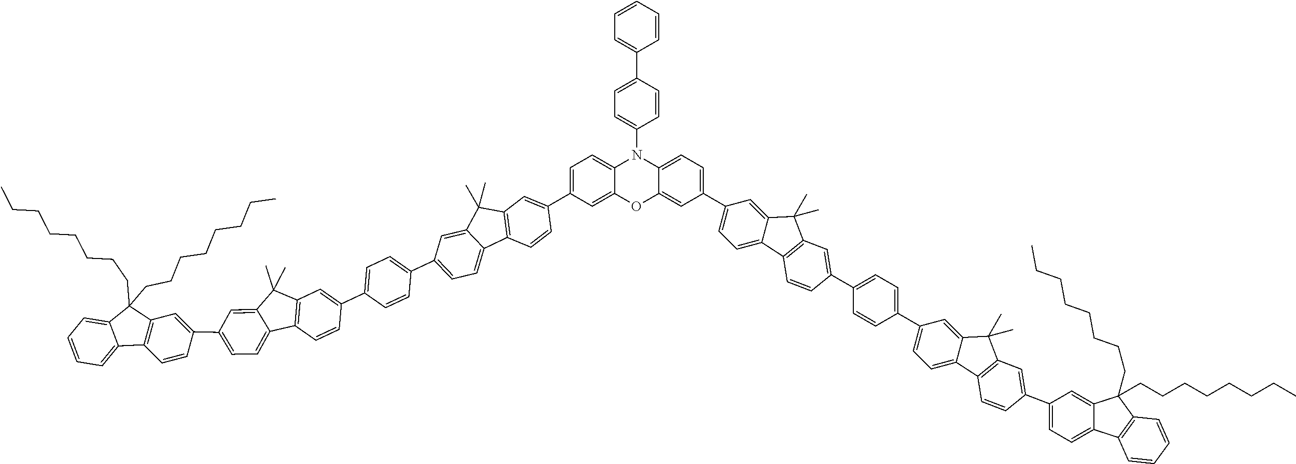 [SMILES: CCCCCCCCC1(CCCCCCCC)c2ccccc2-c2ccc(-c3ccc4c(c3)C(C)(C)c3cc(-c5ccc(-c6ccc7c(c6)C(C)(C)c6cc(-c8ccc9c(c8)Oc8cc(-c%10ccc%11c(c%10)C(C)(C)c%10cc(-c%12ccc(-c%13ccc%14c(c%13)C(C)(C)c%13cc(-c%15ccc%16c(c%15)C(CCCCCCCC)(CCCCCCCC)c%15ccccc%15-%16)ccc%13-%14)cc%12)ccc%10-%11)ccc8N9c8ccc(-c9ccccc9)cc8)ccc6-7)cc5)ccc3-4)cc21]

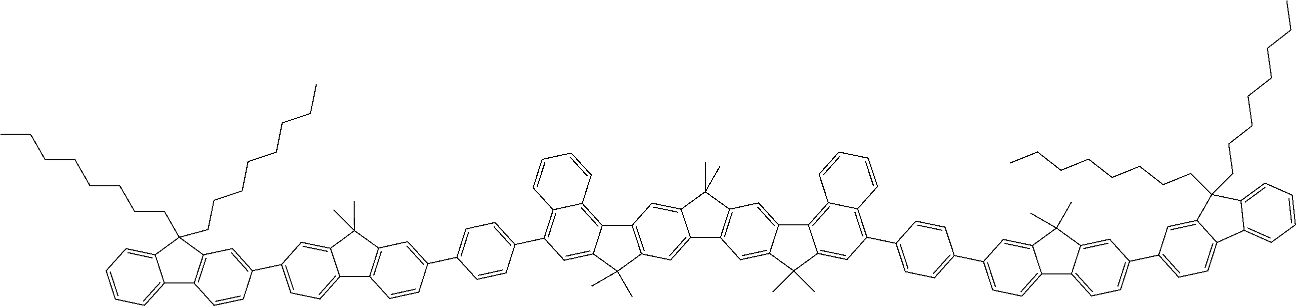 [SMILES: CCCCCCCCC1(CCCCCCCC)c2ccccc2-c2ccc(-c3ccc4c(c3)C(C)(C)c3cc(-c5ccc(-c6cc7c(c8ccccc68)-c6cc8c(cc6C7(C)C)-c6cc7c(cc6C8(C)C)-c6c(cc(-c8ccc(-c9ccc%10c(c9)C(C)(C)c9cc(-c%11ccc%12c(c%11)C(CCCCCCCC)(CCCCCCCC)c%11ccccc%11-%12)ccc9-%10)cc8)c8ccccc68)C7(C)C)cc5)ccc3-4)cc21]

| | |
|---|---|
| 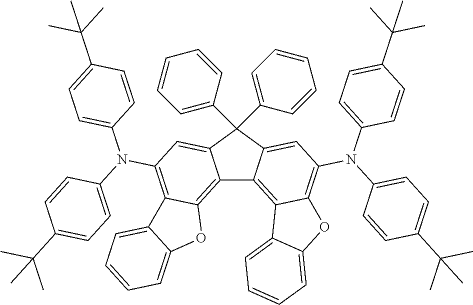 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C3(c2ccccc2)c2ccccc2)cc1] | 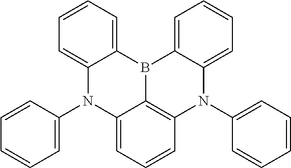 [SMILES: c1ccc(N2c3ccccc3B3c4ccccc4N(c4ccccc4)c4cccc2c43)cc1] |

The compounds according to formula (III) or (IV) can also be employed in other layers, for example as hole-transport materials in a hole-injection or hole-transport layer or electron-blocking layer or as matrix materials in an emitting layer, preferably as matrix materials for phosphorescent emitters.

If the compound of the formula (III) or (IV) is employed as hole-transport material in a hole-transport layer, a hole-injection layer or an electron-blocking layer, the compound can be employed as pure material, i.e. in a proportion of 100%, in the hole-transport layer, or it can be employed in combination with one or more further compounds. According to a preferred embodiment, the organic layer comprising the compound of the formula (III) or (IV) then additionally comprises one or more p-dopants. The p-dopants employed in accordance with the present invention are preferably organic electron-acceptor compounds which are able to oxidise one or more of the other compounds of the mixture.

Particularly preferred embodiments of p-dopants are the compounds disclosed in WO 2011/073149, EP 1968131, EP 2276085, EP 2213662, EP 1722602, EP 2045848, DE 102007031220, U.S. Pat. Nos. 8,044,390, 8,057,712, WO 2009/003455, WO 2010/094378, WO 2011/120709, US 2010/0096600 and WO 2012/095143.

If the compound of the formula (III) or (IV) is employed as matrix material in combination with a phosphorescent emitter in an emitting layer, the phosphorescent emitter is preferably selected from the classes and embodiments of phosphorescent emitters indicated below. Furthermore, one or more further matrix materials are preferably present in the emitting layer in this case.

So-called mixed-matrix systems of this type preferably comprise two or three different matrix materials, particularly preferably two different matrix materials. It is preferred here for one of the two materials to be a material having hole-transporting properties and for the other material to be a material having electron-transporting properties.

However, the desired electron-transporting and hole-transporting properties of the mixed-matrix components may also be combined mainly or completely in a single mixed-matrix component, where the further mixed-matrix component or components satisfy other functions. The two different matrix materials may be present here in a ratio of 1:50 to 1:1, preferably 1:20 to 1:1, particularly preferably 1:10 to 1:1 and very particularly preferably 1:4 to 1:1. Mixed-matrix systems are preferably employed in phosphorescent organic electroluminescent devices. Further details on mixed-matrix systems are contained, inter alia, in the application WO 2010/108579.

Particularly suitable matrix materials which can be used as matrix components of a mixed-matrix system in combination with the compounds according to the invention are selected from the preferred matrix materials for phosphorescent emitters indicated below or the preferred matrix materials for fluorescent emitters, depending on what type of emitter compound is employed in the mixed-matrix system.

Generally preferred classes of material for use as corresponding functional materials in the organic electroluminescent devices according to the invention are indicated below.

Suitable phosphorescent emitters are, in particular, compounds which emit light, preferably in the visible region, on suitable excitation and in addition contain at least one atom having an atomic number greater than 20, preferably greater than 38 and less than 84, particularly preferably greater than 56 and less than 80. The phosphorescent emitters used are preferably compounds which contain copper, molybdenum, tungsten, rhenium, ruthenium, osmium, rhodium, iridium, palladium, platinum, silver, gold or europium, in particular compounds which contain iridium, platinum or copper.

For the purposes of the present invention, all luminescent iridium, platinum or copper complexes are regarded as phosphorescent compounds.

Examples of the phosphorescent emitters are described in the applications WO 2000/70655, WO 2001/41512, WO 2002/02714, WO 2002/15645, EP 1191613, EP 1191612, EP 1191614, WO 2005/033244, WO 2005/019373 and US 2005/0258742. In general, all phosphorescent complexes as used in accordance with the prior art for phosphorescent OLEDs and as are known to the person skilled in the art in the area of organic electroluminescent devices are suitable for use in the devices according to the invention. The person 3_skilled in the art will also be able to employ further phosphorescent complexes without inventive step in combination with the compounds according to the invention in OLEDs.

Preferred matrix materials for phosphorescent emitters are aromatic ketones, aromatic phosphine oxides or aromatic sulfoxides or sulfones, for example in accordance with WO 2004/013080, WO 2004/093207, WO 2006/005627 or WO 2010/006680, triarylamines, carbazole derivatives, for example CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example in accordance with WO 2007/063754 or WO 2008/056746, indenocarbazole derivatives, for example in accordance with WO 2010/136109, WO 2011/000455 or WO 2013/041176, azacarbazole derivatives, for example in accordance with EP 1617710, EP 1617711, EP 1731584, JP 2005/347160, bipolar matrix materials, for example in accordance with WO 2007/137725, silanes, for example in accordance with WO 2005/111172, azaboroles or boronic esters, for example in accordance with WO 2006/117052, triazine derivatives, for example in accordance with WO 2010/015306, WO 2007/063754 or WO 2008/056746, zinc complexes, for example in accordance with EP 652273 or WO 2009/062578, diazasilole or tetraazasilole derivatives, for example in accordance with WO 2010/054729, diazaphosphole derivatives, for example in accordance with WO 2010/054730, bridged carbazole derivatives, for example in accordance with US 2009/0136779, WO 2010/050778, WO 2011/042107, WO 2011/088877 or WO 2012/143080, triphenylene derivatives, for example in accordance with WO 2012/048781, or lactams, for example in accordance with WO 2011/116865 or WO 2011/137951.

Besides the compounds according to the invention, suitable charge-transport materials, as can be used in the hole-injection or hole-transport layer or electron-blocking layer or in the electron-transport layer of the electronic device according to the invention, are, for example, the compounds disclosed in Y. Shirota et al., Chem. Rev. 2007, 107(4), 953-1010, or other materials as are employed in these layers in accordance with the prior art.

Materials which can be used for the electron-transport layer are all materials as are used in accordance with the prior art as electron-transport materials in the electron-transport layer. Particularly suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Furthermore suitable materials are derivatives of the above-mentioned compounds, as disclosed in JP 2000/053957, WO 2003/060956, WO 2004/028217, WO 2004/080975 and WO 2010/072300.

Preferred hole-transport materials which can be used in a hole-transport, hole-injection or electron-blocking layer in the electroluminescent device according to the invention are indenofluorenamine derivatives (for example in accordance with WO 06/122630 or WO 06/100896), the amine derivatives disclosed in EP 1661888, hexaazatriphenylene derivatives (for example in accordance with WO 01/049806), amine derivatives containing condensed aromatic rings (for example in accordance with U.S. Pat. No. 5,061,569), the amine derivatives disclosed in WO 95/09147, monobenzoindenofluorenamines (for example in accordance with WO 08/006449), dibenzoindenofluorenamines (for example in accordance with WO 07/140847), spirobifluorenamines (for example in accordance with WO 2012/034627 or WO 2013/120577), fluorenamines (for example in accordance with the as yet unpublished applications EP 12005369.9, EP 12005370.7 and EP 12005371.5), spirodibenzopyranamines (for example in accordance with WO 2013/083216) and dihydroacridine derivatives (for example in accordance with WO 2012/150001). The compounds according to the invention can also be used as hole-transport materials.

The preferred embodiments with regard to the organic electroluminescent device in terms of cathode, anode, fabrication processes and applications are the same as those described above.

The invention will now be explained in greater detail by the following examples, without wishing to restrict it thereby.

### A) SYNTHESES EXAMPLES

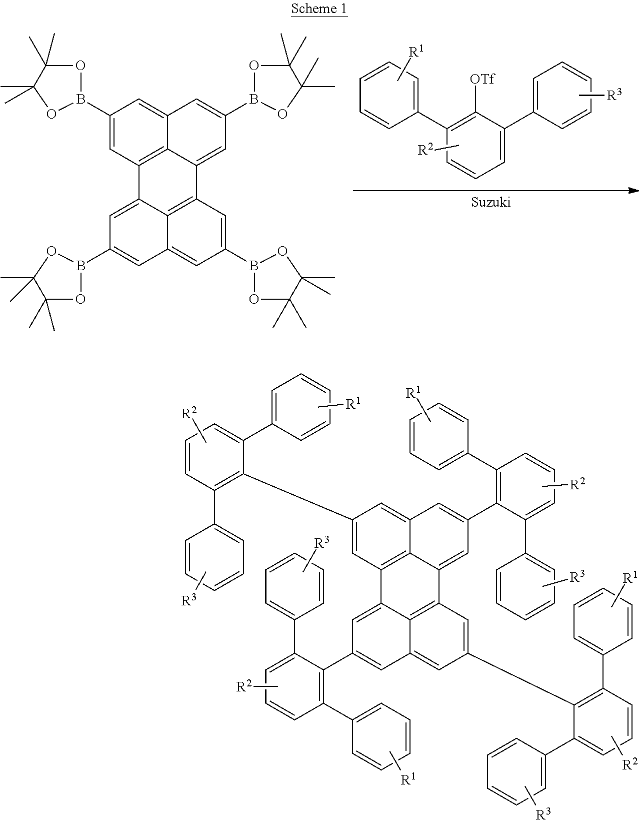

### Synthesis of Triflate Coupling Partner:

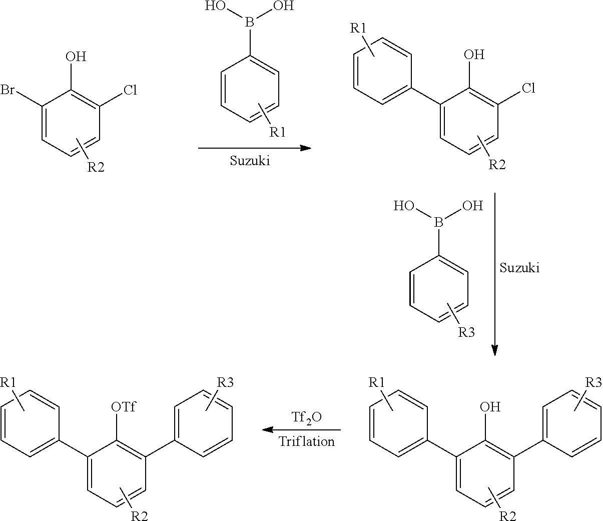

### Example Synthesis of Triflate Coupling Partner

### 3-chloro-4′-methyl-[1,1′-biphenyl]-2-ol

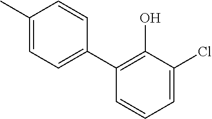

Under an argon atmosphere, an oven dried flask is equipped with 2-bromo-6-chlorophenol (100.0 g, 0.48 mol, 1.0 equiv.), 4-methylphenyl-boronic acid (65.3 g, 0.48 mol, 1.0 equiv.), potassium carbonate (200.0 g, 1.45 mol, 3.0 equiv.) and bis(tri-tert-butylphosphine)palladium(0) (5.1 g, 0.01 mmol, 0.02 equiv). Toluene (1500 mL) and water (500 mL) are added and the reaction mixture is refluxed for 24 h. The organic phase is separated and concentrated. The crude product is purified by column chromatography. The desired product is obtained as a white solid (100.6 g, 0.46, 96%).

### 3′,5′-dimethyl-3-(4-methylphenyl)-[1,1′-biphenyl]-2-ol

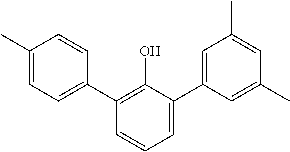

Under an argon atmosphere, an oven dried flask is equipped with 3-chloro-4′-methyl-[1,1′-biphenyl]-2-ol (100.0 g, 0.46 mol, 1.0 equiv.), 3,5-dimethylphenyl-boronic acid (149.98, 67.0 g, 1.0 equiv.), potassium carbonate (193.5 g, 1.38 mmol, 3.0 equiv.) and chloro[(tricyclohexylphosphine)-2-(2′-aminobiphenyl)]palladium(II) (5.9 g, 0.01 mmol, 0.02 equiv). Toluene (1500 mL) and water (500 mL) are added and the reaction mixture is refluxed for 24 h. The organic phase is separated and concentrated. The crude product is purified by column chromatography. The desired product is obtained as a white solid (119.4 g, 0.41 mol, 90%)

### 3′,5′-dimethyl-3-(4-methylphenyl)-[1,1′-biphenyl]-2-yl trifluoromethanesulfonate

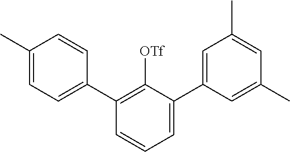

Under an argon atmosphere, an oven dried flask is equipped with 3′,5′-dimethyl-3-(4-methylphenyl)-[1,1′-biphenyl]-2-ol (110 g, 0.38 mol, 1.0 equiv.) in DCM (1000 mL). The mixture is cooled to 0° C. Pyridine (60. g, 61.3 mL, 0.76 mol, 2.0 equiv.) is added. Then trifluoromethanesulfonic anhydride (130.0 g, 77.5 mL, 0.46 mol, 1.2 equiv.) in DCM (300 mL) is added slowly. The reaction mixture is allowed to warm to rt overnight. The reaction mixture is washed with 3 M hydrochloric acid (400 mL) and saturated sodium hydrogen carbonate solution (400 mL). The organic phase is concentrated. The crude product is purified by recrystallization from methanol. The desired product is obtained as white solid (143.0 g, 0.34 mol, 90%).

### 2,5,8,11-Tetrakis(2,6-dimethyl-phenyl)-perylene

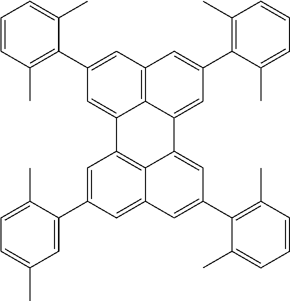 [SMILES: Cc1ccc(C)c(-c2cc3cc(-c4c(C)cccc4C)cc4c5cc(-c6c(C)cccc6C)cc6cc(-c7c(C)cccc7C)cc(c(c2)c34)c65)c1]

Under an argon atmosphere, an oven dried flask is equipped with a magnetic stir bar, 2,5,8,11-tetra-(4,4,5,5-tetramethyl-1,3,2-dioxaborolan-2-yl)-perylene (40.0 g, 52.9 mmol, 1.0 equiv.), 2-Bromo-1,3-dimethyl-benzene (293.7 g, 212.8 mL, 1587.0 mmol, 30.0 equiv.) and cesium carbonate (137.9 g, 423.2 mmol, 8.0 equiv.). Toluene (2000 mL) is then added and the reaction mixture is degassed with Ar. Tetrakis(triphenylphoshine)palladium (6.11 g, 5.3 mmol, 0.1 equiv.) is then added and the reaction mixture is stirred with heating to reflux for 72 h. The resulting precipitate is filtered off, and methanol (1000 ml) is added to the filtrate. The resulting precipitate is collected and the combined precipitates are purified by hot extraction, recrystallization and sublimation. The desired product is thus isolated as a yellow solid (4.5 g, 6.73 mmol, 12.7%)

### 2,5,8,11-Tetrakis(2,6-diphenyl-phenyl)-perylene

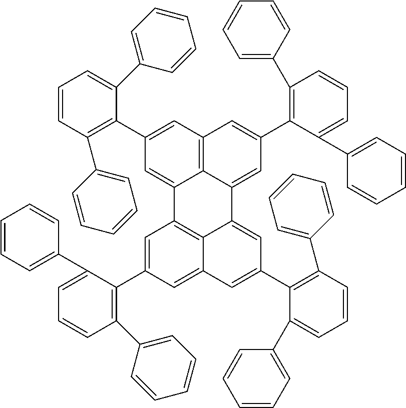 [SMILES: c1ccc(-c2cccc(-c3ccccc3)c2-c2cc3cc(-c4c(-c5ccccc5)cccc4-c4ccccc4)cc4c5cc(-c6c(-c7ccccc7)cccc6-c6ccccc6)cc6cc(-c7c(-c8ccccc8)cccc7-c7ccccc7)cc(c(c2)c34)c65)cc1]

Under an argon atmosphere, an oven dried flask is equipped with a magnetic stir bar, 2,5,8,11-tetra-(4,4,5,5-tetramethyl-1,3,2-dioxaborolan-2-yl)-perylene (38.0 g, 50.3 mmol, 1.0 equiv.), 3-phenyl-[1,1′-biphenyl]-2-yl-trifluoromethanesulfonate (95.1 g, 251.3 mmol, 5.0 equiv.) and sodium metaborate tetrahydrate (69.3 g, 502.5 mmol, 10.0 equiv.). THF (1500 mL) and water (500 mL) are then added and the reaction mixture is degassed with Ar. Tetrakis(triphenylphoshine)palladium (5.81 g, 5.0 mmol, 0.1 equiv.) is then added and the reaction mixture is stirred with heating to reflux for 72 h. The reaction mixture is cooled to RT and the organic phase is collected and concentrated. The crude product is purified by hot extraction, recrystallization and sublimation. The desired product is thus isolated as a yellow solid (6.8 g, 5.8 mmol, 11.6%).

### B) FABRICATION OF OLEDS

Glass plates coated with structured ITO (50 nm, indium tin oxide) are wet-cleaned (dishwasher, Merck Extran cleaner). The substrates are then treated with UV/ozone for 15 minutes. A 20 nm PEDOT:PSS layer is then spin-coated onto the substrates (2800 U/min). The substrates are dried again for 10 minutes on the hot plate at 180° C. After the fabrication, the OLEDs are encapsulated for protection against oxygen and water vapor. The exact layer structure of the OLEDs (organic light emitting diodes) can be found in the examples. The materials used to prepare the OLEDs are shown in Table 2.

All materials are thermally evaporated in a vacuum chamber. In this case, the emission layer(s) always consist(s) of at least one matrix material (host material), a phosphorescent sensitizer (PS) and a fluorescent emitter (FE). Sensitizer and fluorescent emitter (FE) are added to the host material (H) by co-evaporation in a certain volume fraction. An indication such as H-01:PS-01(5%):FE-01(3%) means that the material H-01 is present in a volume fraction of 92%, PS-01 is present in a volume fraction of 5% and FE-01 is present in a volume fraction of 3% in the layer. Similarly, the electron transport layer may consist of a mixture of two materials.

The OLEDs are characterised by standard methods. For this purpose, the electroluminescence spectra are recorded, the current efficiency (measured in cd/A) and the external quantum efficiency (EQE, measured in percent) as a function of the luminous density assuming Lambert emission characteristics are calculated from current/voltage/luminous density characteristic lines (IUL characteristic lines). The indication U100 indicates the voltage required for a luminance of 100 cd/m². EQE100 refers to the external quantum efficiency at an operating luminance of 100 cd/m².

The phosphorescent sensitizers used are the compounds PS-01 and PS-02. The fluorescent emitters used are the compounds FE-01, FE-02 and FE-03.

OLEDs with Blue Emission:

OLEDs consist of the following layer sequence, which is applied to the substrate after the PEDOT:PSS-treatment:

20 nm HTM:pD (95%:5%), 30 nm HTM, 10 nm H-02, 25 nm H-01:PS:FE, 10 nm H-01, 20 nm ETM:LiQ (50%:50%), aluminum (100 nm).

Table 1 below lists the results for various combinations of host, sensitizer and fluorescent emitter. The EQE and voltage at 100 cd/in² are given for the respective experiments.

TABLE 1
Experiments with blue emitting OLEDs

| | | | | EQE100 | U100 |
|---|---|---|---|---|---|
| Exp. | Host | Sensitizer | FE | [%] | [V] |
| 1 | H-01 | PS-01 (15%) | FE-01 | 10.16 | 3.78 |
| | | | (1%) | | |
| 2 | H-01 | PS-01 (15%) | FE-01 | 6.77 | 3.91 |
| | | | (2%) | | |
| 3 | H-01 | PS-01 (15%) | FE-01 | 5.34 | 4 |
| | | | (3%) | | |
| 4 | H-01 | PS-01 (15%) | FE-02 | 9.32 | 3.89 |
| | | | (2%) | | |
| 5 | H-01 | PS-01 (15%) | FE-02 | 7.7 | 4.06 |
| | | | (3%) | | |
| 6 | H-01 | PS-01 (15%) | FE-03 | 19.67 | 3.63 |
| | | | (1%) | | |
| 7 | H-01 | PS-01 (15%) | FE-03 | 17.93 | 3.67 |
| | | | (2%) | | |
| 8 | H-01 | PS-01 (15%) | FE-03 | 14.77 | 3.68 |
| | | | (3%) | | |
| 9 | H-01 | PS-02 (5%) | FE-01 | 13 | 3.33 |
| | | | (1%) | | |
| 10 | H-01 | PS-02 (5%) | FE-01 | 9.5 | 3.37 |
| | | | (2%) | | |
| 11 | H-01 | PS-02 (5%) | FE-01 | 8.4 | 3.4 |
| | | | (3%) | | |
| 12 | H-01 | PS-02 (5%) | FE-02 | 12.3 | 3.28 |
| | | | (2%) | | |
| 13 | H-01 | PS-02 (5%) | FE-02 | 11.2 | 3.28 |
| | | | (3%) | | |
| 14 | H-01 | PS-02 (5%) | FE-03 | 18.7 | 3.23 |
| | | | (1%) | | |
| 15 | H-01 | PS-02 (5%) | FE-03 | 15.8 | 3.24 |
| | | | (2%) | | |
| 16 | H-01 | PS-02 (5%) | FE-03 | 13.5 | 3.28 |
| | | | (3%) | | |

### Results

Table 1 shows that blue-emitting OLEDs comprising FE-TM, FE-02 and FE-03 as fluorescent emitters in an emission layer containing a phosphorescent sensitizer are performant in terms of efficiency (EQE) and operating voltage (U100). More particularly, blue emitting OLEDs comprising FE-02 and FE-03, especially FE-03, achieve excellent results in terms of efficiency, while the operating voltage is relatively low.

TABLE 2
Structures of the OLED materials

| | |
|---|---|
| 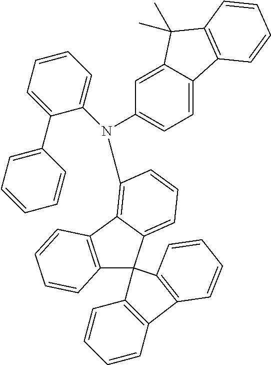 [SMILES: CC1(C)c2ccccc2-c2ccc(N(c3ccccc3-c3ccccc3)c3cccc4c3-c3ccccc3C43c4ccccc4-c4ccccc43)cc21] | HTM |
| 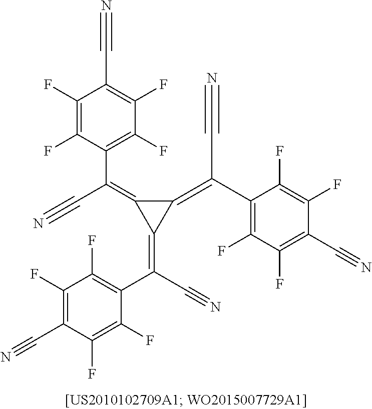 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] | p-D |
| 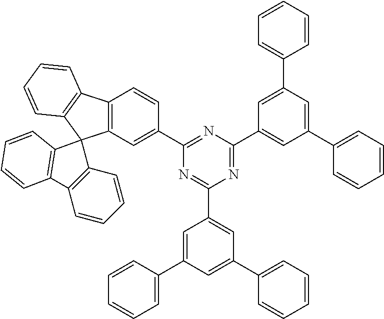 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | ETM |
| 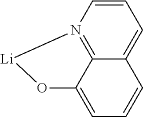 | LiQ |
| 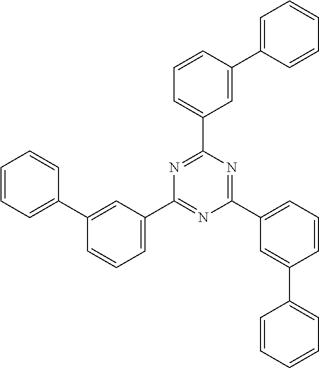 [SMILES: c1ccc(-c2cccc(-c3nc(-c4cccc(-c5ccccc5)c4)nc(-c4cccc(-c5ccccc5)c4)n3)c2)cc1] | H-01 |
| 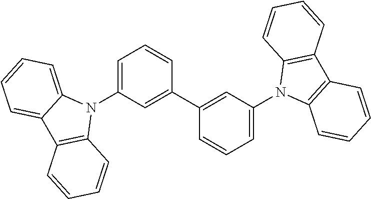 [SMILES: c1cc(-c2cccc(-n3c4ccccc4c4ccccc43)c2)cc(-n2c3ccccc3c3ccccc32)c1] | H-02 |
| 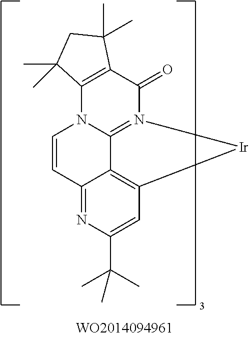 | PS-01 |
| 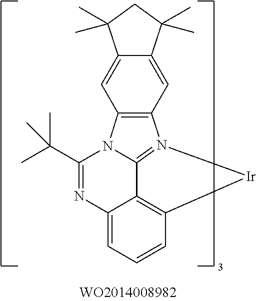 | PS-02 |
| 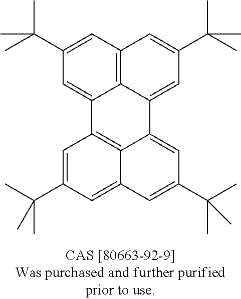 [SMILES: CC(C)(C)c1cc2cc(C(C)(C)C)cc3c4cc(C(C)(C)C)cc5cc(C(C)(C)C)cc(c(c1)c23)c54] | FE-01 |
| 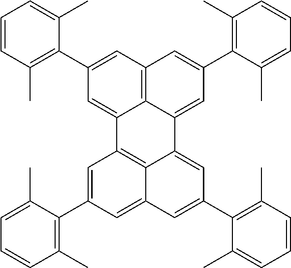 [SMILES: Cc1cccc(C)c1-c1cc2cc(-c3c(C)cccc3C)cc3c4cc(-c5c(C)cccc5C)cc5cc(-c6c(C)cccc6C)cc(c(c1)c23)c54] | FE-02 |
| 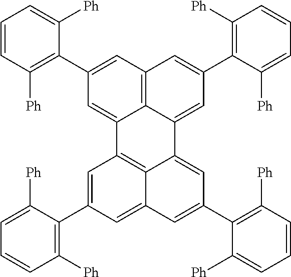 [SMILES: c1ccc(-c2cccc(-c3ccccc3)c2-c2cc3cc(-c4c(-c5ccccc5)cccc4-c4ccccc4)cc4c5cc(-c6c(-c7ccccc7)cccc6-c6ccccc6)cc6cc(-c7c(-c8ccccc8)cccc7-c7ccccc7)cc(c(c2)c34)c65)cc1] | FE-03 |

1. Electronic device comprising anode, cathode and at least one organic layer comprising a sterically hindered fluorescent perylene emitter compound, characterised in that the fluorescent perylene emitter compound is represented by the general following formula (I) and in that the organic layer or a layer adjacent to the organic layer on the anode or cathode side comprises a sensitizer compound selected from a compound that exhibits delayed fluorescence or a phosphorescent compound, 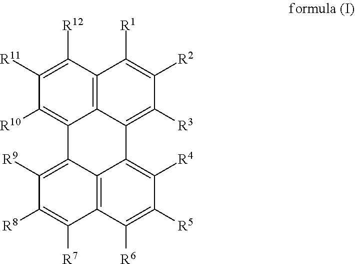
wherein
R¹ to R¹² are each selected, identically or differently, from H, a straight-chain alkyl or alkoxy group having 3 to 20 carbon atoms, a branched or cyclic alkyl or alkoxy group having 3 to 20 carbon atoms, an alkenyl or alkynyl group having 3 to 20 carbon atoms, an aralkyl group, preferably having 7 to 60 carbon atoms, where the above-mentioned groups may each be substituted by one or more radicals R²⁰ and where one or more CH₂ groups in the above-mentioned groups may be replaced by Si(R²⁰)₂, Ge(R²⁰)₂, Sn(R²⁰)₂, C═O, C═S, C═Se, C═NR²⁰, P(═O)(R²⁰), SO, SO₂, NR²⁰, —O—, —S—, —COO— or —CONR²⁰— and where one or more H atoms in the above-mentioned groups may be replaced by D, F, Cl, Br, I, CN or NO₂, or an aromatic ring system having 5 to 60 aromatic ring atoms, which may in each case be substituted by one or more radicals R²⁰;
R²⁰ is on each occurrence, identically or differently, selected from H, D, F, or a straight-chain alkyl group having 1 to 40 carbon atoms, or a branched or cyclic alkyl group having 3 to 40 carbon atoms, or an alkenyl or alkynyl group having 2 to 40 carbon atoms, or an aralkyl group having 7 to 40 carbon atoms, where the above-mentioned groups may each be substituted by one or more radicals R²¹ or an aromatic ring system having 5 to 40 aromatic ring atoms, which may in each case be substituted by one or more radicals R²¹, where two or more radicals R²⁰ may be joined to form an aromatic ring system or a (poly)cyclic alkyl group, which may in each case be substituted by one or more radicals R²¹;
R²¹ is on each occurrence, identically or differently, selected from H, D, F, or a straight-chain alkyl group having 1 to 20 carbon atoms, or a branched or cyclic alkyl group having 3 to 20 carbon atoms, or an alkenyl or alkynyl group having 2 to 20 carbon atoms, or an aromatic ring system having 5 to 30 aromatic ring atoms, where two or more radicals R²¹ may be joined to form an aromatic ring system or a (poly)cyclic alkyl group;
with the proviso that at least two, preferably three, more preferably four, of radicals R¹ to R¹², which are not located at the same benzene ring of the perylene basic skeleton, are other than H.

2. Electronic device according to claim 1, characterized in that the compound of formula (I) represents a compound of general formula (II) 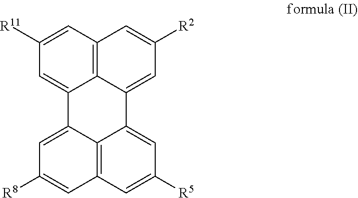
wherein
R², R⁵, R⁸, R¹¹ are each selected, identically or differently, from a straight-chain alkyl or alkoxy group having 3 to 20 carbon atoms, a branched or cyclic alkyl or alkoxy group having 3 to 20 carbon atoms, an alkenyl or alkynyl group having 3 to 20 carbon atoms, an aralkyl group, preferably having 7 to 60 carbon atoms, where the above-mentioned groups may each be substituted by one or more radicals R²⁰ and where one or more CH₂ groups in the above-mentioned groups may be replaced by Si(R²⁰)₂, Ge(R²⁰)₂, Sn(R²⁰)₂, C═O, C═S, C═Se, C═NR²⁰, P(═O)(R²⁰), SO, SO₂, NR²⁰, —O—, —S—, —COO— or —CONR²⁰— and where one or more H atoms in the above-mentioned groups may be replaced by D, F, Cl, Br, I, CN or NO₂, or an aromatic ring system having 5 to 60 aromatic ring atoms, which may in each case be substituted by one or more radicals R²⁰;
R²⁰ is on each occurrence, identically or differently, selected from H, D, F, or a straight-chain alkyl group having 1 to 40 carbon atoms, or a branched or cyclic alkyl group having 3 to 40 carbon atoms, or an alkenyl or alkynyl group having 2 to 40 carbon atoms, or an aralkyl group having 7 to 40 carbon atoms, where the above-mentioned groups may each be substituted by one or more radicals R²¹, or an aromatic ring system having 5 to 40 aromatic ring atoms, which may in each case be substituted by one or more radicals R²¹, where two or more radicals R²⁰ may be joined to form an aromatic ring system or a (poly)cyclic alkyl group, which may in each case be substituted by one or more radicals R²¹;
R²¹ is on each occurrence, identically or differently, selected from H, D, F, or a straight-chain alkyl group having 1 to 20 carbon atoms, or a branched or cyclic alkyl group having 3 to 20 carbon atoms, or an alkenyl or alkynyl group having 2 to 20 carbon atoms, or an aromatic ring system having 5 to 40 aromatic ring atoms, where two or more radicals R²¹ may be joined to form an aromatic ring system or a (poly)cyclic alkyl group.

3. Electronic device according to claim 1 or 2, characterized in that R², R⁵, R⁸, R¹¹ are each selected, identically or differently, from a straight-chain, branched or cyclic alkyl group having 4 to 10 carbon atoms, a straight-chain, branched or cyclic alkoxy group having 3 to 10 carbon atoms, an aralkyl group having 7 to 30 carbon atoms, where the above-mentioned groups may each be substituted by one or more radicals R²⁰ and where one or more H atoms in the above-mentioned groups may be replaced by D, F, Cl or CN, or an aromatic ring system having 6 to 30 aromatic ring atoms, which may in each case be substituted by one or more radicals R²⁰;
R²⁰ is on each occurrence, identically or differently, selected from D, F, or a straight-chain alkyl group having 1 to 20 carbon atoms or a branched or cyclic alkyl group having 3 to 20 carbon atoms or an alkenyl or alkynyl group having 2 to 20 carbon atoms, where the above-mentioned groups may each be substituted by one or more radicals R²¹, or an aromatic ring system having 5 to 30 aromatic ring atoms, which may in each case be substituted by one or more radicals R²¹, where two or more radicals R²⁰ may be joined to form an aromatic ring system or a (poly)cyclic alkyl group, which may in each case be substituted by one or more radicals R²¹;
R²¹ is on each occurrence, identically or differently, selected from H, D, F, or a straight-chain alkyl group having 1 to 10 carbon atoms, or a branched or cyclic alkyl group having 3 to 10 carbon atoms, or an alkenyl or alkynyl group having 2 to 10 carbon atoms, or an aromatic ring system having 5 to 30 aromatic ring atoms, where two or more radicals R²¹ may be joined to form an aromatic ring system or a (poly)cyclic alkyl group.

4. Electronic device according to any one of claims 1 to 3, characterized in that R², R⁵, R⁸, R¹¹ are each selected, identically or differently,
from branched or cyclic alkyl groups represented by the general following formula (R-a) 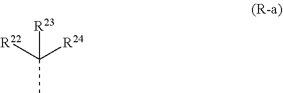
wherein
R²², R²³, R²⁴ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R²⁵, and where two of radicals R²², R²³, R²⁴ or all radicals R²², R²³, R²⁴ may be joined to form a (poly)cyclic alkyl group, which may be substituted by one or more radicals R²⁵;
R²⁵ is at each occurrence, identically or differently, selected from a straight-chain alkyl group having 1 to 10 carbon atoms, or a branched or cyclic alkyl group having 3 to 10 carbon atoms;
with the proviso that at each occurrence at least one of radicals R²², R²³ and R²⁴ is other than H, with the proviso that at each occurrence all of radicals R²², R²³ and R²⁴ together have at least 4 carbon atoms and with the proviso that at each occurrence, if two of radicals R²², R²³, R²⁴ are H, the remaining radical is not a straight-chain;
or from branched or cyclic alkoxy groups represented by the general following formula (R-b) 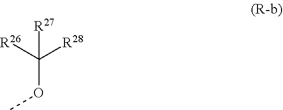
wherein
R²⁶, R²⁷, R²⁸ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R²⁵ as defined above, and where two of radicals R²⁶, R²⁷, R²⁸ or all radicals R²⁶, R²⁷, R²⁸ may be joined to form a (poly)cyclic alkyl group, which may be substituted by one or more radicals R²⁵ as defined above;
with the proviso that at each occurrence only one of radicals R²⁶, R²⁷ and R²⁸ may be H;
or from aralkyl groups represented by the general following formula (R-c) 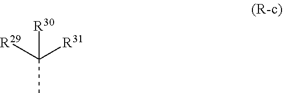
wherein
R²⁹, R³⁰, R³¹ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 30 aromatic ring atoms, which may in each case be substituted by one or more radicals R³², and where two or all of radicals R²⁹, R³⁰, R³¹ may be joined to form a (poly)cyclic alkyl group or an aromatic ring system, each of which may be substituted by one or more radicals R³²;
R³² is at each occurrence, identically or differently, selected from a straight-chain alkyl group having 1 to 10 carbon atoms, or a branched or cyclic alkyl group having 3 to 10 carbon atoms, or an aromatic ring system having 6 to 24 aromatic ring atoms;
with the proviso that at each occurrence at least one of radicals R²⁹, R³⁰ and R³¹ is other than H and that at each occurrence at least one of radicals R²⁹, R³⁰ and R³¹ is or contains an aromatic ring system having at least 6 aromatic ring atoms;
or from aromatic ring systems represented by the general following formula (R-d) 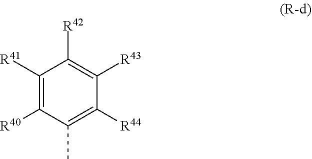
wherein
R⁴⁰ to R⁴⁴ is at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 30 aromatic ring atoms, which may in each case be substituted by one or more radicals R³², and where two or more of radicals R⁴⁰ to R⁴⁴ may be joined to form a (poly)cyclic alkyl group or an aromatic ring system, each of which may be substituted by one or more radicals R³² as defined above.

5. Electronic device according to any one of claims 1 to 4, characterized in that R², R⁵, R⁸, R¹¹ are identical.

6. Electronic device according to any one of claims 1 to 5, characterized in that the compound of formula (I) represents a compound of general formulae (III) or (IV) 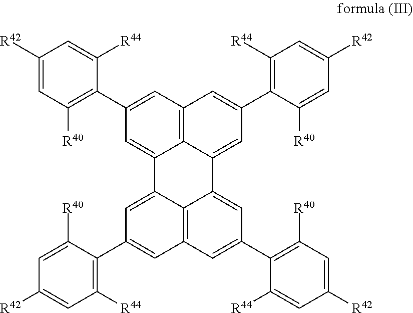
wherein
R⁴⁰, R⁴², R⁴⁴ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 30 aromatic ring atoms, which may in each case be substituted by one or more radicals R³²; where R³² is as defined in claim 4;
with the proviso that at least one of R⁴⁰, R⁴², R⁴⁴ is other than H;
or 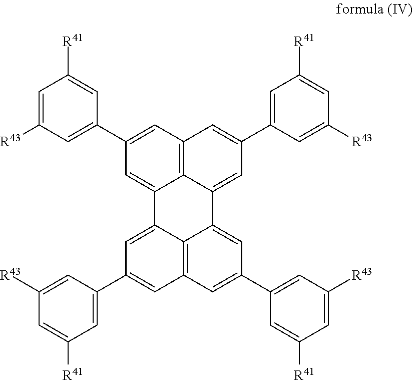
wherein
R⁴¹, R⁴³ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 30 aromatic ring atoms, which may in each case be substituted by one or more radicals R³²; where R³² is as defined in claim 4;
with the proviso that at least one of R⁴¹, R⁴³ is other than H.

7. Electronic device according to any one of claims 1 to 6, characterized in that
R⁴² is at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³²;
R⁴⁰, R⁴⁴ are at each occurrence, identically or differently, selected from an aromatic ring system having 6 to 30 aromatic ring atoms, which may in each case be substituted by one or more radicals R³²; where R³² is as defined in claim 4.

8. Electronic device according to any one of claims 1 to 6, characterized in that
R⁴⁰, R⁴², R⁴⁴ are at each occurrence, identically or differently, selected from an aromatic ring system having 6 to 30 aromatic ring atoms, which may in each case be substituted by one or more radicals R³²; where R³² is as defined in claim 4.

9. Electronic device according to any one of claims 1 to 8, characterized in that the compound of formula (I) represents any one of a compound of general formulae (IIIa), (IIIb) or (IIIc) 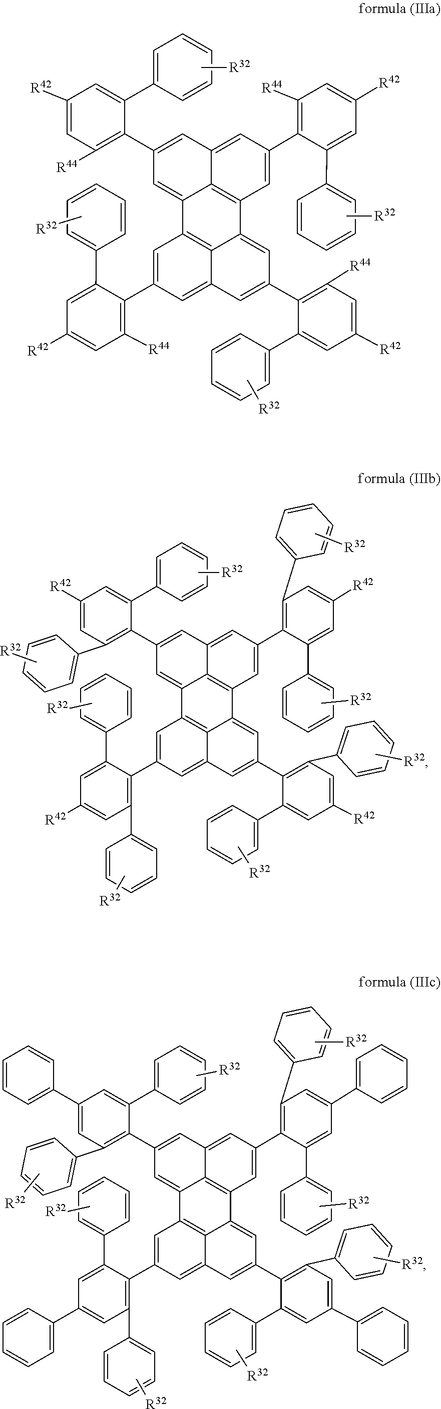
wherein
in each of formulae (IIIa), (IIIb) and (IIIc) the phenyl groups indicated with —R³² are unsubstituted or substituted with one or more radicals R³²;
R⁴² and R⁴⁴ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³²; where R³² is as defined in claim 4.

10. Electronic device according to any one of claims 1 to 6, characterized in that
R⁴² is at each occurrence, identically or differently, selected from H, a straight-chain alkyl group having 1 to 10 carbon atoms, or a branched or cyclic alkyl group having 3 to 10 carbon atoms, or an aromatic ring system having 6 to 30 aromatic ring atoms, which may in each case be substituted by one or more radicals R³²;
R⁴⁰, R⁴⁴ are at each occurrence identically selected from a straight-chain alkyl group having 1 to 10 carbon atoms, or a branched or cyclic alkyl group having 3 to 10 carbon atoms, which may in each case be substituted by one or more radicals R³²; and R³² is as defined in claim 4.

11. Electronic device according to any one of claims 1 to 10, characterized in that the organic layer comprises the sterically hindered fluorescent emitter compound and the sensitizer compound, said organic layer preferably being the emitting layer.

12. Electronic device according to any one of claims 1 to 11, characterized in that the organic layer comprises the sterically hindered fluorescent emitter compound, the sensitizer compound and at least one organic functional material selected from the group consisting of HTM, HIM, HBM, p-dopant, ETM, EIM, EBM, n-dopant, fluorescent emitter, phosphorescent emitter, delayed fluorescent material, matrix material, host material, wide band gap material, quantum material (preferably quantum dot), said organic layer preferably being the emitting layer.

13. Compound of the formula (III) or (IV) as defined in claim 6, characterised in that the radicals R⁴⁰, R⁴², R⁴⁴ and R⁴¹, R⁴³ are defined as follows: R⁴⁰ to R⁴⁴ are at each occurrence, identically or differently, selected from H, a straight-chain alkyl group having 1 to 10 carbon atoms, or a branched or cyclic alkyl group having 3 to 10 carbon atoms, where the above-mentioned groups may each be substituted by one or more radicals R³², or an aromatic ring system having 6 to 24 aromatic ring atoms, which may in each case be substituted by one or more radicals R³²; and
R³² is at each occurrence, identically or differently, selected from a straight-chain alkyl group having 1 to 10 carbon atoms, or a branched or cyclic alkyl group having 3 to 10 carbon atoms, or an aromatic ring system having 6 to 24 aromatic ring atoms.

14. Compound according to claim 13, characterised in that the radicals R⁴⁰, R⁴², R⁴⁴ are defined as follows:
R⁴² is at each occurrence, identically or differently, selected from H, a straight-chain alkyl group having 1 to 10 carbon atoms, or a branched alkyl group having 3 to 10 carbon atoms;
R⁴⁰, R⁴⁴ are at each occurrence, identically or differently, selected from an aromatic ring system having 6 to 24 aromatic ring atoms, which may in each case be substituted by one or more radicals R³²;
R³² is at each occurrence, identically or differently, selected from a straight-chain alkyl group having 1 to 6 carbon atoms, or a branched alkyl group having 3 to 6 carbon atoms.

15. Compound according to claim 13, characterised in that the radicals R⁴⁰, R⁴², R⁴⁴ are defined as follows:
R⁴⁰, R⁴², R⁴⁴ are at each occurrence, identically or differently, selected from an aromatic ring system having 6 to 24 aromatic ring atoms, which may in each case be substituted by one or more radicals R³²;
R³² is at each occurrence, identically or differently, selected from a straight-chain alkyl group having 1 to 6 carbon atoms, or a branched alkyl group having 3 to 6 carbon atoms.

16. Compound according to any one of claims 13 to 15, characterized in that the compound of formula (III) represents any one of a compound of general formulae (IIId), (IIIe) or (IIIf) 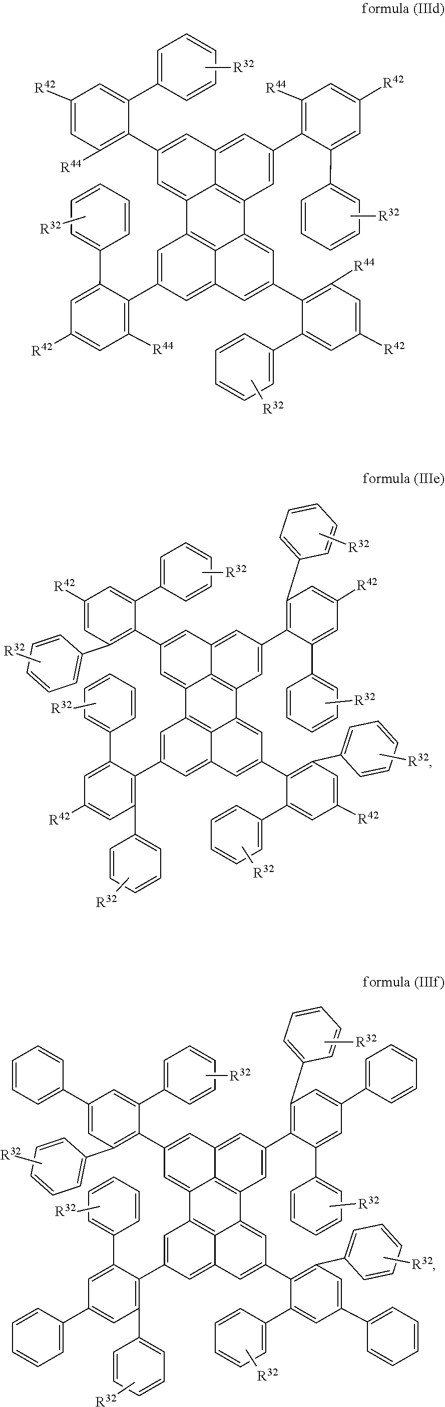
in each of formulae (IIId), (IIIe) and (IIIf) the phenyl groups indicated with —R³² are unsubstituted or substituted with one or more radicals R³²;
R⁴² and R⁴⁴ are at each occurrence, identically or differently, selected from H, a straight-chain alkyl group having 1 to 10 carbon atoms, or a branched alkyl group having 3 to 10 carbon atoms, where the above-mentioned groups may each be substituted by one or more radicals R³²; and
R³² is at each occurrence, identically or differently, selected from a straight-chain alkyl group having 1 to 6 carbon atoms, or a branched alkyl group having 3 to 6 carbon atoms.

17. Compound according to claim 13, characterised in that the radicals R⁴⁰, R⁴², R⁴⁴ are defined as follows:
R⁴² is at each occurrence, identically or differently, selected from H, a straight-chain alkyl group having 1 to 10 carbon atoms, or a branched alkyl group having 3 to 10 carbon atoms, or an aromatic ring system having 6 to 24 aromatic ring atoms, which may in each case be substituted by one or more radicals R³²;
R⁴⁰, R⁴⁴ are at each occurrence, identically or differently, selected from a straight-chain alkyl group having 1 to 10 carbon atoms, or a branched alkyl group having 3 to 10 carbon atoms, which may in each case be substituted by one or more radicals R³²; and
R³² is at each occurrence, identically or differently, selected from a straight-chain alkyl group having 1 to 6 carbon atoms, or a branched alkyl group having 3 to 6 carbon atoms.

18. Compound according to claim 17, characterised in that the radicals R⁴⁰, R⁴², R⁴⁴ are defined as follows:
R⁴² is at each occurrence identically selected from H, a straight-chain alkyl group having 1 to 10 carbon atoms, or a branched alkyl group having 3 to 10 carbon atoms,
R⁴⁰, R⁴⁴ are at each occurrence identically selected from a straight-chain alkyl group having 1 to 10 carbon atoms, or a branched alkyl group having 3 to 10 carbon atoms.

19. Composition comprising a compound according to one or more of claims 13 to 18 and at least one organic or inorganic functional material selected from the group consisting of HTM, HIM, HBM, p-dopant, ETM, EIM, EBM, n-dopant, fluorescent emitter, phosphorescent emitter, delayed fluorescent material, matrix material, host material, wide band gap material, quantum material (preferably quantum dot).

20. Formulation comprising at least one compound according to any one of claims 13 to 18 or a composition according to claim 19 and at least one solvent.

21. Process for the preparation of the compounds of the formula (III) according to any one of claims 13 to 18, characterised in that at least the following step a) is carried out:
a) Organometallic coupling under Suzuki conditions between the 1-C, 5-C, 8-C and 11-C atoms of the perylene basic skeleton and a substituted or unsubstituted aromatic group Ar having 6 to 24 aromatic ring atoms, which is employed as starting material Ar—X, where X is any desired suitable leaving group, preferably selected from a halide, a boronic acid, a boronic ester, a tosylate or a triflate.

22. Use of a compound according to any one of claims 13 to 18, or a composition according to claim 19, or a formulation according to claim 20 in an electronic device, preferably in an organic electroluminescent device.

23. Electronic device comprising a composition according to claim 19, or a formulation according to claim 20, or a compound according to any one of claims 13 to 18.

24. Electronic device according to any of claims 1 to 12 or 23, which is preferably an organic electroluminescent device selected from organic integrated circuits (OICs), organic field-effect transistors (OFETs), organic thin-film transistors (OTFTs), organic light-emitting transistors (OLETs), organic solar cells (OSCs), organic optical detectors, organic photo-receptors, organic field-quench devices (OFQDs), organic light-emitting electrochemical cells (OLECs, LECs, LEECs), organic laser diodes (O-lasers) and organic light emitting diodes (OLEDs).
